(19) United States
(12) Patent Application Publication
(10) Pub. No.: US 2019/0262453 A1
(43) Pub. Date: Aug. 29, 2019

(54) DESIGN AND COMPOSITION OF CELL-STABILIZED PHARMACEUTICAL FORMULATIONS

(71) Applicants: THE REGENTS OF THE UNIVERSITY OF MICHIGAN, ANN ARBOR, MI (US); THE BOARD OF TRUSTEES OF THE UNIVERSITY OF ILLINOIS, URBANA, IL (US)

(72) Inventors: Gustavo Rosania, Ann Arbor, MI (US); Paul J.A. Kenis, Champaign, IL (US); Tehetina Woldemichael, Ann Arbor, MI (US); Mikhail M. Murashov, Howell, MI (US); Phillip Rzeczycki, Ann Arbor, MI (US); Rahul K. Keswani, Canton, MI (US); Elizabeth M. Horstman, Champaign, IL (US)

(21) Appl. No.: 16/344,262

(22) PCT Filed: Oct. 24, 2017
(86) PCT No.: PCT/US2017/058017

§ 371 Date: Apr. 23, 2019

Related U.S. Application Data
(60) Provisional application No. 62/412,461, filed on Oct. 25, 2016.

(51) Int. Cl. A61K 47/02; A61K 9/107; A61K 31/498; A61K 9/127; A61P 29/00

(52) U.S. Cl. CPC A61K 47/02; A61K 9/107; A61P 29/00; A61K 9/127; A61K 31/498

(57) ABSTRACT

Provided herein are physiologically insoluble insoluble forms of drugs. In particular, provided herein are physiologically insoluble insoluble salts of drugs (e.g., basic drugs) and their use in treatment of disease.

### CROSS-REFERENCE TO RELATED APPLICATIONS

The present Application claims priority to U.S. Provisional Patent Application Ser. No. 62/412,461, filed Oct. 25, 2016, the disclosure of which is herein incorporated by reference in its entirety

### STATEMENT REGARDING FEDERALLY SPONSORED RESEARCH OR DEVELOPMENT

This invention was made with government support under GM078200 awarded by the National Institutes of Health. The government has certain rights in the invention.

### FIELD OF THE DISCLOSURE

Provided herein are physiologically insoluble forms of drugs. In particular, provided herein are physiologically insoluble salts of drugs (e.g., basic drugs) and their use in treatment of disease.

### BACKGROUND

Inflammation is associated with many autoimmune, fibrotic, and other diseases (e.g. including but not limited to, artherosclerosis, arthritis, cirrhosis, gout, chronic obstructive pulmonary disease, cancer, Crohn's disease, Inflammatory Bowel Disease, Alzheimer's disease, etc.)

The lung is vulnerable to many inflammatory disorders as it is the only internal organ that is exposed constantly to the external environment. Consequently, respiratory diseases cause an immense worldwide health burden. According to the World Health Organization (WHO) and Forum of International Respiratory Societies (FIRS), it is estimated that ˜15% of the world's population suffers from chronic respiratory conditions, including 235 million people suffering from asthma, more than 200 million from chronic obstructive pulmonary disease (COPD) and these numbers are growing every year. In the case of acute respiratory distress syndrome (ARDS), 200,000 Americans per year are affected, with a mortality rate of 40% but no available therapeutic drug available. Healthcare costs for respiratory diseases are an increasing burden on the economies of all countries, but respiratory diseases are rarely on the public health agenda.

Respiratory diseases, such as ARDS, COPD and asthma, are caused by an uncontrolled inflammatory response characterized by dysregulated pro- and anti-inflammatory mediators and increased numbers and/or altered activation of immune cells, including macrophages. Key inflammatory mediators are the pro-inflammatory cytokines tumor necrosis factor α (TNFα) and interleukin-1 (IL-1α and IL-1β), which are required for the initiation and activation of the immune response, and the anti-inflammatory cytokine interleukin-1 receptor antagonist (IL-1RA), which counteracts IL-1 by competitively binding to IL-1 receptor to block signal transduction and resolve inflammation. Continued dysregulation of TNFα and IL-1 expression is a hallmark of chronic inflammatory disorders of the lung. Macrophages play a critical role in the initiation and resolution of lung inflammation, and importantly, one of the major producers of TNFa and IL-1RA.

Existing anti-inflammatory drugs that act by blocking TNFα activity (e.g., etanercept) or by blocking IL-1 via IL-1RA (e.g., anakinra) are currently undergoing preclinical studies or clinical trials as single agents for the potential treatments for COPD, idiopathic pulmonary fibrosis, asthma, and acute respiratory distress syndrome amongst others. However, both TNFα inhibitors and IL-1RA are soluble agents that when systemically injected are poised to affect the whole body indiscriminately and can lead to serious side effects, including increased susceptibility to infection and sepsis.

Accordingly, new treatments for inflammatory diseases are needed.

### SUMMARY

Provided herein are physiologically insoluble forms of drugs. In particular, provided herein are insoluble (e.g., physiologically insoluble) salts of drugs (e.g., basic drugs) and their use in treatment of disease.

Provided herein are salts (e.g., hydrochloride salts) of weakly basic molecules (e.g., drugs) that are stabilized by physiological, concentrative HCl transport mechanisms. In some embodiments, the salts are stabilized as physiologically insoluble, protonated, membrane-impermeant, and/or aggregated and have a pH max higher than the pH of the surrounding microenvironment. In some embodiments, the salts are stabilized in lysosomes. In some embodiments, the salts are stabilized in macrophage lysosomes. The compositions described herein provide the advantages of decreased solubility, leading to increased in vivo stability and decreased toxicity, and thus find use in the treatment of a variety of disease and conditions.

For example, in some embodiments, the present disclosure provides a composition comprising a compound comprising an insoluble (e.g., physiologically insoluble) hydrochloride salt of a weakly basic molecule. In some embodiments, the compound has a form selected from, for example, an amorphous aggregate, a cell-derived inclusion, a solid particulate, or a crystal. In some embodiments, the compound is stable in a lysosome. In some embodiments, the compound has a pH max higher than the pH of a lysosome. In some embodiments, the compound is membrane-impermeant. In some embodiments, the compound further comprises an organic or inorganic coformer, counterion, solvent molecule or excipient molecule.

In some non-limiting embodiments, the composition is a crystal of a basic pharmaceutical agent and a chloride containing compound, wherein the crystal has a 1:2 ratio of drug molecules to chloride ions or a crystal of a basic pharmaceutical agent, methanol (MeOH), and a chloride containing compound, wherein the crystal has a 1:1:2 ratio of drug molecules to chloride ions to MeOH.

In some embodiments, the crystal has an orthorhombic or monoclinic or triclinic or hexagonal or other crystal structure unit cell. In some embodiments, the crystal has a needle, cube, blade, prism, or rhomboid habit. In some embodiments, the pharmaceutical agent is clofazimine. In some embodiments, the density of the crystals is between 1.15-1.5 g/ml. In some embodiments, the composition further comprises a lipid (e.g., phosphatidylcholine, cholesterol, phosphatidylethanolamine, phosphatidylglycerol, phosphatidylinositol, phosphatidylserine, sphingomyelin, cardiolipin, dioleoylphosphatidylglycerol (DOPG), diacylphosphatidylcholine, diacylphosphatidylethanolamine, ceramide, sphingomyelin, cephalin, cholesterol, cerebrosides, diacylglycerols, dioleoylphosphatidylcholine (DOPC), dimyristoylphosphatidylcholine (DMPC), and dioleoylphosphatidylserine (DOPS), diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palmitoyloleoyl-phosphatidylethanolamine (POPE) palmitoyloleoylphosphatidylcholine (POPC), egg phosphatidylcholine (EPC), di stearoylphosphatidylcholine (DSPC), dipalmitoylphosphatidylcholine (DPPC), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, dioctadecyldimethyl ammonium bromide, polyethylene glycol (PEG), or PEG modified lipids). In some embodiments, the pharmaceutical agent is encapsulated by a liposome comprising the lipid. In some embodiments, the composition further comprises one or more of a non-ionic surfactant, a niosome, a polymer, a protein, or a carbohydrate. In some embodiments, the lipid is modified to comprise a targeting agent selected from, for example, antibodies, mannose, folate, or transferrin.

Further embodiments provide a method of treating or preventing a disease in a subject, comprising: administering any of the aforementioned compositions to a subject diagnosed with or suspected of having a disease. In some embodiments, the administering reduces or eliminates signs and/or or symptoms of the disease. In some embodiments, the disease is cancer (e.g., tumors, blood cancers, etc.), asthma, bronchiolitis, bronchiolitis obliterans, chronic obstructive pulmonary disease (COPD), bronchitis, emphysema, hypersensitivity pneumonitis, idiopathic pulmonary fibrosis, pneumoconiosis, silicosis, meningitis, sepsis, malaria, rheumatoid osteoarthritis, psoriasis, acute respiratory disease syndrome, inflammatory bowel disease, multiple sclerosis, joint inflammation, reactive arthritis, hay fever, atherosclerosis, rheumatoid arthritis, bursitis, gouty arthritis, osteoarthritis, polymyalgia rheumatic arthritis, septic arthritis, infectious arthritis, asthma, autoimmune diseases, chronic inflammation, chronic prostatitis, glomerulonephritis, nephritis, inflammatory bowel diseases, pelvic inflammatory disease, reperfusion injury, transplant rejection, vasculitis, myocarditis, colitis, appendicitis, peptic ulcer, gastric ulcer, duodenal ulcer, peritonitis, pancreatitis, ulcerative colitis, seudomembranous colitis, acute colitis, ischemic colitis, diverticulitis, epiglottitis, achalasia, cholangitis, cholecystitits, hepatitis, Crohn's disease, enteritis, Whipple's disease, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, pneumonitits, pneumoultramicroscopic silicovolcanoconiosis, alvealitis, bronchiolitis, pharyngitis, pleurisy, sinusitis, influenza, respiratory syncytial virus infection, HIV infection, hepatitis B virus infection, hepatitis C virus infection, herpes virus infection disseminated bacteremia, Dengue fever, candidiasis, malaria, filariasis, amebiasis, hydatidcysts, burns, dermatitis, dermatomyositis, sunburn, urticaria, Warts, Wheals, vasulitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, adult respiratory distress syndrome, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thyroiditis, systemic lupus erythematosis, Goodpasture's syndrome, Behcet's syndrome, allograft rejection, graft-versus-host disease, Type I diabetes, Type II diabetes, ankylosing spondylitis, Berger's disease, Reiter's syndrome, Hodgkin's disease, ileus, hypertension, irritable bowel syndrome, myocardial infarction, sleeplessness, anxiety, local inflammation, or stent thrombosis. In some embodiments, the disease is caused by infection by a microorganism, e.g., Staphylococcus aureus, Streptococcus, Streptococcus pneumonia, Neisseria gonorrhoeae, Mycobacterium tuberculosis, Borrelia burgdorferi, or Haemophilus influenza.

In some embodiments, the composition is targeted to macrophages of the subject (e.g., the composition is phagocytized by the macrophage). In some embodiments, the composition has a biological effect in the subject (e.g., reduction of inflammation). In some embodiments, the administering is parentally, via inhalation, or nasally. For example, in some embodiments, the administering is to the lung via inhaler or nebulizer.

Additional embodiments provide the use of any of the aforementioned compositions to treat a disease in a subject.

Still other embodiments provide the use of any of the aforementioned compositions for the manufacture of a medicament or for use in medicine.

Additional embodiments are described herein.

### DESCRIPTION OF THE FIGURES

FIG. 1 shows an asymmetic unit for a) CFZ-HCl (form B), b) CFZ-2HCl, and c) CFZ-HCl-2MeOH determined by single crystal X-ray diffraction.

FIG. 2 shows a synthesis scheme showing the crystallization solution and crystallization method (glass vial or microfluidic platform) for exemplary CFZ solid forms.

FIG. 3 shows chemical characterization of CFZ. Chemical structure of Clofazimine (CFZ) with its two protonation sites and corresponding pKa values of 2.31 and 9.29 (A). Illustration of the cellular accumulation and stabilization of a free base (B), versus protonated (BH⁺), versus salt (BHCl), forms of the drug upon ion-ion interaction of BH+and cellular Cl-depends on the drug's pKa and cellular pH and Cl levels (B). CFZ-HCl solubility-pH study revealed the solution pH and chloride dependence of the stabilization of the free base versus salt form of the drug with respect to its solubility parameters; intrinsic solubility (So), apparent pKa (pKa′), pHmax and corresponding salt solubility product (C).

FIG. 4 shows CFZ-HCl fluorescence.

FIG. 5 shows model and simulation of the effects of V-ATPase and CLC7 on the lysosomal accumulation of CFZ-HCl. V-ATPase inhibition showed more significant effect than CLC7 inhibition on the accumulation of CFZ-HCl at the rate of 0.01 picomol/cell/day, as reflected by the changes in the lysosomal pH, Cl, and membrane potential values of the CFZ-HCl containing lysosome from that of the CFZ-HCl free lysosome (A). V-ATPase inhibition showed more significant effect than CLC7 inhibition on the physiological accumulation of CFZ-HCl at the rate of 0.01 picomol/cell/day, as reflected by the changes in the lysosomal pH, Cl, and membrane potential values of the CFZ-HCl containing lysosome from respective baseline physiological values (B). Arrow signs represent values outside of the axes plot range.

FIG. 6 shows model and simulation of the effects of V-ATPase and CLC7 on the lysosomal accumulation of CFZ-HCl at higher dose. V-ATPase inhibition showed more significant effect than CLC7 inhibition on the accumulation of CFZ-HCl at the rate of 0.1 picomol/cell/day, as reflected by the changes in the lysosomal pH, Cl, and membrane potential values of the CFZ-HCl containing lysosome from that of the CFZ-HCl free lysosome (A). V-ATPase inhibition showed more significant effect than CLC7 inhibition on the physiological accumulation of CFZ-HCl at the rate of 0.1 picomol/cell/day, as reflected by the changes in the lysosomal pH, Cl, and membrane potential values of the CFZ-HCl containing lysosome from respective baseline physiological values (B).

FIG. 7 shows model and simulation of the effects of V-ATPase and cytoplasmic chloride on the lysosomal accumulation of CFZ-HCl. V-ATPase inhibition showed more significant effect than cytoplasmic chloride inhibition on the accumulation of CFZ-HCl at the rate of 0.01 picomol/cell/day, as reflected by the changes in the lysosomal pH, Cl, and membrane potential values of the CFZ-HCl containing lysosome from that of the CFZ-HCl free lysosome (A). V-ATPase inhibition generally showed more significant effect than cytoplasmic chloride inhibition although the simultaneous inhibition of both parameters showed even more significant effect on the physiological accumulation of CFZ-HCl at the rate of 0.01 picomol/cell/day, as reflected by the changes in the lysosomal pH, Cl, and membrane potential values of the CFZ-HCl containing lysosome from respective baseline physiological values (B).

FIG. 8 shows model and simulation of the effects of V-ATPase and cytoplasmic chloride on the lysosomal accumulation of CFZ-HCl at higher dose. V-ATPase inhibition showed more significant effect than cytoplasmic chloride inhibition on the accumulation of CFZ-HCl at the rate of 0.1 picomol/cell/day, as reflected by the changes in the lysosomal pH, Cl, and membrane potential values of the CFZ-HCl containing lysosome from that of the CFZ-HCl free lysosome (A). V-ATPase inhibition generally showed more significant effect than cytoplasmic chloride inhibition although the simultaneous inhibition of both parameters showed even more significant effect on the physiological accumulation of CFZ-HCl at the rate of 0.1 picomol/cell/day, as reflected by the changes in the lysosomal pH, Cl, and membrane potential values of the CFZ-HCl containing lysosome from respective baseline physiological values (B). Arrow signs represent values outside of the axes plot range.

FIG. 9 shows cell viability upon exposure to different inhibitors.

FIG. 10 shows intracellular CFZ accumulation.

FIG. 11 shows that clodronate inhibits CLDI accumulation in peritoneal macrophages.

FIG. 12 shows that clodronate did not significantly alter CLDI accumulation in lung macrophages.

FIG. 13 shows the percentage of CLDI (+) cells in peritoneal and alveolar lavage (*=p<0.05

FIG. 14 shows that clodronate reduces Kupffer cells and CLDI accumulation in the liver.

FIG. 15 shows that quantitative cytometric analysis of liver cryosections reveals a reduction in F4/80 macrophages and CLDI accumulation.

FIG. 16 shows that clodronate reduces red-pulp macrophages and CLDI accumulation in the spleen.

FIG. 17 shows that quantitative cytometric analysis of spleen cryosections reveals a reduction in F4/80 macrophages and CLDI accumulation.

FIG. 18 shows that the total amount of drug sequestered is significantly reduced by macrophage depletion.

FIG. 19 shows that CLDIs dissolve at significantly higher rate in absence of macrophages.

FIG. 20 shows peritoneal lavage 48 hours post-CLDI injection. Left panel is brightfield image, and right panel is Cy5 fluorescence.

FIG. 21 shows exponential regression analysis on recovered CFZ (n=3 mice per data point).

FIG. 22 shows modeling of the effects of drugs on lysosomal ion homeostasis. Physiological framework used to model lysosomal ion transport (A). The V-ATPase actively pumps protons into the lysosome (1) while the proton-chloride antiporter CLC7 dissipates the ensuing increase in membrane potential by coupling the efflux of protons with the influx of chloride (2). Protons escape the lysosomes through diffusion across the lysosomal membrane (3), and protons in the lysosome are sequestered through the buffering capacity of resident lysosomal components (4). Free protons that accumulate in the lysosomal lumen contribute to the decrease in lysosomal pH. Based on this mechanism, the effect of drugs was captured by modeling different lysosomal shapes and volumes (B). Concomitantly, the drug-dependent inhibition of V-ATPase function was modeled by varying the proton pumping activity (C); the drug-dependent change in membrane permeability was modeled by varying the proton leak from the lysosome (D); and, the drug-dependent perturbation of membrane potential regulation was modeled by inhibiting CLC-7 (E) or decreasing cytoplasmic chloride (F).

FIG. 23 shows the effect of individual lysosomal ion stressor on spherical versus tubular lysosomal physiology. (A) The effect of inhibiting the number of V-ATPase molecule per lysosome showed significant changes in lysosomal pH and Cl, with minimal change in membrane potential in both spherical and tubular lysosomes. (B) The effect of increasing proton-specific membrane permeability per lysosome showed significant changes in lysosomal pH and Cl, with minimal change in membrane potential in both spherical and tubular lysosomes.

FIG. 24 shows effects of lysosomal surface expansion and associated tolerance on lysosomal physiology. (A) Simultaneous increment in lysosomal radius and surface area, with a constant volume of 1.65×10⁻¹⁶ L induced progressive perturbation in lysosomal pH, Cl and membrane potential. (B) Individual effects of varying V-ATPase number and membrane proton permeability on the lysosomal physiology of surface area expansion-mediated lysosomal stress.

FIG. 25 shows the effect of combination of lysosomal ion stressors in spherical versus tubular lysosomes. (A) Dimensions of tubular and spherical lysosomes. (B) The simultaneous inhibition of V-ATPase and CLC7 numbers induced changes in both spherical and tubular lysosomes with minimal difference between the two lysosomal morphologies. (C) The simultaneous inhibition of V-ATPase and membrane proton permeabilization induced very similar and significant changes in the overall physiology of both spherical and tubular lysosomes.

FIG. 26 shows the effect of the simultaneous inhibition of the transport of chloride and proton ions in spherical versus tubular lysosomes. (A) Dimensions of tubular and spherical lysosomes. (B) The simultaneous inhibition of the cytoplasmic chloride and V-ATPase number per lysosome induced significant changes in lysosomal pH, Cl, and membrane potential.

FIG. 27 shows the effect of simultaneous inhibition of proton and chloride transport in spherical versus various sized disc-shaped lysosomes.

FIG. 28 shows the effect of simultaneous V-ATPase inhibition and membrane permeabilization in spherical versus various sized disc-shaped lysosomes.

FIG. 29 shows the effect of individual lysosomal chloride transportation stressors on the physiology of spherical versus tubular lysosomes. (A) Modeling the effect of varying CLC7 number on lysosomal pH, Cl, and membrane potential with respect to different lysosomal morphology. (B) Modeling the effect of varying cytoplasmic chloride concentration on lysosomal pH, Cl, and membrane potential with respect to different lysosomal morphology.

FIG. 30 shows the effect of simultaneous CLC7 inhibition and membrane proton permeabilization on the physiology of spherical versus tubular lysosomes. (A) Lysosomal dimensions used to generate spherical and tubular lysosomes. (B) Modeling the effect of simultaneous variations of CLC7 number and membrane proton permeability on lysosomal pH, Cl, and membrane potential.

FIG. 31 shows the effect of the simultaneous cytoplasmic chloride inhibition and membrane proton permeabilization on spherical versus disc-shaped lysosomal physiology.

FIG. 32 shows A) intracellular, lysosomal hydrochloride transport and B) how this transport leads to stabilization of an physiologically insoluble weakly basic, hydrochloride drug salt.

### DEFINITIONS

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human or non-human mammal subject.

As used herein, the term “diagnosed,” as used herein, refers to the recognition of a disease by its signs and symptoms (e.g., resistance to conventional therapies), or genetic analysis, pathological analysis, histological analysis, and the like.

As used herein, the term “effective amount” refers to the amount of a compound (e.g., a compound of the present disclosure) sufficient to effect beneficial or desired results. An effective amount can be administered in one or more administrations, applications or dosages and is not limited to a particular formulation or administration route.

As used herein, the term “co-administration” refers to the administration of at least two agent(s) (e.g., a compound of the present disclosure) or therapies to a subject. In some embodiments, the co-administration of two or more agents/therapies is concurrent. In some embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

As used herein, the term “pharmaceutical composition” refers to the combination of an active agent with a carrier, inert or active, making the composition especially suitable for diagnostic or therapeutic use in vivo, in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa., (1975)).

As used herein, the term “physiologically insoluble form” is used in its broadest sense, to describe a non-aqueous (e.g. solid, liquid or gel) phase of a drug, that equilibrates with freely dissolved drug molecules in a surrounding aqueous phase (e.g., within a living system) at a concentration lower than the freely soluble concentration of the drug when the drug form is allowed to equilibrate with pure water.

As used herein, the term “cell stabilizing agent” refers to an agent (e.g., ion, lipid, or other agent) in complex with a biocrystalline mimetic of pharmaceutical agent. In some embodiments, the cell stabilizing agent stabilizes the complex in vivo.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water and industrial samples. Such examples are not however to be construed as limiting the sample types applicable to the present disclosure.

As used herein, the terms “purified” or “to purify” refer, to the removal of undesired components from a sample. As used herein, the term “substantially purified” refers to molecules that are at least 60% free, at least 65% free, at least 70% free, at least 75% free, at least 80% free, at least 85% free, at least 90% free, at least 95% free, at least 96% free, at least 97% free, at least 98% free, at least 99% free, or 100% free from other components with which they usually associated.

As used herein, the term “modulate” refers to the activity of a compound (e.g., a compound of the present disclosure) to affect (e.g., to promote or retard) an aspect of cellular function.

As used herein, the phrase “in need thereof” means that the subject has been identified as having a need for the particular method or treatment. In some embodiments, the identification can be by any means of diagnosis. In any of the methods and treatments described herein, the subject can be in need thereof. In some embodiments, the subject is in an environment or will be traveling to an environment in which a particular disease, disorder, condition, or injury is prevalent.

### DETAILED DESCRIPTION

Provided herein are physiologically insoluble forms of drugs. In particular, provided herein are physiologically insoluble salts of drugs (e.g., basic drugs) and their use in treatment of disease.

The elucidation of molecular mechanisms influencing the solubility of poorly soluble chemical agents in different cells, tissues and organs of mammals is interesting from a fundamental chemical and biological perspective. Cells are able to eliminate soluble chemical agents via metabolism and facilitated or active transport across the plasma membrane. Moreover, in the case of foreign, insoluble particles, phagocytic cells of the immune system are especially equipped to actively ingest these particles and isolate them from the rest of the organism. However, for poorly soluble compounds, that can exist both as soluble and physiologically insoluble forms within the cells of an organism, the mechanisms controlling the bioaccumulation and distribution of soluble and physiologically insoluble forms of these agents in the different cells and organs of the body are not known.

Many FDA-approved drugs (e.g., clofazimine, amiodarone, azithromycin, chloroquine, gefitinib) fall within the class of poorly soluble compounds that are actively sequestered within macrophages (Ohkuma, et al., J. Cell Biol. 1981, 90, 656-664; Poole, et al., J. Cell Biol. 1981, 90, 665-669; Maxfield, et al., J. Cell Biol. 1982, 95, 676-681; Quaglino, et al., Am. J. Physiol. Lung Cell. Mol. Physiol. 2004, 287, 438-447; Bergman, et al., Int. J. Pharm. 2007, 341, 134-142; Gladue, et al., Antimicrob. Agents Chemother. 1989, 33, 277-282.). Elucidating the mechanisms affecting their solubility and accumulation inside cells is relevant to understanding drug toxicity, disposition and efficacy (Fu, et al., Nat. Chem. 2014, 6, 614-622). Particularly, weakly basic molecules have been implicated to be accumulated via the lysosomal pathway in macrophages due to the decrease in pH of the vesicles that contain the drug (Ohkuma et al., supra). Research into mechanisms responsible for in vivo drug bioaccumulation and retention has been very sparse.

Macrophages (MΦs) are critical immune cells vital for mammalian self-nonself recognition and immunological homeostasis, particularly for resolving the inflammatory response in multiple disease states. Recent evidence also indicates that dysregulation in MΦ signaling responses can lead to inherently deleterious effects on the host with pathological consequences resulting in arthritis, tumor growth and metastasis, atherosclerotic plaque formation, diabetes and other disease conditions. Certain orally bioavailable drugs (e.g., CFZ) have been found to massively bioaccumulate in MΦs to the point that they precipitate out, forming physiologically insoluble intracellular drug inclusions. These precipitates (crystal-like-drug-inclusions (CLDIs) or CFZ Biocrystals) have been characterized to contain Clofazimine Hydrochloride (CFZ-HCl) crystalline domains.

Macrophage (MΦ)-targeted drugs and bioimaging contrast agents offer great promise for the diagnosis and treatment of inflammatory diseases, infections and cancers. Beyond simple diagnosis, theranosis the combination of diagnosis and therapy is also being sought as a means to develop highly personalized therapeutic approaches. The ability to formulate stable, MΦ-targeted formulations that elicit sustained anti-inflammatory effects in a localized or specific manner is beneficial in terms of avoiding unwanted interaction of therapeutic agents in non-diseased sites.

Accordingly, provided herein are polymorph and various solid forms of anti-inflammatory MΦ-targeted, physiologically insoluble drug formulations and the symbiotic mechanisms that stabilize the crystalline polymorphs and the cells themselves. In particular, provided herein are the a) structure and design of physiologically insoluble HCl salts of weakly basic drugs and the b) interdependent role of lysosomal morphology, V-ATPase activity, chloride transport, membrane proton permeability and MΦ viability in the intracellular accumulation and stabilization of CFZ-HCl particulate accumulation despite increased intrinsic solubility of CFZ-H+ relative to CFZ. Such stabilization mechanisms and structural packing of drug crystals within MΦs are innovative means of loading cells with massive amounts of drugs. Consequently, since these are stable, MΦ-targeted drug crystals, their effect is sustained for a long period of time without the need for repeated dosages and their effect is localized to the site of therapeutic need thereby reducing any systemic drug side effects.

### I. Compositions

As described above, the present disclosure contemplates physiologically insoluble HCl salts of weakly basic molecules (e.g., drugs). Physiologically insoluble forms of active agents are formed using any suitable method. For example, in some embodiments, the active agent is dissolved in a solvent (e.g., methanol) and equal volumes of anti-solvents (e.g., comprising counterion) are added to obtain physiologically insoluble forms. The supernatant is then removed, the aggregates (e.g., crystals) are washed, and optionally lyophilized.

In some embodiments, the present disclosure is exemplified with CFZ, although the present disclosure is not intended to be limited to CFZ. CFZ (Harbeck, et al., Ann. Pharmacother. 1999, 33, 250; Levy, L. Am. J. Trop. Med. Hyg. 1974, 23, 1097-1109; Aplin, et al., Experientia 1975, 31, 468-469; McDougall, et al., Br. J. Dermatol. 1980, 102, 227-230; McDougall, Int J Lepr. Other Mycobact. Dis. 1974, 42, 1-12) is an FDA-approved riminophenazine antibiotic that has been remarkably effective against mycobacterial infections such as leprosy (Tolentino, et al., Int J Lepr. Other Mycobact. Dis. 1974, 42, 416-418; Karuru, et al. Lepr. Rev. 1970, 41, 83-88; Leiker, et al., Lepr. Rev. 1971, 42, 125-130) and mycobacterium avium infections in AIDS patients (Rensburg, et al., Antimicrob. Agents Chemother. 1992, 36, 2729-2735; Reddy, et al., J. Antimicrob. Chemother. 1999, 43, 615-623). It has also attracted attention because of its anti-inflammatory (Cholo, et al., J. Antimicrob. Chemother. 2012, 67, 290-298; Helmy, et al., Lepr. Rev. 1972, 42, 162-177) and immunomodulatory (Ren, et al., PLoS One 2008, 3, e4009) properties, and it is especially used for treating leprotic skin inflammations (erythema leprosum nodosum) (Cholo et al., supra; Helmy et al., supra; Barry, et al., Lepr. Rev. 1965, 36, 3-7; Barry, et al., Nature 1957, 179, 1013-1015).

The chemical structure of CFZ (3-(p-chloroanilino)-10-(p-chlorophenyl)-2, 10-dihydro-2-isopropyliminophenazine) is

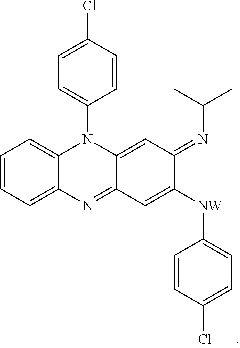

Derivatives of CFZ are described, for example, in Franzblau et al., Antimicrob Agents Chemother 1988;32:1583-5; Jagannath C, et al., Am J Respir Crit Care Med 1995;151:1083-6; O′Sullivan JF, et al., Health Cooperation Papers 1992;12:191-7; and O'Connor R, et al., J Chromatogr B Biomed Appl 1996;681:307-15; each of which is herein incorporated by reference in its entirety.

When dissolved in pure water, CFZ-HCl exhibits greater solubility than the unprotonated CFZ free base. However, when solid CFZ-HCl micro or nanoparticles are ingested by macrophages, the presence of a concentrative hydrochloride pumping mechanism in the lysosomes serves to stabilize the CFZ-HCl in solid form. Polymorphs and different solid forms of a drug substance are known to exhibit different physical and chemical properties, such as, for example, solubility, bioavailability, color, crystal habit, etc. The polymorph and solid forms described herein provide a therapeutic with enhanced onset time, greater bioavailability, or greater stability within the system.

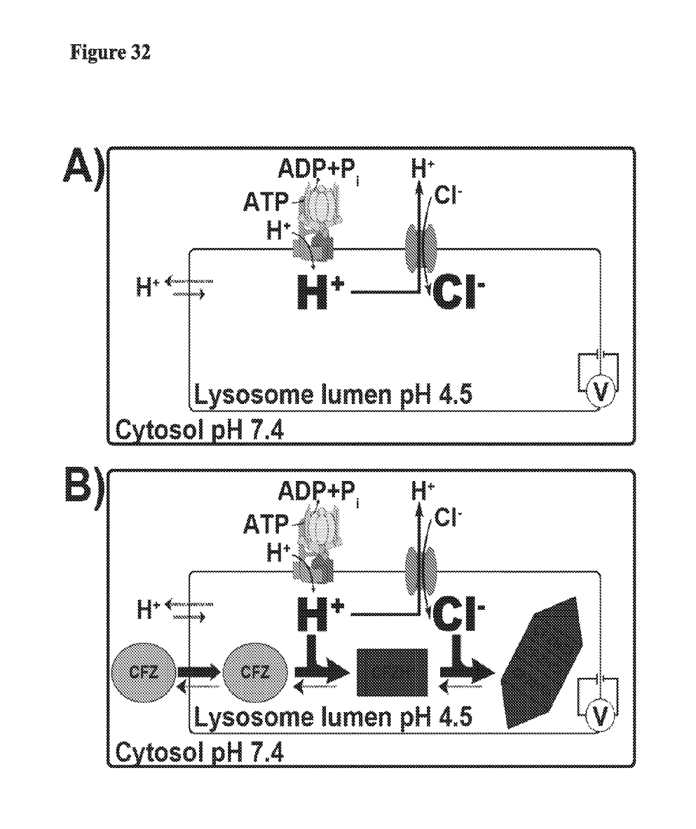

FIG. 32 shows intracellular, lysosomal hydrochloride transport and stabilization of physiologically insoluble HCl salts of weakly basic drugs in lysosomes. The present disclosure is not limited to particular conformations of such physiologically insoluble HCl salts. In some embodiments, the salt is protonate, membrane-impermeant, crystalline, particulate or aggregated. In some embodiments, the pH max of the drug is higher than the lysosomal pH.

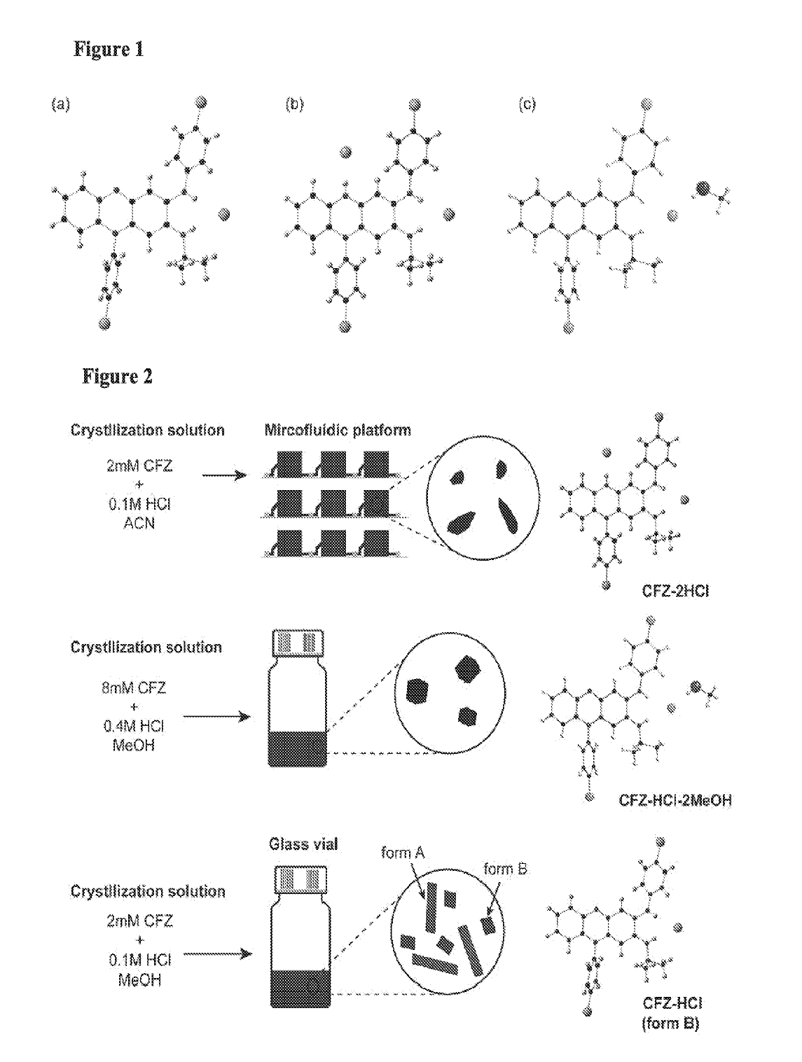

In some embodiments, the physiologically insoluble salts of the present disclosure are exemplified with crystalline salts of CFZ, although the disclosure is not limited to cystalline forms of molecules. FIGS. 1 and 2 and Table 1 below describe crystalline forms of CFZ (e.g., CFZ-HCl with one CFZ and one HCl (CFZ-HCl form B) per asymmetric unit arranged in a triclinic space group with two asymmetric units per unit cell, one CFZ and two HCl (CFZ-2HCl) per asymmetric unit arranged in a monoclinic space group with 4 asymmetric units per unit cell; or a solvate with one CFZ, one HCl and two methanol molecules per asymmetric unit arranged in an orthorhombic space group with 8 asymmetric units per unit cell). Only one of the MeOH molecules are shown in the asymmetric unit of CFZ-HCl-2MeOH in FIG. 1.

The present disclosure is not limited to the crystal forms described herein. Additional polymorphs with different solvates (e.g., inorganic or organic) are specifically contemplated.

The present disclosure is not limited to CFZ. The present disclosure specifically contemplates physiologically insoluble salts of other weakly basic drugs with high affinity with chloride (Roy and Flynn, Pharmaceutical Research 6:147 1989). In some embodiments, the methods described in Roy and Flynn (supra) are used to identify other specific solids formed by a weakly basic molecule (given by the shape of the pH-solubility curve and its measured pHmax, in absolute terms) with “high” affinity for chloride (given by the measured Ksp in absolute terms) and low solubility (given by the measured intrinsic solubility of the protonated drug at pHmax in absolute terms). Table 2 shows exemplary drugs suitable for formulation as described herein.

The compositions described herein have the benefits of reduced cellular toxicity, reduced systemic toxicity, reduced side effects, sustained controlled release delivery, and use as non-invasive diagnostics.

In some embodiments, the methods described in the experimental section and below are used to screen and characterize physiologically insoluble salts of additional drugs. It is further contemplated that lysosomes of various cell types, macrophages in particular, accommodate the conversion of a free base form of the drug to a hydrochloride salt form of the drug dictated by thermodynamics law of mass action. In some embodiments, in case of cellular internalization of the salt form of the drug itself, the thermodynamic equilibrium between the solid salt and soluble salt forms of the drug is established according to the pHmax and Ksp values of the hydrochloride drug salt and the relationship of these parameters to the cellular pH and chloride content in a manner that does not perturb cellular physiology.

Experiments described herein utilized a lysosomal ion regulation model comprising a spherical lysosomal vesicle with radius of 0.34 um, lysosomal proteins such as the proton pumping Vacuolar ATPase (V-ATPase), chloride transporter (CLC7), membrane proton leak, as well as ions. To represent the lysosomal hydrochloride drug salt accumulation in the model, a range of CFZ accumulation rates in RAW 264.7 Cells, a macrophage cell line was used and set to equal the dose-dependent rates by which proton and chloride ions get sequestered by CFZ. This allowed the model to determine a) the effect of introducing a proton and chloride sequestering drug into the lysosome on lysosomal pH, chloride, and membrane potential; and b) the accommodative capability of the lysosome of hydrochloride drug salt by monitoring the changes in the lysosomal pH, chloride, and membrane potential from their respective physiological values. As such, other drug candidates can be similarly characterized for their accumulation rates, inhibited state accumulation as well as overall macrophage-driven stability.

In some embodiments, candidate drug salts are screened for stability by constructing a pH dependent solubility profile in aqueous media. Such a study was first established by Kramer and Flynn in 1972 to help characterize the relationship between the stabilization of free base versus salt form of a given drug molecule and the environmental pH. In some embodiments, in order to characterize the stabilizing role of macrophages on synthetic formulations in vivo, in vivo experiments are performed. In some embodiments, using mice as an experimental model, animals are treated with liposomal formulations of clodronate to selectively kill different macrophage populations, or with liposomal formulations of phosphate-buffered saline, to serve as a control. Following the depletion of macrophages, mice are treated with various synthetic drugs salts through a variety of routes of administration (intraperitoneal, intravenous, inhalation, etc.) depending on the specifics of the formulation. Following treatment with the synthetic formulations, the role of macrophages in stabilizing these formulations is established through the use of microscopic imaging and biochemical analysis of tissues. Using microscopic analysis, the presence of the physiologically insoluble drug aggregate in the tissues or macrophage populations is detected and quantified via fluorescence microscopy. Biochemically, the stabilizing role of macrophages on formulations is quantified by determining the concentration of drug found within various organs (liver, spleen, lung), as well as systemically by measuring the concentration of drug within the blood. Other drug candidates are similarly studied also via absorbance spectroscopy or HPLC analysis.

In some embodiments, in order to characterize the stabilizing role of macrophages on synthetic formulations ex vivo, different macrophages are collected from mice, such as peritoneal, alveolar, and Kupffer cells of the liver. Once collected, the stabilizing role of the macrophage is determined through the use of various pharmacological agents which interfere with cellular acidification processes, or which kill the cell itself. The proton-pump Vacuolar-type proton-ATPase (V-ATPase) is inhibited through treatment with bafilomycin A1, resulting in a more alkaline endo-lysosomal environment. Cells are killed by treatment with staurosporine, which induces apoptosis within the cell. Following treatment with the pharmacologic agent, macrophages are exposed to the various synthetic formulations and the stability of the formulations within the cellular environment is monitored. The internalization and stabilization is qualitatively monitored using microscopy, and can be quantified by measuring the total soluble drug released into the extracellular media at various time points throughout the experiment.

In some embodiments, various drug polymorph formulations are tested for their response in an inflammatory environment in small animal models (rodents). Examples include, but are not limited to, carrageenan-induced footpad or joint inflammation, monosodium urate-induced inflammation in the joints, lipopolysaccharide-induced inflammation in the lungs, airpouch models to study local inflammation responses etc. The full spectra of characterization on inflammatory response and treatment can be performed via common biomarkers analysis via immunohistochemistry (for cellular proliferation density and cell-types), western blots (for modification of common proteins and phosphorylation events) and ELISAs (for cytokine/chemokine analysis such as TNFα, IL-1β and IL-IRA). Drug stability can be measured via 1H-NMR or HPLC of local tissue digests in these environments.

TABLE 1

| | CFZ-HCl | | |
|---|---|---|---|
| | (form B) | CFZ-2HCl | CFZ-Cl-2MeOH |
| chemical | C₂₇H₂₃Cl₃N₄ | C₂₇H_{23.76}Cl_{3.76}N₄ | C₂₉H₃₁Cl₃N₄O₂ |
| formula | | | |
| formula weight | 509.84 | 537.46 | 573.93 |
| T (K) | 100 | 100 | 100 |
| crystal system | triclinic | monoclinic | orthorhombic |
| space group | P-1 | P2₁/c | Pbcn |
| a (Å) | 8.754 | 12.073 | 14.151 |
| b (Å) | 12.178 | 15.270 | 13.796 |
| c (Å) | 12.589 | 14.612 | 28.103 |
| α (deg) | 63.841 | 90 | 90 |
| β (deg) | 80.740 | 111.04 | 90 |
| γ (deg) | 83.940 | 90 | 90 |
| volume (A³) | 1188.0 | 2514.20 | 5486.2 |
| D_{calcd} (g cm⁻³) | 1.425 | 1.420 | 1.390 |
| Z | 2 | 4 | 8 |
| R₁ | 0.0331 | 0.0364 | 0.0319 |
| wR₂ | 0.0760 | 0.0816 | 0.0823 |
| GOF | 1.074 | 1.052 | 1.040 |
| Completeness | 99.4% | 99.8% | 99.9% |
| Color | dark red | purple | red |

TABLE 2
(Vd in L/Kg unit)

| | | | | | |
|---|---|---|---|---|---|
| Raloxifene | 2348 | Irinotecan | 15.2 | Atorvastatin | 5.4 |
| Doxorubicin | 682 | Donepezil | 14 | Clozapine | 5.4 |
| Solifenacin | 671 | Triameterene | 13.4 | Buspirone | 5.3 |
| Hydroxychloroquine | 525 | Promethazine | 13.4 | Isotretinoin | 5 |
| Chloroquine | 196.5 | Citalopram | 12.3 | Verapamil | 5 |
| Loratadine | 120 | Sirolimus | 12 | Flecainide | 4.9 |
| Mitoxantrone | 90 | Posaconazole | 11.9 | Aripiprazole | 4.9 |
| Vinorelbine | 76 | Fluphenazine | 11 | Mirtazapine | 4.5 |
| Amiodarone | 66 | Memantine | 10.9 | Cyclosporine | 4.5 |
| Tamoxifen | 55 | Itraconazole | 10.7 | Diphenhydramine | 4.5 |
| Toremifene | 48.38 | Spironolactone | 10 | Meperidine | 4.4 |
| Fluoxetine | 35 | Felodipine | 10 | Propranolol | 4.3 |
| Azithromycin | 31 | Quetiapine | 10 | Metoprolol | 4.2 |
| Capecitabine | 27.27 | Ivermectin | 9.91 | Fulvestrant | 4.15 |
| Loratadine | 26 | Vincristine | 9.79 | Sunitinib | 4 |
| Idarubicin | 24.7 | Praziquantel | 9.55 | Fentanyl | 4 |
| Doxepin | 24 | Ribavirin | 9.3 | Mycophenolate | 3.8 |
| Chlorpromazine | 21 | Paliperidone | 9.1 | Methadone | 3.6 |
| Hydroxyzine | 19 | Dronabinol | 8.9 | Emtricitabine | 3.5 |
| Mefloquine | 19 | Desvenlafaxine | 7.5 | Riluzole | 3.4 |
| Bupropion | 18.6 | Ropinirole | 7.5 | Metoclopramide | 3.4 |
| Nortriptyline | 18 | Pramipexole | 7.3 | Diltiazem | 3.3 |
| Haloperidol | 18 | Docetaxel | 7.27 | Temsirolimus | 3.3 |
| Cinacalcet | 17.6 | Tigecycline | 7.2 | Morphine | 3.3 |
| Paroxetine | 17 | Duloxetine | 7 | Nitroglycerin | 3.3 |
| Olanzapine | 16.4 | Imatinib | 6.2 | Alfuzosin | 3.2 |
| Naltrexone | 16.1 | Varenicline | 6.2 | Clonazepam | 3.2 |
| Amlodipine | 16 | Celecoxib | 6.12 | Clonazepam | 3.2 |
| Hydroxyzine | 16 | Dextroamphetamine | 6.11 | Chlorpheniramine | 3.2 |
| Citalopram | 15.4 | Ibandronate | 5.8 | Nilotinib | 3.0 |

In some embodiments, compositions further comprise one or more lipids. In some embodiments, the lipids are present as a liposome that encapsulates the pharmaceutical agent (e.g., to mimic cellular membranes). For example, in some embodiments, biomimetic forms of the crystal are formed using a remote loading of drugs via ammonium salt method or direct lipid encapsulation of ammonium salt precipitated crystal salt of drug (See e.g., Ceh et al., Langmuir, 1995, 11 (9), pp 3356-3368).

In some embodiments, the pharmaceutical agent is encapsulated in a niosome (See e.g., Moghassemi et al, Journal of Controlled Release, Volume 185, 10 July 2014, Pages 22-36). Niosomes are a class of molecular cluster formed by self-association of non-ionic surfactants in an aqueous phase.

In some embodiments, the lipid (e.g., phospholipid) structures surrounding crystalline pharmaceutical agent is tailored to the target organ/tissue lipid composition. For example, the natural lipid composition in the lungs is rich in choline lipids. Hence, in some embodiments, synthetic lipids comprising phosphatidylcholines are used in formulating compositions for delivery to the lung. The specific composition and morphology of the formulation is modulated through temperature and concentrations of lipid as well as drug:lipid ratio (See e.g., Keswani et al., Mol Pharm. 2013 May 6;10(5):1725-35.; herein incorporated by reference in its entirety).

In some embodiments, lipids are functionalized to aid in phagocytosis by macrophages. For example, in some embodiments, compositions are enveloped with mannose-conjugated phospholipids that are internalized via the CD206/CD205 receptor on macrophages.

In some embodiments, at least one (or some) of the lipids is/are amphipathic lipids, defined as having a hydrophilic and a hydrophobic portion (typically a hydrophilic head and a hydrophobic tail). The hydrophobic portion typically orients into a hydrophobic phase (e.g., within the bilayer), while the hydrophilic portion typically orients toward the aqueous phase (e.g., outside the bilayer, and possibly between adjacent apposed bilayer surfaces). The hydrophilic portion may comprise polar or charged groups such as carbohydrates, phosphate, carboxylic, sulfato, amino, sulfhydryl, nitro, hydroxy and other like groups. The hydrophobic portion may comprise apolar groups that include without limitation long chain saturated and unsaturated aliphatic hydrocarbon groups and groups substituted by one or more aromatic, cyclo-aliphatic or heterocyclic group(s). Examples of amphipathic compounds include, but are not limited to, phospholipids, aminolipids and sphingolipids.

In some embodiments, the lipids are phospholipids. Phospholipids include without limitation phosphatidylcholine, phosphatidylethanolamine, phosphatidylglycerol, phosphatidylinositol, phosphatidylserine, and the like. It is to be understood that other lipid membrane components, such as cholesterol, sphingomyelin, cardiolipin, etc. may be used.

The lipids may be anionic and neutral (including zwitterionic and polar) lipids including anionic and neutral phospholipids. Neutral lipids exist in an uncharged or neutral zwitterionic form at a selected pH. At physiological pH, such lipids include, for example, dioleoylphosphatidylglycerol (DOPG), di acylphosphatidylcholine, diacylphosphatidylethanolamine, ceramide, sphingomyelin, cephalin, cholesterol, cerebrosides and diacylglycerols. Examples of zwitterionic lipids include without limitation dioleoylphosphatidylcholine (DOPC), dimyristoylphosphatidylcholine (DMPC), and dioleoylphosphatidylserine (DOPS). An anionic lipid is a lipid that is negatively charged at physiological pH. These lipids include without limitation phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

Collectively, anionic and neutral lipids are referred to herein as non-cationic lipids. Such lipids may contain phosphorus but they are not so limited. Examples of non-cationic lipids include lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palmitoyloleoyl-phosphatidylethanolamine (POPE) palmitoyloleoylphosphatidylcholine (POPC), egg phosphatidylcholine (EPC), di stearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), phosphatidylserine, phosphatidylinositol, sphingomyelin, cephalin, cardiolipin, phosphatidic acid, cerebrosides, dicetylphosphate, and cholesterol.

Additional nonphosphorous containing lipids include stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, dioctadecyldimethyl ammonium bromide and the like, diacylphosphatidylcholine, diacylphosphatidylethanolamine, ceramide, sphingomyelin, cephalin, and cerebrosides. Lipids such as lysophosphatidylcholine and lysophosphatidylethanolamine may be used in some instances. Noncationic lipids also include polyethylene glycol-based polymers such as PEG 2000, PEG 5000 and polyethylene glycol conjugated to phospholipids or to ceramides (referred to as PEG-Cer).

In some embodiments, lipids are cationic lipids (e.g., those described herein).

In some instances, modified forms of lipids may be used including forms modified with detectable labels such as fluorophores. In some instances, the lipid is a lipid analog that emits signal (e.g., a fluorescent signal). Examples include without limitation 1,1′-dioctadecyl-3,3,3′,3′-tetramethylindotricarbocyanine iodide (DiR) and 1,1′-dioctadecyl-3,3,3′,3′-tetramethylindodicarbocyanine (DiD).

In some embodiments, the lipids are biodegradable in order to allow release of encapsulated agent in vivo and/or in vitro. Biodegradable lipids include but are not limited to 1,2-dioleoyl-sn-glycero-3-phosphocholine (dioleoyl-phosphocholine, DOPC), anionic 1,2-di-(9Z-octadecenoyl)-sn-glycero-3-phospho-(1′-rac-glycerol) (dioleoyl-phosphoglycerol, DOPG), and 1,2-distearoyl-sn-glycero-3-phosphoethanolamine(distearoyl-phosphoethanolamine, DSPE). Non-lipid membrane components such as cholesterol may also be incorporated.

One or more of the lipids may be functionalized lipids. In some embodiments, the reactive group is one that will react with a crosslinker (or other moiety) to form crosslinks between such functionalized lipids. The reactive group may be located anywhere on the lipid that allows it to contact a crosslinker and be crosslinked to another lipid in an adjacent apposed bilayer. In some embodiments, it is in the head group of the lipid, including for example a phospholipid. An example of a reactive group is a maleimide group. Maleimide groups may be crosslinked to each other in the presence of dithiol crosslinkers such as but not limited to dithiolthrietol (DTT). An example of a functionalized lipid is 1,2-dioleoyl-sn-glycero-3-phosphoethanolamine-N-[4-(p-maleimidophenyl) butyramide, referred to herein as MPB. Another example of a functionalized lipid is 1,2-distearoyl-sn-glycero-3-phosphoethanolamine-N-[maleimide(polyethylene glycol)2000] (also referred to as maleimide-PEG 2k-PE). Another example of a functionalized lipid is dioleoyl-phosphatidylethanolamine 4-(N-maleimidomethyl)-cyclohexane-1-carboxylate (DOPE-mal).

It is to be understood that the disclosure contemplates the use of other functionalized lipids, other functionalized lipid bilayer components, other reactive groups, and other crosslinkers. In addition to the maleimide groups, other examples of reactive groups include but are not limited to other thiol reactive groups, amino groups such as primary and secondary amines, carboxyl groups, hydroxyl groups, aldehyde groups, alkyne groups, azide groups, carbonyls, haloacetyl (e.g., iodoacetyl) groups, imidoester groups, N-hydroxysuccinimide esters, sulfhydryl groups, pyridyl disulfide groups, and the like.

Functionalized and non-functionalized lipids are available from a number of commercial sources including Avanti Polar Lipids (Alabaster, Ala.).

In some embodiments, compositions further comprise one or more additional agents. Examples include, but are not limited to, polymers, proteins, carbohydrates, or other natural or artificial molecular components that serve to enhance the targeting or activity of the active agent (e.g., by promoting the binding to or phagocytosis by alveolar macrophages, or by slowing down the degradation/decomposition/clearance by macrophages in other sites of the body).

### II. Treatment Methods

Embodiments of the present disclosure provide methods of using the aforementioned drug formulations (e.g., CFZ) in the treatment of disease (e.g., respiratory or inflammatory disease or cancer). The present disclosure is not limited to particular inflammatory diseases. Exemplary diseases are described herein.

The compositions described herein find use in the treatment of a variety of acute and chronic respiratory disease. Examples include, but are not limited to, asthma, bronchiolitis, bronchiolitis obliterans, chronic obstructive pulmonary disease (COPD), bronchitis, emphysema, hypersensitivity pneumonitis, idiopathic pulmonary fibrosis, pneumoconiosis, or silicosis.

Further example of inflammatory disease include, but are not limited to acute bacterial or viral infection e.g. meningitis, sepsis, malaria or chronic inflammatory diseases such as rheumatoid osteoarthritis, psoriasis, acute respiratory disease syndrome, inflammatory bowel disease (ulcerative colitis and Crohn's disease), multiple sclerosis, etc. In some embodiments, the inflammatory disease is local inflammation (e.g., at local sites such as eyes/cornea/conjunctiva, sclera, vitreous humor etc.).

In some embodiments, the compositions described herein find use in the treatment of joint inflammation (either acute or chronic) induced due to infection of any other organs via the causative microorganisms. These conditions can also be categorized as septic arthritis or infectious arthritis or inflammatory arthritis. In some embodiments, infectious arthritis is caused by Staphylococcus aureus, Streptococcus, Streptococcus pneumonia, Neisseria gonorrhoeae, Mycobacterium tuberculosis, Borrelia burgdorferi, or Haemophilus influenza.

The compositions further find use in the treatment of arthritis. Arthritis also develops in people who have infections that do not involve the bones or joints, such as infections of the genital organs or digestive organs or ocular regions. This type of arthritis is a reaction to that infection and so is called reactive arthritis. In reactive arthritis, the joint is inflamed but not actually infected.

Additional types of inflammatory disorders that may be treated as described herein include a variety of disease states, including diseases such as hay fever, atherosclerosis, arthritis (rheumatoid, bursitis, gouty arthritis, osteoarthritis, polymyalgia rheumatic, etc.), asthma, autoimmune diseases, chronic inflammation, chronic prostatitis, glomerulonephritis, nephritis, inflammatory bowel diseases, pelvic inflammatory disease, reperfusion injury, transplant rejection, vasculitis, myocarditis, colitis, appendicitis, peptic ulcer, gastric ulcer, duodenal ulcer, peritonitis, pancreatitis, ulcerative colitis, seudomembranous colitis, acute colitis, ischemic colitis, diverticulitis, epiglottitis, achalasia, cholangitis, cholecystitits, hepatitis, Crohn's disease, enteritis, Whipple's disease, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, pneumonitits, pneumoultramicroscopic silicovolcanoconiosis, alvealitis, bronchiolitis, pharyngitis, pleurisy, sinusitis, influenza, respiratory syncytial virus infection, HIV infection, hepatitis B virus infection, hepatitis C virus infection, herpes virus infection disseminated bacteremia, Dengue fever, candidiasis, malaria, filariasis, amebiasis, hydatidcysts, burns, dermatitis, dermatomyositis, sunburn, urticaria, Warts, Wheals, vasulitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, adult respiratory distress syndrome, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thyroiditis, systemic lupus erythematosis, Goodpasture's syndrome, Behcet's syndrome, allograft rejection, graft-versus-host disease, Type I diabetes, Type II diabetes, ankylosing spondylitis, Berger's disease, Reiter's syndrome, Hodgkin's disease, ileus, hypertension, irritable boWel syndrome, myocardial infarction, sleeplessness, anxiety and stent thrombosis.

Pharmaceutical formulations include those suitable for oral, rectal, nasal, topical (including buccal and sub-lingual), vaginal or parenteral (including intramuscular, sub-cutaneous and intravenous) administration or in a form suitable for administration by inhalation, insufflation or by a transdermal patch. Transdermal patches dispense a drug at a controlled rate by presenting the drug for absorption in an efficient manner with minimal degradation of the drug. Typically, transdermal patches comprise an impermeable backing layer, a single pressure sensitive adhesive and a removable protective layer with a release liner. One of ordinary skill in the art will understand and appreciate the techniques appropriate for manufacturing a desired efficacious transdermal patch based upon the needs of the artisan.

The compounds described herein, optionally together with a conventional adjuvant, carrier, or diluent, may thus be placed into the form of pharmaceutical formulations and unit dosages thereof and in such form may be employed as solids, such as tablets or filled capsules, or liquids such as solutions, suspensions, emulsions, elixirs, gels or capsules filled with the same, all for oral use, in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compounds or principles and such unit dosage forms may contain any suitable effective amount of the active ingredient commensurate with the intended daily dosage range to be employed.

The dose when using the compounds and formulations described herein can vary within wide limits and as is customary and is known to the physician, it is to be tailored to the individual conditions in each individual case. It depends, for example, on the nature and severity of the illness to be treated, on the condition of the patient, on the compound employed or on whether an acute or chronic disease state is treated or prophylaxis is conducted or on whether further active compounds are administered in addition to the compounds. Representative doses include, but not limited to, about 0.001 mg to about 5000 mg, about 0.001 mg to about 2500 mg, about 0.001 mg to about 1000 mg, 0.001 mg to about 500 mg, 0.001 mg to about 250 mg, about 0.001 mg to 100 mg, about 0.001 mg to about 50 mg and about 0.001 mg to about 25 mg. Multiple doses may be administered during the day, especially when relatively large amounts are deemed to be needed, for example 2, 3 or 4 doses. Depending on the individual and as deemed appropriate from the patient's physician or caregiver it may be necessary to deviate upward or downward from the doses described herein.

The amount of active ingredient, or an active salt or derivative thereof, for use in treatment will vary not only with the particular salt selected but also with the route of administration, the nature of the condition being treated and the age and condition of the patient and will ultimately be at the discretion of the attendant physician or clinician. In general, one skilled in the art understands how to extrapolate in vivo data obtained in a model system, typically an animal model, to another, such as a human. In some circumstances, these extrapolations may merely be based on the weight of the animal model in comparison to another, such as a mammal, preferably a human, however, more often, these extrapolations are not simply based on weights, but rather incorporate a variety of factors. Representative factors include the type, age, weight, sex, diet and medical condition of the patient, the severity of the disease, the route of administration, pharmacological considerations such as the activity, efficacy, pharmacokinetic and toxicology profiles of the particular compound employed, whether a drug delivery system is utilized, on whether an acute or chronic disease state is being treated or prophylaxis is conducted or on whether further active compounds are administered in addition to the compounds described herein and as part of a drug combination. The dosage regimen for treating a disease condition with the compounds and/or compositions is selected in accordance with a variety factors as cited above. Thus, the actual dosage regimen employed may vary widely and therefore may deviate from a preferred dosage regimen and one skilled in the art will recognize that dosage and dosage regimen outside these typical ranges can be tested and, where appropriate, may be used in the methods described herein.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations. The daily dose can be divided, especially when relatively large amounts are administered as deemed appropriate, into several, for example 2, 3 or 4 part administrations. If appropriate, depending on individual behavior, it may be necessary to deviate upward or downward from the daily dose indicated.

The compounds can be administrated in a wide variety of oral and parenteral dosage forms. It will be obvious to those skilled in the art that the following dosage forms may comprise, as the active component, either a compound described herein or a pharmaceutically acceptable salt, solvate or hydrate of a compound described herein.

For preparing pharmaceutical compositions, the selection of a suitable pharmaceutically acceptable carrier can be either solid, liquid or a mixture of both. Solid form preparations include powders, tablets, pills, capsules, cachets, suppositories and dispersible granules. A solid carrier can be one or more substances which may also act as diluents, flavoring agents, solubilizers, lubricants, suspending agents, binders, preservatives, tablet disintegrating agents, or an encapsulating material.

Liquid form preparations include solutions, suspensions and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution and isotonic sodium chloride solution. In addition, sterile, fixed oils are conventionally employed, as a solvent or suspending medium. For this purpose any bland fixed oil may be employed including synthetic mono- or diglycerides. In addition, fatty acids such as oleic acid find use in the preparation of injectables.

The compounds according may thus be formulated for parenteral administration (e.g. by injection, for example bolus injection or continuous infusion) and may be presented in unit dose form in ampoules, pre-filled syringes, small volume infusion or in multi-dose containers with an added preservative. The pharmaceutical compositions may take such forms as suspensions, solutions, or emulsions in oily or aqueous vehicles and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Administration to the respiratory tract may also be achieved by means of an aerosol formulation in which the active ingredient is provided in a pressurized pack with a suitable propellant. If the compounds or pharmaceutical compositions comprising them are administered as aerosols, for example as nasal aerosols or by inhalation, this can be carried out, for example, using a spray, a nebulizer, a pump nebulizer, an inhalation apparatus, a metered inhaler or a dry powder inhaler. Pharmaceutical forms for administration of the compounds as an aerosol can be prepared by processes well known to the person skilled in the art. For their preparation, for example, solutions or dispersions of the compounds in water, water/alcohol mixtures or suitable saline solutions can be employed using customary additives, for example benzyl alcohol or other suitable preservatives, absorption enhancers for increasing the bioavailability, solubilizers, dispersants and others and, if appropriate, customary propellants, for example include carbon dioxide, CFCs, such as, dichlorodifluoromethane, trichlorofluoromethane, or dichlorotetrafluoroethane; and the like. The aerosol may conveniently also contain a surfactant such as lecithin. The dose of drug may be controlled by provision of a metered valve.

In formulations intended for administration to the respiratory tract, including intranasal formulations, the compound will generally have a small particle size for example of the order of 50 microns or less. Such a particle size may be obtained by means known in the art, for example by micronization. When desired, formulations adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules and powders in vials or ampoules. Also, the unit dosage form can be a capsule, tablet, cachet, or lozenge itself, or it can be the appropriate number of any of these in packaged form.

### III. Imaging

Embodiments of the present disclosure provide compositions and methods for imaging. In some embodiments, the compositions are used as imaging agents in photoacoustic tomography (PAT). Photoacoustic (PA) detection relies on intrinsic absorption at specific excitation laser-generated wavelengths resulting in ultrasonic waves detected via conventional acoustic transducers. For imaging applications, longer wavelengths are advantageous because they afford greater imaging depth, with reduced potential for phototoxicity. Accordingly, in some embodiments, provided herein is the use of drug crystals comprising clofazimine as described herein as PAT imaging or contrast agents to identify inflammation. In some embodiments, PAT is used in combination with ultrasound imaging.

In some embodiments, PAT imaging with drug crystal contrast agents is utilizing to identify inflammation in a joint (e.g., a knee joint, a finger joint, a toe joint, a hip joint, and an elbow joint). In some embodiments, the inflammation is associated with arthritis in the joint. Thus, in some embodiments, the presence of a PAT signal associated with a drug crystal in a joint is indicative of a diagnosis of arthritis or other inflammation in the joint.

### Experimental

### EXAMPLE 1

### Interplay of Lysosomal H+and Cl- on the Intracellular Disposition of Weakly Basic Drug Inclusions

### Materials and Methods

Synthesis of CFZ-HCl crystals. CFZ-HCl was synthesized as published before (Keswani et al., 2015) by adding equal volumes of 1 M NH₄Cl in H₂O to 2 mM CFZ (Sigma-Aldrich, St. Louis, Mo., Cat. No. C8895) in methanol. Subsequently, CFZ-HCl crystals were separated by centrifuging the solution at 2500×g for 20 minutes thrice, resuspending the precipitates in H₂O, freezing immediately in liquid nitrogen followed by freeze-drying in Labconco Freezone 1 benchtop freeze dry system (Labconco Corporation, Kansas City, Mo.) for 36-48 hours. The dry crystals were stored at −20° C. until further use.

Solubility Measurement Using the Flynn Approach. Freeze-dried CFZ-HCl (25 mg) samples were weighed out and separated into 5 scintillation vials, each containing 25 mg of CFZ-HCl crystals. Mili-Q water (15 mL) was added to each vial ensuring that CFZ-HCl crystals were in great excess. Moreover, an appropriate amount of 0.1 M NaOH was added to each vial to set the initial equilibration pH measurements as follows: Sample vials 1-5 initially contained 0, 40, 80, 120, and 200 μl of 0.1 M NaOH solution, respectively, and then after 24 hour equilibration period, 10 μl of 0.1 M NaOH was added each day for a period of five days resulting in pH range of 4.5 to 8. 9. The set of sample containing vials was placed on a magnetic stirrer plate in a 25° C. water bath. Each sample was allowed to equilibrate for at least 24 hours, after which 500 μl of sample was taken out and filtered through Spin-X centrifuge tube filters (0.45 um cellulose acetate, 2 ml polypropylene tubes, non-sterile, Costar®, Cat # 8163) for 4 min @ 10000 rpm. The pH of the filtered sample was determine by Denver Instrument UltraBasic pH meter (Denver Instrument, Bohemia, N.Y.), after which the sample was subjected to HPLC analysis (Waters Alliance, Separations Module 2695) to determine the total solubility of the drug at the measured pH. The mobile phase was chosen to be 80:20 (methanol:water +0.1% trifluoroacetic acid) with 1 ml/min flow rate. The stationary phase (column) was chosen to be C18 (unbonded silica particles) column (Atlantis® T3, 5 μm, 100 Å) and the HPLC was equipped with a UV detector (Waters, Photodiode Array Detector 2996) @ 285 nm detection for CFZ. The retention time for CFZ was determined to be at 4.75 min. For each sample, solubility measurement was performed in triplicates, and the average was used to construct the pH solubility profile. The standard curve was generated using CFZ-HCl crystals, dissolved in the mobile phase at known concentrations (1, 5, 10, 20, 30, 40, and 50 μM).

Determination of CFZ Solubility Parameters from the Flynn Approach. For weak electrolytes such as weakly basic drugs, the protonation state of the drug (B) is dictated by the relationship between the drug's association constant (Ka) and the acidity of its environment (H3O). This is represented by the following equilibrium expression.

B+H₃O→BH⁺+H₂O (1)

Where BH⁺ is the protonated form of the drug as a result of the interaction of the free base form of the drug with hydronium ion, H₂O is the byproduct of the reaction and remains constant; hence, it is incorporated in the association constant. Therefore, equation 1 can be re-written using the new apparent association constant (Ka′).

B+H₃O →BH⁺ (2)

where Ka′=Ka*H₂O.

Thus, the mass law equilibrium equation can be written as the following assuming an ideal solution where the activity of a given species equals the concentration of the species:

Ka′=[BH⁺]/[B][H₃O] (3)

Where [B] is the concentration of the neutral form of the drug, [BH⁺] the concentration of the protonated form of the drug, and [H₃O] the concentration of the hydronium ion.

By writing the logarithmic form of equation 3, the following relationship, which is also famously known as the Henderson-Hasselbalch equation, is obtained:

pH−pKa=log([B]/[BH⁺]) (4)

The equation can be re-written as the following to express the concentration of both the neutral and ionized forms of the drug.

[B]=[BH⁺]*10^{p}^{^{H-P}}^{_{K}}^{a} (5)

[BH⁺]=[B]*10^{P}^{_{K}}^{a-pH} (6)

According to thermodynamic laws of mass action, the total amount of the drug must comprise of both the neutral as well as the protonated forms of the drug at any pH of its environment. Thus, the total solubility (ST) of the drug can be written as:

S^{T}=[B]+[BH⁺] (7)

However, depending on solution pH, the total drug solubility equation is slightly modified to account for the distinction between primary species in the solid phase versus in solution phase. For pH<pHmax, the ionized form of the drug is in the solid phase and therefore, remains constant while the neutral form of the drug varies with respect to pH. Therefore, by substituting equation 5 into equation 7 the total solubility for pH<pHmax can be re-written as:

S^{T}=[BH⁺]_{S}*(1+10^{pH-P}^{_{K}}^{a}) (8)

Where the solid phase is denoted by the subscript “s”.

To the contrary, for pH>pHmax, the neutral form of the drug is in the solid phase, and hence remains constant while the ionized form of the drug varies with respect to pH. Therefore, by substituting equation 6 into equation 7 the total solubility for pH<pHmax can be re-written as:

S_{T}=[B]_{S}*(1+10^{p}^{_{K}}^{a-pH)} (9)

Moreover, at pH=pHmax, both the ionized and the neutral forms of the drug are present in the solid phase. Thus, equation 7 can be re-written as:

S_{T}=[B]_{S}+[BH⁻]_{S} (10)

Thus, in order to fit the experimental solubility-pH data curve of CFZ-HC, it was determined which one of the aforementioned solubility-pH relationships (equation 8 vs. equation 9) to use. Because the precipitation of the free base as a function of pH increment was observed, it was deduced that the total solubility-pH relationship with the free base form of the drug species in the solid phase could fit the experimental solubility-pH data. Thus, by substituting CFZ, equation 7 is re-written as follows:

S_{T}=[CFZ]_{S}+[CFZH⁺] (11)

Where the solid phase, which in this case is the free form of CFZ and is denoted by the subscript “s”, while the ionized form of the drug, [CFZH⁺], is in the solution phase and similar to equation 6, it is dictated by pKa and pH as follows:

[CFZH⁺]=[CFZ]_{S}*10^{p}^{_{K}}^{a-pH} (12)

Thus, by plugging equation 12 into equation 11, one obtains

S==[CFZ]^{S}*(1+10^{p}^{_{K}}^{a-pH}) (13)

Furthermore, by using different combinations of any two solubility-pH data points from the experimental measurements, the pKa and the intrinsic free base solubility values were solved simultaneously using equation 13. However, out of the different combinations, it was the combination of the first and last solubility-pH data points which gave the least calculation error, and hence what was used in follow-up computations. Thus, using the calculated intrinsic free base solubility and pKa, the experimentally obtained solubility-pH curve of CFZ-HCl was obtained by calculating the total solubility for the given range of pH 4.5 to 8.9. Furthermore, using the other total solubility versus pH curve generating equation, which assumes the salt form of the drug to be in the solid phase, the total solubility-pH dataset was repeated so that the pH where the two total solubility-pH curves intersect can be deduced as the pHmax (Kramer and Flynn, J Pharm Sci 61:1896-1904 1972).

S_{T}=[CFZ]+[CFZH⁺]_{S} (14)

Similar to equation 8, equation 14 can be further elaborated as:

S_{T}=[CFZH⁺]_{S}*(1+1^{pH-p}^{_{K}}^{a}) (15)

However, [CFZH⁺]_{S} is not known. Thus, a mathematical proof approach was used to determine [CFZH⁺]_{S} as detailed below:

First, [CFZH⁺] was calcuatued using the total solubility (S_{T}) at each pH value and the constant intrinsic free base solubility, [CFZ]_{S}, using the following equation, which was obtained by rearranging the terms in equation 14:

[CFZH⁺]=S_{T}−[CFZ]_{S} (16)

Then, a value, say “y”, from the list of the computed [CFZH⁺] values was set equal [CFZH₊]_{S}. By substituting “y’ in place of [CFZH₊]_{S}, total solubility at the different pH values was calculated using the solubility-pH equation where the intrinsic salt is the primary solid species (equation 15). This total solubility vs. pH dataset (obtained using equation 15) was compared to that of total solubility vs. pH dataset (obtained using equation 13). Then, it was determined if there was the same total solubility value at a given pH in both datasets, which represents the intersection point of the two solubility-pH curves mentioned before, where both the intrinsic free base and salt coexist in the solid phase. This can be represented by the following equation:

S_{T}′=[CFZ]_{S}+[CFZH₊]_{S} (17)

Where S_{T}′ is the total solubility value at the intersection of the two solubility-pH curves and both forms of the drug, CFZ and CFZH⁺, are in the solid phase denoted by the subscript s.

Moreover, to prove if the earlier assumption, “y” is equal to [CFZH₊]_{S}, was valid, the total solubility was calculated by plugging in “y” in place of [CFZH₊]_{S} (equation 17) and checked if it was equal to S_{T}′. Furthermore, by definition, the pH where both the intrinsic free base and the salt form of the drug are in the solid phase is known as pHmax. Thus, the pH associated with S_{T}' was deduced to be the pHmax.

Epifluorescence Microscopy of in vitro RAW264.7 cell culture. RAW264.7 cells (ATCC, Manassas, Va., ATCC Number: TIB-71TM) at a very high seeding density of 200,000 cells/well, grown in a 8-chamber multiwell plate (Lab-Tek® II, Nunc, Rochester, N.Y.) in 500 μl/well of Dulbecco's Modified Eagles Medium (DMEM) +10% Fetal Bovine Serum (FBS) +1% Penicillin/Streptomycin (P/S) (growth media), were pre-incubated with CFZ at 10-20 μM CFZ using a stock at a concentration of 2 mM in DMSO for 24-72 hours. Visualization of all samples (cells or crystals) was done on a Nikon Eclipse Ti (Nikon Instruments, Melville, N.Y.). The fluorescence filters (excitation/emission) used were optimized for 4,6-diamidino-2-phenylindole dihydrochloride (DAPI) (350/405 nm, exposure-550 ms), fluorescein isothiocyanate (FITC) (490/510 nm, exposure—100-500 ms), Texas Red (590/610 nm, exposure<500 ms), and Cy5 (640/670 nm, exposure—500 ms). Brightfield color photographs were acquired using a Nikon DS-Fi2 camera, whereas fluorescence photographs were acquired using a Photometrics CoolSNAPTM MYO (Photometrics, Tucson, Ariz.) camera.

Spectral Confocal Microscopy. For the preparation of slides, a 20 μl drop of CFZ-HCl drug crystals suspended in Phosphate Buffer Solution (PBS) was placed on a glass slide and a cover slip was applied onto the sample prior to imaging. Spectral confocal microscopy was performed on a Leica Inverted SPSX confocal microscope system with two-photon FLIM (Leica Microsystems, Buffalo Grove, Ill.) using excitation wavelengths (470-670 nm). Image analysis and quantification was performed on Leica LAS AF. Several regions of interest of individual crystals were used to obtain fluorescence data which were imported into MS-Excel for further analysis. All fluorescence yields were normalized to the maximum fluorescence yield measured across the tested spectral range and background subtracted using data obtained from a blank slide.

Computational Simulation of a Macrophage lysosome. A Lysosomal ion regulation model which incorporates lysosomal proteins such as V-ATPase, CLC7, and membrane proton permeability was adopted (Ishida et al., J Gen Physio1141:705-720 2013) and further elaborated to understand the relationship between the regulation of lysosomal physiology, ion homeostasis, and intracellular CFZ accumulation.

Ion Transportation

Proton Flux. V-ATPase is an electrogenic proton pump which inserts protons from the cytoplasm into the lysosome against an electrochemical gradient upon ATP hydrolysis (Grabe et al., Biophys J78:2798-2813 2000; Grabe and Oster, J Gen Physiol 117:329-344 2001). The rate at which a proton molecule is inserted per second (JHVATP) was obtained from experimental studies detailed in (Grabe et al., Biophys J 78:2798-2813 2000) as a function of transmembrane pH gradient (A pH) in units of pH unit and membrane potential difference which is also interchangeably known as membrane potential (ΔΨ) in units of mV. This rate is multiplied by the total amount of active V-ATPase molecules per lysosome (NVATP) to obtain the total amount of proton molecules inserted into the lysosome in units of molecules per second, as follows:

Hₚᵤₘₚ=N_{VATP}*J_{HVATP} (ΔpH (ΔΨ) (18)

Where lysosomal membrane potential (ΔΨ) is dictated by the total net change in lysosomal ion content and is represented by the following relationship.

$$ΔΨ = \frac{{FV}_{L}}{{C}^{′} S} * [ ( {\sum }_{i} {{Z}_{i} [ cations ]}_{i} + {\sum }_{i} {{Z}_{i} [ anions ]}_{i} ) - B ] \qquad ( 19 )$$

Where V_{L} is the lysosomal volume in units of L; F Faraday's constant which equals 96485 Coulomb/mol and is used to convert the lysosomal ion content in units of moles to units of Coulomb; C′ the specific membrane capacitance per unit area of a biological membrane, which has been experimentally approximated to be 1 μF/cm², and is multiplied by the lysosomal surface area S represented in units of cm², to obtain the total lysosomal membrane capacitance; Z₁ valence for ion i; [cation] the concentration of cation i at a given time tin units of Molar; [anion] the concentration of anion i at a given time t in units of Molar; B the Donnan particles, in units of Molar, are impermeable lysosomal contents defined by initial lysosomal ion concentrations and net change in the intrinsic surface potentials, equation 20. In the model and simulations communicated in this report, the initial lysosomal ions consist of proton, potassium, sodium and chloride with their respective charges (+or −1) as coefficient of their respective concentrations, represented by the square brackets, equation 20.

$$B = {[ {H}^{+} ]}_{L , initial} + {[ {K}^{+} ]}_{L , initial} + {[ {Na}^{+} ]}_{L , initial} - {[ {Cl}^{-} ]}_{L , initial} - \frac{CS}{{FV}_{L}} * [ ( {Ψ}_{in} - {Ψ}_{out} ) + {Ψ}_{initial} ) \qquad ( 20 )$$

Where Ψᵢₙ and Ψₒᵤₜ are the intrinsic inner and outer surface potentials, respectively, in units of mV, which contribute to the change in ion concentration at membrane surface, Tinitial is the initial lysosomal membrane potential which is set to zero mV in order to maintain initial lysosomal membrane electroneutrality.

Chloride Flux. Following V-ATPase mediated proton influx into the lysosome; lysosomal membrane potential increases and therefore arrests further proton influx. Thus, to lower the membrane potential for the continuation of V-ATPase proton-pumping activity which is essential to lower lysosomal pH to physiological pH, lysosomal cation removal or the insertion of anion into the lysosome is required. Accordingly, CLC7 is considered as the primary membrane potential dissipating protein which transports two chloride ions from the cytoplasm to the lysosome for every proton it transports from the lysosome to the cytoplasm (Graves et al., Nature 453:788-792 2008). The rate (JCl, HClC7) at which the ion transportations occur was empirically derived from a current-voltage experimental data communicated in Ishida et al (J Gen Physiol 141:705-720) as a function of chemical (ΔpH, ΔCl) and electric potential gradients, (ΔΨ), equations 21 and 22. This rate was multiplied by the total number of CLC7 molecules per lysosome (N_{ClC}) to obtain the total amount of proton and chloride molecules transported across the lysosomal membrane through CLC7, as follows.

H_{ClC7}=N_{ClC7}*J_{Cl,H}_{_{ClC7}}(ΔpH, ΔCl, ΔΨ) (21)

Cl_{ClC7}=2*N_{ClC7}*I_{CLH}_{_{ClC7}}(ΔpH, ΔCl, ΔΩ) (22)

Where ΔCl is the chloride gradient comprised of the luminal chloride (ClL) and the cytoplasmic chloride (ClC). Moreover, the coefficient 2 in equation 22 defines the 2:1 stoichiometric relationship between the chloride and proton ions transported by CLC7.

Proton Leak. Furthermore, in addition to CLC7, a passive diffusion of protons across the lysosomal membrane can also contribute to the dissipation of membrane potential in order to facilitate the proton pumping activity of V-ATPase. The passive proton transportation dictated by electrochemical gradient is captured by equation 23, which is derived from the Goldman Hodgkin Katz (GHK) ion flux equation (Weiss, Cellular Biophysics: Transport. MIT Press 1996), which is a commonly used equation to describe the passive diffusion of a given ion across a biological membrane, assuming a linear potential gradient across the lipid membrane.

$${H}_{leak} = ( {SP}_{H} + YZ * \frac{{10}^{- {pH}_{L}} - ( {10}^{- {pH}_{C}} * {e}^{- ZY} )}{1 - {e}^{- ZY}} ) * {N}_{ev} \qquad ( 23 )$$

Where S is the total lysosomal surface area in units of cm2 used to obtain the total amount of proton which passively diffuses across the lysosomal membrane; P_{H}+ the lysosomal membrane proton permeability in units of cm/s; Z the valence of the ion, which in this case is +1 for proton; pH_{L} the luminal pH used to calculate the total free lysosomal proton based on the logarithmic pH and free proton relationship (pH_{L}=−log[H+]); pH_{c} the cytoplasmic pH used to calculate the total free cytoplasmic proton based on the logarithmic pH and free proton relationship (pH_{C}=−log[H⁺]); Nav Avogadro's number used to convert the amount of transported protons in unit of moles to molecules; Y used to convert proton transportation in unit of charge per second to moles per second and is defined as, Y=ΔΨF/RT, where R is universal gas constant, F Faraday's constant, and T absolute temperature. Moreover, for cells at room temperature (25° C.), RT/F equals 25.69 mV (Hille, Ionic Channels of Excitable Membranes. 2nd Edition. Sinauer Associates, Inc , Sunderland, Mass. 1992), and hence is used for normalizing the lysosomal membrane potential communicated in this report.

Moreover, to incorporate the lysosomal accumulation of CFZ-HCl as a function of lysosomal proton and chloride ions into the model, the rate of overall drug accumulation obtained from experimental findings (Min et al., 2015) was used to define the rates of both lysosomal proton (H_{sequestered}) and chloride (Cl_{sequestered}) sequestration by CFZ, in units of molecules per second. These rates were assumed to be equal to one other due to the proposed equal contribution of both proton and chloride ions in the formation of CFZ-HCl:

H_{sequestered}=Cl_{sequestered} (24)

Surface Concentration. In order to account for the effects of intrinsic external (Ψ_{i,out}) and internal (Ψ_{i,in}) leaflet potentials of the lysosomal membrane on cytoplasmic and lysosomal ion i concentrations, respectively, the individual cytoplasmic and lysosomal ion concentrations are computed using the following relationships, equations 25 and 26, derived from the GHK equation for a single ion concentration gradient, by setting net current flow equal to zero.

$$\begin{matrix}{ΔΨ}_{i , in} = \frac{- ZRT}{F} ln \frac{{C}_{i , in}}{{C}_{i , L}} & 25 \\ {ΔΨ}_{i , out} = \frac{- ZRT}{F} ln \frac{{C}_{i , out}}{{C}_{i , C}} & 26\end{matrix}$$

Where C_{i,in} is the internal concentration of a given ion i at the membrane surface facing the lysosomal compartment, C_{i,L} the concentration of the ion i inside the lysosome, C_{i,out} the external concentration of the ion i at the membrane surface facing the cytoplasmic compartment, C_{i,C} the concentration of the ion i inside the cytoplasm. All units are molar.

Governing Equations. The previously defined equations (equations 18-26) which describe ion transport across the lysosomal membrane were used in the following time-dependent ordinary differential equations (equations 27-29) to further define the ion movements as a function of time, in units of molecules per second.

$$\begin{matrix}\frac{{dH}^{+}}{dt} = {H}_{pump} - {H}_{C lC 7} - {H}_{leak} - {H}_{sequestered} & 27 \\ \frac{{d Cl}^{-}}{dt} = 2 * {N}_{C lC } * {I}_{Cl , {H}_{C lC 7}} ( Δ pH , Δ Cl , ΔΨ ) - {Cl}_{sequestered} & 28\end{matrix}$$

Moreover, extending equation 27, one can write the change in lysosomal pH with respect to time following the relationship between the lysosomal lumen buffering capacity (β) of the Donnan particles, which sequester non-free lysosomal protons, and free lysosomal protons which give rise to lysosomal pH as follows:

$$\frac{dpH}{dt} = \frac{( - {H}_{pump} + {H}_{C lC 7} + {H}_{leak} + {H}_{sequestered} )}{V * {N}_{av} * β} \qquad 29$$

Where V is the lysosomal volume and is multiplied by Nav to convert the unit of molecules per second (as in the case of equation 27) to molar per second with the inverse of β in units of pH unit per molar.

Model Parameterization

The model consists of 23 parameters: four were adjustable and the remaining 19 were fixed. Fixed parameters are those with values obtained from the literature associated with physiological lysosomal ion homeostasis. Therefore, these parametric values are interchangeably referred hereon as “baseline input values” or “physiological baseline input values” and are described in Table 3. The adjustable parameters are those varied from their respective baseline input values in order to investigate their individual as well as combined effects on the physiological lysosomal pH, Cl and membrane potential readout values, as to be discussed in the following subsections. These parameters include the number of active V-ATPase and CLC7 molecules per lysosome, as well as cytoplasmic chloride concentration. In addition, the rate of proton and chloride sequestration by CFZ (1.16×10⁻²¹ to 1.16×10⁻²⁰, obtained from wet lab experimental findings (Min et al., Adv Sci (Weinh) 2 2015) is also considered an adjustable parameter as it is something foreign to the lysosome, and hence newly introduced to the model to study the phenomenon of a weakly basic drug accumulation and stabilization within the lysosome.

TABLE 3
Table 1: Model Parameters

| | | Baseline Input | | |
|---|---|---|---|---|
| Symbol | Description | Value | Range of Input Value | Units |
| pH_{C} | Cytosolic pH | 7.2 | Fixed | pH unit |
| pH_{L} | Luminal pH | 7.4 | Fixed | pH unit |
| [Cl⁻]_{C} | Cytosolic chloride | 10 | 1 × 10⁻⁵-10 | mM |
| | concentration | | | |
| [Cl⁻]_{L} | Luminal chloride | 110 | Fixed | mM |
| | concentration | | | |
| [Na⁺]_{L} | Luminal sodium | 145 | Fixed | mM |
| | concentration | | | |
| [K⁺]_{L} | Luminal potassium | 5 | Fixed | mM |
| | concentration | | | |
| [H⁺]_{L} | Luminal proton | 0 | Fixed | mM |
| | concentration | | | |
| P_{H}⁺ | Membrane proton | 6 × 10⁻⁵ | 6 × 10⁻⁵ | cm/s |
| | permeability | | | |
| V | Lysosomal volume | 1.65 × 10⁻¹⁶ | Fixed | L |
| S | Lysosomal surface | 1.45 × 10⁻⁸ | Fixed | cm² |
| | area | | | |
| C′ | Specific bilayer | 1 | Fixed | μFarad/cm² |
| | capacitance | | | |
| β | Buffering capacity | 40 | Fixed | mM/pH unit |
| N_{VATP} | V-ATPase number | 300 | 1 × 10⁻⁴ -300 | |
| N_{ClC7} | CLC7 number | 5000 | 1 × 10⁻⁴ -5000 | |
| Ψₒᵤₜ* | Outer surface | −50 | Fixed | mV |
| | potential | | | |
| Ψᵢₙ* | Inner surface | 0 | Fixed | mV |
| | potential | | | |
| CLC_Cl | CLC7 Cl⁻ | 2 | Fixed | |
| | stoichiometry | | | |
| CLC_H | CLC7 H⁺ | 1 | Fixed | |
| | stoichiometry | | | |
| R | Gas constant | 8.314 | Fixed | J · K⁻¹ · mol⁻¹ |
| T | Absolute | 0 | Fixed | Kelvin |
| | temperature | | | |
| F | Faraday's constant | 96485 | Fixed | J/volt |
| Nₐᵥ | Avogadro's number | 6.02 × 10²³ | Fixed | molecules/mol |
| a | Rate of proton and | 0 | 1.16 × 10⁻²³ to 1.16 × 10⁻²⁰ | Moles/day |
| | chloride | | | |
| | sequestration by | | | |
| | CFZ | | | |

Baseline input values are literature values (Heuser et al., 1993; Van Dyke, 1993; Gambale et al., 1994; Sonawane et al., 2002; Alberts, 2008) representing physiological lysosomes and are in agreement with previously published model (Grabe and Oster, 2001; Ishida et al., 2013).
*estimated intrinsic surface potentials for inner (Ψᵢₙ) and outer (Ψₒᵤₜ) leaflets of the lysosomal membrane accounted for when modeling membrane transporter mediated dynamic lysomal and cytoplasmic ion concentration at the membrane surface (Grabe and Oster, 2001).

Simulating lysosomal CFZ-HCl accumulation. To simulate the dose-dependent lysosomal CFZ-HCl accumulation, the rates of proton and chloride sequestration by CFZ were adjusted to either 0.01 or 0.1 picomoles/cell/day, which equal to 1.2×10⁻²¹ moles/lysosome/day and 1.2×10⁻²⁰ moles/lysosome/day, respectively, as calculated by assuming there are approximately 100 lysosomes in a cell. Interchangeably from here onwards, these rates; 0.01 and 0.1 picomoles/cell/day, are referred to as 1-fold (1×) and 10-fold (10×) CFZ-HCl accumulation, respectively.

Simulating the inhibition of lysosomal proteins and cytoplasmic chloride. In order to study the role of V-ATPase, CLC7, and cytoplasmic chloride concentration on the lysosomal accumulation of CFZ-HCl, the total number of active V-ATPase and CLC7 molecules per lysosome, as well as the cytoplasmic chloride concentration were varied, while fixing the values of other lysosomal parameters at their baseline physiological input values in the presence and absence of lysosomal CFZ-HCl accumulation. More specifically, to simulate the simultaneous inhibition of V-ATPase and CLC7, the total number of CLC7 molecules per lysosome was varied from 0 to 5000 in geometric interval of 2.09 while one simulation at a time the total number of V-ATPase molecules per lysosome was manually varied from 0 to 300 in arbitrary intervals. Moreover, to simulate the simultaneous inhibition of V-ATPase and cytoplasmic chloride, cytoplasmic chloride concentration was varied from 0 to 10 mM in arithmetic intervals of 6.67×10-4 while one simulation at a time the total number of V-ATPase molecules per lysosome was manually varied from 0 to 300 in arbitrary intervals.

Using the aforementioned ranges of the adjustable lysosomal parameters, the corresponding inhibition range of 0 to 100% was calculated; where 0% represents no change from respective physiological baseline input value whereas 100% represents maximum change from respective physiological baseline input value. The inhibition range was calculated as follows by comparing its corresponding input value from the aforementioned given range (Adjusted Input Value) with its respective physiological input value (Baseline Input Value).

$$\% Inhibition = \frac{Baseline Input Value - Adjusted Input Value}{Baseline Input Value} * 100 \qquad 30$$

Calculating the effect of CFZ-HCl accumulation on lysosomal ion homeostasis. The set of differential equations detailed earlier (equations 27-29) was solved by numerical integration in Berkeley Madonna (BM) using Rosenbrock stiff solver as numerical integrator. Upon performing parametric simulations in the absence and presence of CFZ-HCl accumulating at either 1× or 10×, along with different ranges of lysosomal parameters such as V-ATPase, CLC7, and cytoplasmic chloride (as elaborated earlier), final lysosomal pH, chloride and membrane potential were chosen as readout values. The readout values in the absence of CFZ-HCl were subtracted from the ones in the presence of CFZ-HCl to determine the effect of lysosomal CFZ-HCl accumulation on lysosomal ion homeostasis represented by the changes in lysosomal pH, Cl, and membrane potential.

Calculating the effects of V-ATPase, CLC7, and cytoplasmic chloride on the physiological lysosomal accumulation of CFZ-HCl. Time-plot simulations were performed to obtain final lysosomal pH, chloride, and membrane potential values (specifically chosen because they are direct indicators of lysosomal ion homeostasis and physiology) while adjusting all of the lysosomal parameters to their respective baseline values in the absence of CFZ-HCl accumulation. This allowed us to obtain physiological lysosomal pH of 4.53, chloride concentration of 224.6 mM, and membrane potential of 0.79 mV, which from here onwards thes values are referred to as “physiological baseline readout values” as they are associated with no changes to physiological baseline lysosomal parameters and contents.

Moreover, in the presence of CFZ-HCl accumulating at 1× and 10×, final lysosomal pH, Cl and membrane potential values were chosen as readout values while performing parametric simulation runs in the presence of simultaneous V-ATPase-CLC7 and V-ATPase-cytoplasmic chloride inhibitions. These values were then subtracted from the physiological baseline readout values to calculate the effects of the simultaneous lysosomal parameter inhibitions on dose-dependent lysosomal CFZ-HCl accumulation.

Steady state and mass balance check. For all of the aforementioned simulations, the final readout values were confirmed that they were steady state values by performing the simulation for >24 hrs. Moreover, mass balance was maintained in all of the simulations in the presence as well as absence of CFZ-HCl accumulation as long as physiological pH gradient of up to 4.6 pH units, associated with the hydrolysis of 3 ATP molecules, was maintained.

Generating 3D Plots. All of the readout values in the absence and presence of CFZ-HCl were obtained as a function of a lysosomal parameter that was varied using the parametric plot feature in BM while manually adjusting one simulation at a time an additional lysosomal parameter to a fixed value from a chosen range of values. Thus, multiple two-dimensional datasets, corresponding to the total number of values chosen for the additional lysosomal parameter, were saved as Notepad files and exported to Microsoft Excel. The 2D datasets were further categorized in an Excel spreadsheet depending on the additional lysosomal parameter being varied. For example, when simultaneously inhibiting V-ATPase and CLC7 in the presence or absence of lysosomal CFZ-HCl accumulation, 2D lysosomal readout values were obtained as a function of the total number of CLC7 molecules per lysosome for each adjusted input value of the total number of V-ATPase molecules per lysosome ranging from 0 to 300. Thus, each set of 2D lysosomal readout values as a function of the total number of CLC7 molecules per lysosome is categorized in the Excel spreadsheet under a row clearly identified with the associated value of the total number of V-ATPase molecules per lysosome.

Furthermore, a matrix was generated for each individual lysosomal readout value. For example, in the case of simultaneous V-ATPase and CLC7 inhibition in the presence of CFZ-HCl accumulation at 1×, the total number of variables for the total number of V-ATPase and CLC7 molecules per lysosome were 7 and 16, respectively. Thus, the matrix was 16 by 7, where the lists of the total number of CLC7 and V-ATPase molecules per lysosome were chosen as the row and column of the matrix, respectively, and the lysosomal readout values corresponding to the different combinations of the total number of CLC7 and V-ATPase molecules per lysosome were contained within the matrix itself. The matrix containing the calculated results was then exported to SigmaPlot® to generate a three-dimensional plot. In the 3D plot, the Z axis corresponds to the calculated result, while the X and Y axes correspond to the varied lysosomal parameters.

Cell Viability. Viability of RAW264.7 cells were performed using Cell Proliferation Kit II (XTT) (Roche Applied Science, Mannheim, Germany, Cat. No. 11465015001) using the manufacturer's instructions. RAW264.7 cells at a very high seeding density of 50,000 cells/well, grown in a 96 well tissue culture plate in 280 μl/well of Dulbecco's Modified Eagles Medium (DMEM) +10% Fetal Bovine Serum (FBS) +1% Penicillin/Streptomycin (P/S) (growth media), were pre-incubated with varying concentrations of BafA1 (Sigma-Aldrich, St. Louis, Mo., Cat. No. B1793) (0-10 nM) and NPPB (Sigma-Aldrich, St. Louis, Mo., Cat. No. N4779) (0-200 μM) for 4 hours before adding CFZ dissolved in DMSO such that final concentrations of CFZ were in the range of 0-10 μM. Post 24 hours exposure to CFZ, the cells were incubated with the yellow XTT solution (final concentration 0.3 mg/ml) for 4 h. After this incubation period, orange formazan solution was formed, which was spectrophotometrically quantified using a Synergy 2 plate reader (BioTek, Winooski, Vt.) at 500 nm and 700 nm (for background). An increase in number of living cells directly correlates to the amount of orange formazan formed, as monitored by the solution absorbance at 500 nm (Abs500). The following formula was used to determine viability of drug and inhibitor treated cells using untreated cells (blank) as a control—Cell Viability=(Abs500, treated—Abs700,treated)/(Abs500, blank—Abs700,blank).

Drug Uptake. Measurement of CFZ uptake in RAW264.7 cells was performed using a modified absorbance spectroscopy method using 9 M H2SO4 (pH<<0.1) to digest the entire cell population and extract CFZ from the cells. Cells at a very high seeding density of 50,000 cells/well, grown in a 96 well tissue culture plate in 280 μl/well of DMEM +10% FBS +1% P/S (growth media), were pre-incubated with varying concentrations of BafA1 (0-4 nM) and NPPB (0-200 μM) for 4 hours before adding CFZ dissolved in DMSO such that final concentrations of CFZ were in the range of 0-8 μM. Post 4 and 24 hours exposure to CFZ, growth media was aspirated out and the cells were washed with PBS twice before adding 100 μl/well of 9 M H2SO4 and incubated at room temperature for 30 minutes before spectrophotometric quantification using a Synergy 2 plate reader (BioTek, Winooski, Vt.) at 540 nm (Abs540) and 750 nm (Abs750) (for background). Standards were prepared on the same plate by adding 100 μl/well pre-determined standards of CFZ. Total uptake was reported as total picomoles of intracellular drug as measured and calculated using the standard curve.

Cell Viability Measurement: Comparing Wet Lab Experimental Study with Simulation Study. In order to compare wet lab experimental cell viability determination with that of model simulation, the rate at which drug accumulation occurred in the experimental study was calculated. This was done assuming the doubling time of 50,000 cells/well to be approximately 11 hours (Sakagami et al., Anticancer Res 29:343-347 2009), and calculating the amount of drug in individual cells in units of picomole per cell for each extracellular CFZ concentration in the absence of any inhibitor mentioned earlier. Then, the extracellular CFZ concentration that's closely associated with the rate of drug accumulation at either 0.01 picomole/cell/day or 0.1 picomole/cell/day, which are the rates used in the modeling and simulation study as mentioned earlier, was chosen as the comparable experimental dose to the modeling and simulation dose. Then, cell viability was calculated using the following equation:

$$\% Cell Viability = 100 \% - ( \frac{\begin{matrix}Lysosomal pH - \\ Baseline lysosomal pH\end{matrix}}{Baseline lysosomal pH} ) * 100 \% \qquad 31$$

Where the baseline lysosomal pH, is as mentioned before, one of the lysosomal readout values, specifically used in this case as a direct indicator of cell viability, and is associated with baseline physiological lysosomal parameters including 300 V-ATPase and 5000 CLC7 molecules per lysosome. Whereas, the lysosomal pH corresponds to all of the readout lysosomal pH values in the presence as well as absence of V-ATPase and CLC7 inhibitors.

Drug Uptake Measurement: Comparing Wet Lab Experimental Study with Simulation Study. First, simulated V-ATPase and CLC7 inhibition ranges associated with viable lysosomes which can accommodate CFZ-HCl accumulation at 1× or 10× were determined. For these ranges, the lysosomal pH of CFZ-HCl free lysosome was subtracted from that of CFZ-HCl containing lysosome in order to quantify the physiological difference between CFZ-HCl containing and CFZ-HCl free lysosomes. The lysosomal pH difference of the CFZ-HCl containing lysosome and CFZ-HCl free lysosome under baseline physiological V-ATPase (300 V-ATPase molecules per lysosome) and CLC7 (5000 CLC7 molecules per lysosome), symbolized by ApHb, was used as the baseline pH difference associated with maximum drug uptake. Moreover, as the lysosomal pH difference (ApH) between CFZ-HCl containing and CFZ-HCl free lysosomes increases, (ΔpH>ΔpHb), it indicates deviation from maximum drug uptake. Thus, using the inverse relationship of ΔpH to % drug uptake, % drug uptake of CFZ in the presence of the defined V-ATPase and CLC7 inhibition ranges associated with viable lysosome can be calculated as follows:

$$\% Drug Uptake = \frac{Δ {pH}_{b}}{ΔpH} * 100 \% \qquad 32$$

### Results

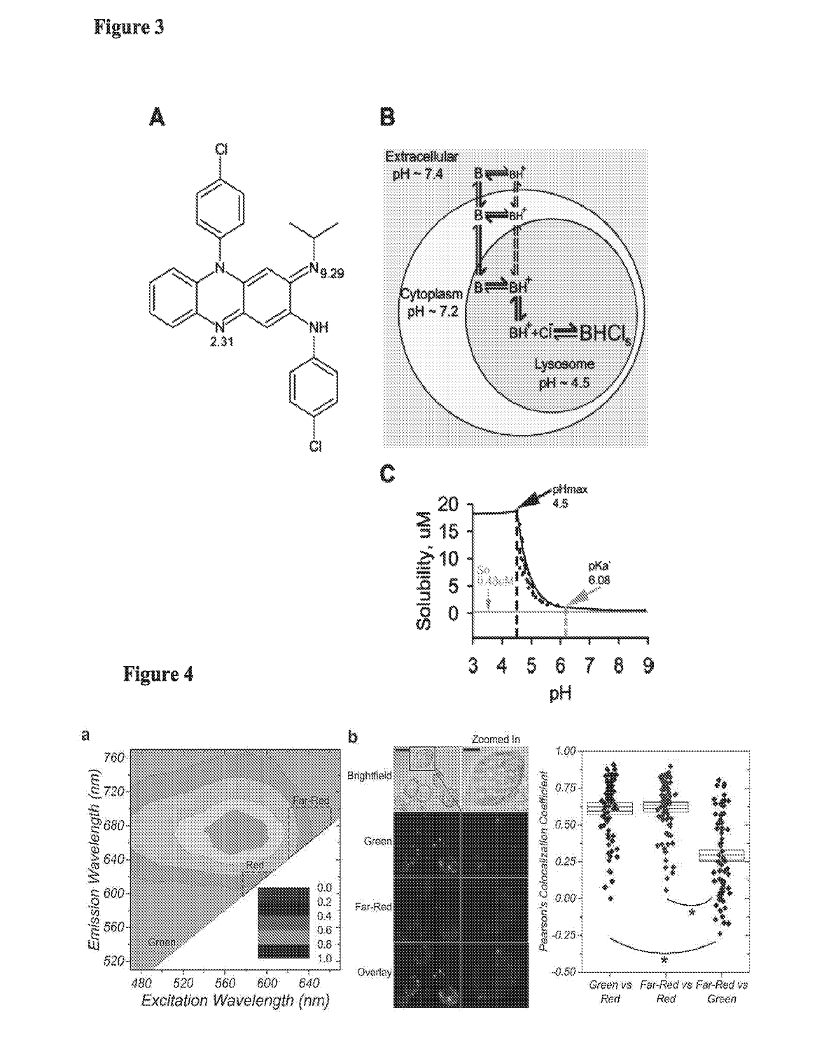

Estimating and measuring the weakly basic chemical characteristics of CFZ (Ksp, pKa). CFZ is identified as a weakly basic, lipophilic drug and has four amine groups. While its biochemistry has been described previously, experimental evidence of its chemical nature is sparse. Using ChemAxon®, it was estimated that two amines can be protonated within the pH range of 0-9 with the pKa,1=2.3 and pKa,2=9.29 (FIG. 3A). This indicated that within a cell, the majority of the drug would be present and bioaccumulate as monoprotonated species within the cell (FIG. 3B). To confirm this value using an experimental approach, a pH-dependent solubility study was performed using Gordon-Flynn (Kramer and Flynn, 1972, supra) approach, which allowed one to accurately measure the total solubility of CFZ-HCl as a function of pH. From the experimental measurements, the solubility properties of CFZ including its apparent pKa,2 (pKa=6.08±2.43×10-3; 95% Confidence Interval=6.07, 6.09) and intrinsic free base solubility (S0=0.48±4.05×10-6 μM; 95% CI=0.48, 0.48) were calculated (FIG. 3C). Moreover, the pH-dependent solubility experimental measurement revealed that CFZ precipitation as a hydrochloride salt was highly sensitive to small, but physiologically-relevant variations in pH and chloride concentrations (pHmax=4.5±7.11×10⁻¹⁵, 95% CI=4.5, 4.5; and Ksp=332.3±3.71 μM2, 95% CI=323.1, 341.5) (FIG. 3C). Thus, at the pharmacologically-relevant CFZ concentrations that have been measured in serum (1 to 20 μM), local variations in pH (˜7.2 extracellular; ˜7.4 cytosolic and ˜4.5 lysosomal) and chloride concentrations (˜80 mM extracellular; ˜10-20 mM in cytosol; and ˜50-200 mM in lysosomes) can lead to significant differences in CFZ precipitation and solid-state protonated chemistry in extracellular vs. cytosolic vs. lysosomal microenvironments.

Bathochromic shift in fluorescence of CFZ. CFZ's inherent fluorescence is dependent upon whether it is in free-base form in solution and as a solid crystal (green and red fluorescent; peak excitation: 540-560 nm, peak emission: 560-600 nm) or when present as biocrystals (red and far red fluorescence; peak excitation: 560-600 nm, peak emission: 650-690 nm) (Keswani et al., Cytometry A 87:855-867 2015). To confirm if CFZ-HCl is defined by this reported bathochromicity in fluorescence of the biocrystals, confocal fluorescence microspectroscopy was performed on synthesized CFZ-HCl crystals. The CFZ-HCl crystals also had peak fluorescence activity in a similar spectral range as measured from biocrystals (peak excitation: 560-600 nm, peak emission: 650-690 nm) (FIG. 4a). When visualized using conventional epifluorescence microscopy using the appropriate filters, CFZ-HCl had high far-red fluorescence activity and negligible green fluorescence (FIG. 4a).

Lysosomal accumulation of CFZ in RAW264.7 cells. To demonstrate that CFZ bioaccumulation and eventual biocrystallization is associated with lysosomes, CFZ was incubated with RAW264.7 cells and the type and spatial distribution of fluorescence in the cells was monitored. From the results described above, it was determined that CFZ fluoresces in the green and red fluorescent range while CFZ-HCl would fluoresce in the red and far-red fluorescence range. Green fluorescent, red fluorescent and far-red fluorescent punctuate spots, indicative of vesicular accumulation of CFZ were visible in the cells relative to the control cells (FIG. 4b). Notably, there was minimal colocalization of the far-red fluorescent spots with the green fluorescent spots (Pearson's Colocalization Coefficient=0.30±0.29) while the red fluorescent spots colocalized to a higher degree (p<0.005) with both the green (Pearson's Colocalization Coefficient=0.60±0.22) and far-red (Pearson's Colocalization Coefficient=0.61±0.20) fluorescent spots. This is indicative of the fact that vesicles accumulating CFZ and CFZ-HCl were distinct from each other.

In silico analysis—Computationally defining a lysosome as a localized region of chloride and proton activity. In order to simulate a lysosomal compartment, a well-established and published systems-based mathematical model of lysosomal ion regulation, which assumes a spherical lysosomal vesicle, with radius of 0.34 um, containing 300 V-ATPase and 5000 CLC7 molecules, along with membrane proton permeability of 6×10-5 cm/s and cytoplasmic chloride concentration of 10 mM, was used. These and other lysosomal ion concentration values which are detailed in Table 1 are in agreement with physiological lysosomal conditions (Heuser et al., J Cell Biol 121:1311-1327 1993; Van Dyke, Am J Physiol 265:C901-917 1993; Gambale et al., Eur Biophys J Biophy 22:399-403 1994; Sonawane et al., Journal of Biological Chemistry 277:5506-5513 2002; Alberts, Moelcular Biology of the Cell. Fifth edition. Garland Science, New York 2008) consistent with lysosomal ion homeostasis.

In silico analysis: The role of lysosomal proton transport dominates that of chloride transport on the bioaccumulation of CFZ within lysosomes. To computationally study the roles of these lysosomal membrane proteins on the lysosomal bioaccumulation of CFZ, the number of active V-ATPase and CLC7 molecules per lysosome (resulting in 0 to 100% inhibition of both parameters from baseline physiological input values of 300 and 5000, respectively) were simultaneously varied in the presence and absence of CFZ-HCl accumulating in a dose-dependent manner (0 to 0.01 picomoles/cell/day).

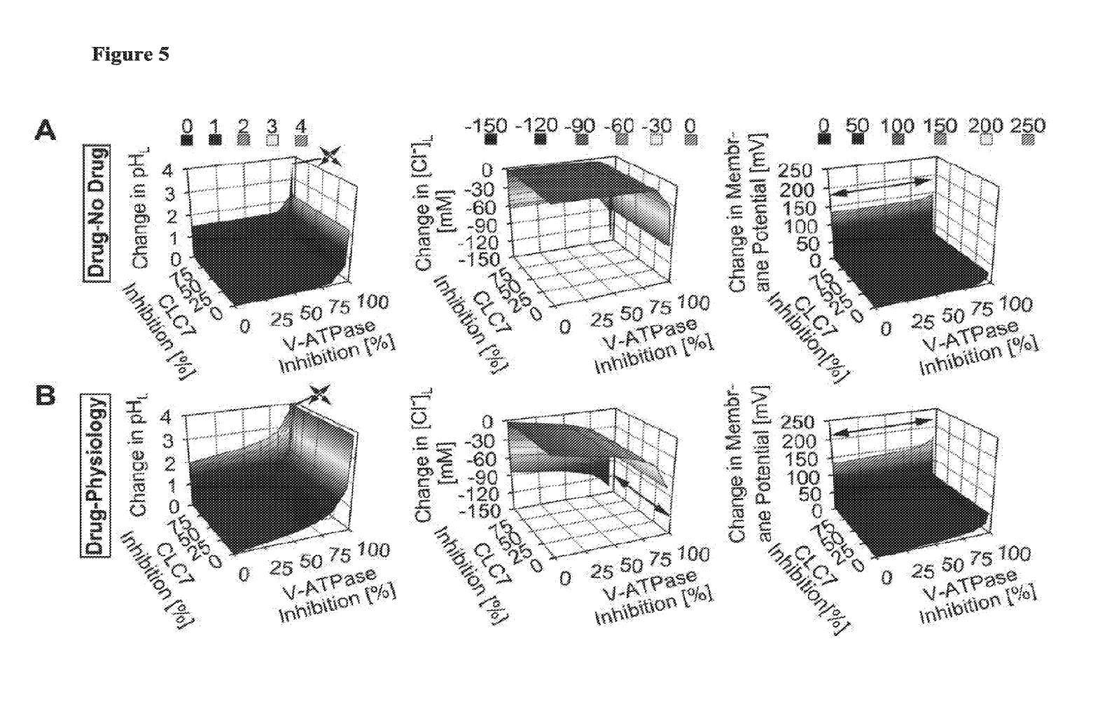

To accommodate lysosomal CFZ-HCl accumulation at the rate of 0.01 picomoles/cell/day, there needed to be at least 9 V-ATPase molecules per lysosome (which corresponds to 97% V-ATPase inhibition), even while maintaining all of the other lysosomal parameters at baseline physiological input values. This strongly indicates the importance of the number of V-ATPase molecules per lysosome in the accumulation of CFZ-HCl, even at the lowest dose of 10×. Accordingly, in the presence of the simultaneous V-ATPase (0 to 97%) and CLC7 inhibition (0 to 100%), the only significant deviation observed in the lysosomal pH, chloride, and membrane potential of the CFZ-HCl containing lysosome from that of the CFZ-HCl free lysosome was at the highest simultaneous V-ATPase (97%) and CLC7 (100%) inhibition, which was reflected by up to >4 pH unit increment in lysosomal pH, >150 mM reduction in lysosomal chloride accumulation, and >250 mV increment in membrane potential (FIG. 5A). However, when studying the physiological aspect of the lysosomal accumulation of 1× CFZ-HCl, it was observed that the number of V-ATPase molecules per lysosome primarily affects the physiological accumulation of the drug in spite of any reduction in the number of CLC7 molecules per lysosome (FIG. 5B). Moreover, it is evident that the physiological perturbation is enhanced in the presence of V-ATPase inhibition, as reflected by up to >4 pH units increment in lysosomal pH, >150 mM reduction in lysosomal chloride, and >250 mV increment in lysosomal membrane potential from their respective baseline physiological values of 4.53 pH unit, 224.6 mM lysosomal chloride, and 0.79 mV lysosomal membrane potential (FIG. 5B).

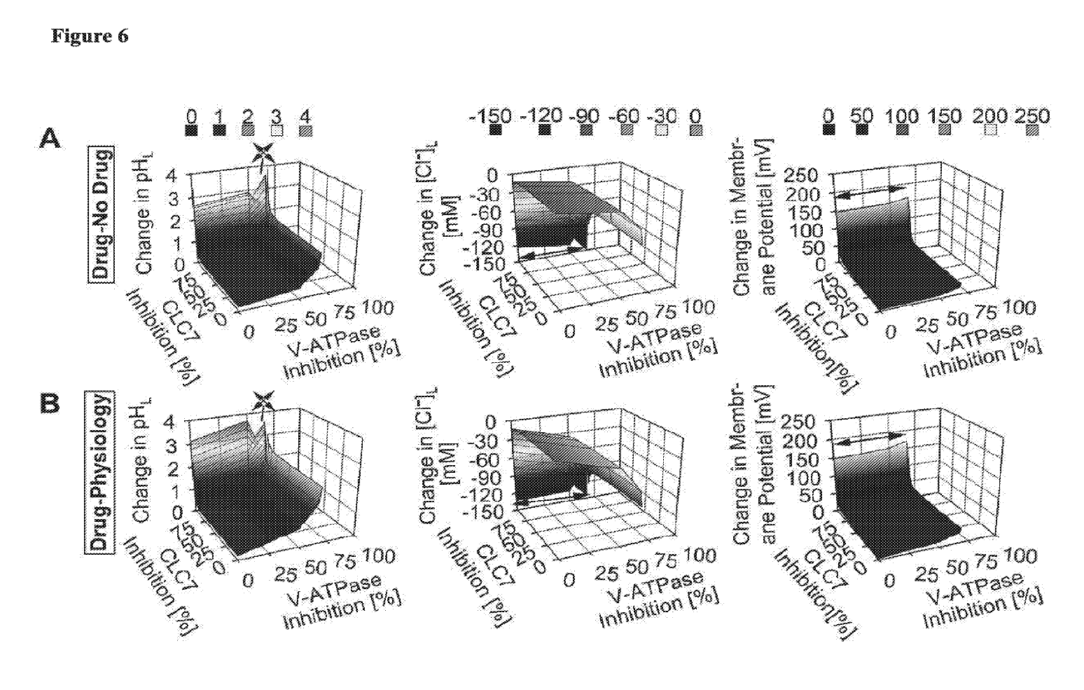

In order to understand the simultaneous effect of the amount of V-ATPase and CLC7 molecules per lysosome on the lysosomal accumulation of higher dose CFZ-HCl, the rate of drug accumulation was increased by 10× while simultaneously varying V-ATPase and CLC7 molecules per lysosome as mentioned before. Consequently, it was observed that there needed to be at least 95 V-ATPase molecules per lysosome (which corresponds to 68.3% V-ATPase inhibition) for the lysosomal accommodation of CFZ-HCl at 10× even when all of the other lysosomal parameters, including the number of CLC7 molecules per lysosome, were kept at their respective baseline physiological input values (FIG. 6A). This indicates that the role of the V-ATPase amount to be especially important than that of the CLC7 amount in the accommodation of such increased drug accumulation. More specifically, >68.3% V-ATPase inhibition showed significant difference between the CFZ-HCl containing and CFZ-HCl free lysosomal pH, Cl, and membrane potential, as well as significant perturbation of lysosomal physiology associated with CFZ-HCl accommodation, which is reflected by >4 pH unit increment in lysosomal pH, >150 mM reduction in lysosomal Cl accumulation, and >250 mV increment in lysosomal membrane potential from their respective baseline physiological values of 4.53 pH unit, 224.6 mM lysosomal chloride, and 0.79 mV lysosomal membrane potential (FIG. 6B).

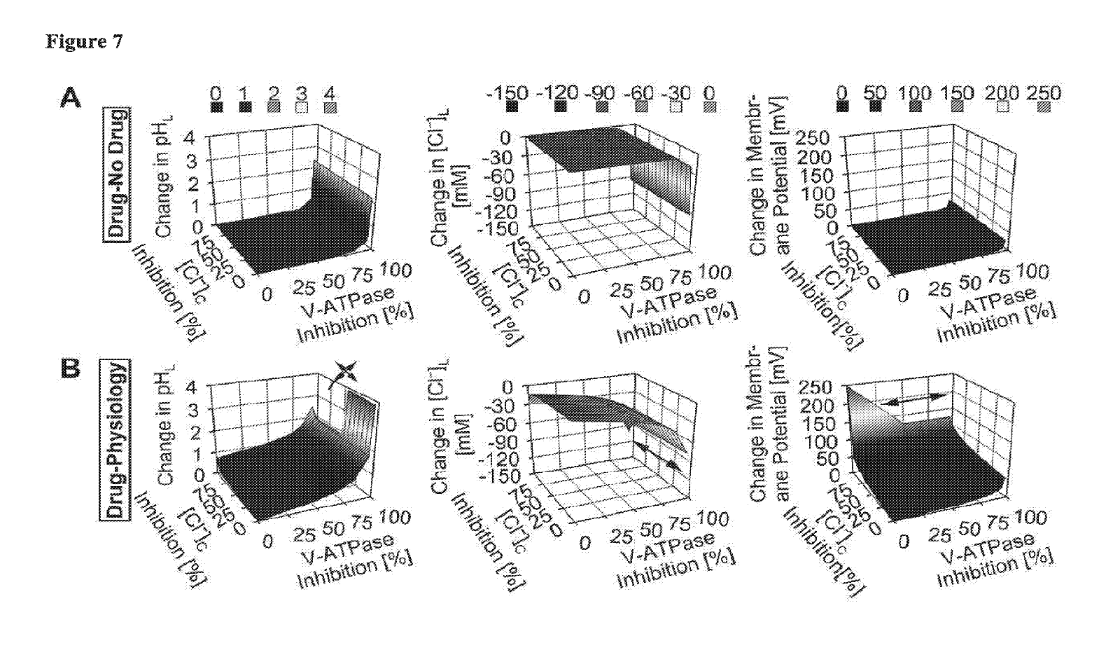

In silico analysis: Comparing the roles of chloride ion versus chloride transporter in V-ATPase mediated bioaccumulation of CFZ within lysosomes. In order to investigate if the level of cytoplasmic chloride instead of the chloride ion transporter has any specific role on the lysosomal accumulation of CFZ-HCl, the cytoplasmic chloride concentration was simultaneously varied along with the number of V-ATPase molecules per lysosome (resulting in 0 to 100% inhibition of both parameters from baseline physiological input values of 300 and 5000, respectively). It was observed that CFZ-HCl accumulation at 0.01 picomol/cell/day did not induce significant change to lysosomal pH, Cl and membrane potential as long as V-ATPase inhibition was <95% (FIG. 7A). However, from the lysosomal physiological perturbation result (FIG. 7B), it is evident that the simultaneous inhibition of cytoplasmic chloride (≥40%) and V-ATPase (≥97%) induced significant perturbation to lysosomal physiology, which was reflected by up to >4 pH unit increment in lysosomal pH, >150 mM reduction in lysosomal chloride accumulation, and >250 mV increment in membrane potential from their respective baseline physiological values of 4.53 pH unit, 224.6 mM lysosomal chloride, and 0.79 mV lysosomal membrane potential (FIG. 7B).

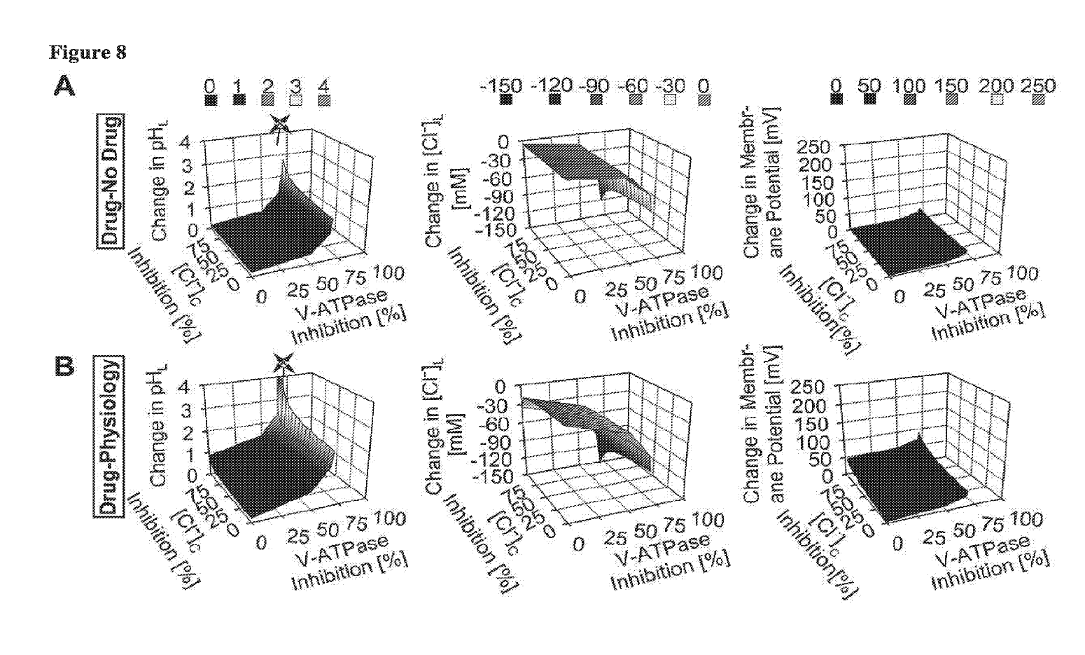

Furthermore, to understand the combined role of both cytoplasmic chloride and V-ATPase number on the accommodation of higher dose of lysosomal CFZ-HCl accumulation, the rate of lysosomal proton and chloride sequestrations by CFZ was adjusted to 10× while lysosomal V-ATPase number per lysosome and cytoplasmic chloride concentration were simultaneously varied as mentioned earlier. Similar to what was previously indicated, CFZ-HCl accumulating at 0.1 picomoles/cell/day cannot be accommodated by the lysosome in the presence of >68.3 V-ATPase inhibition irrespective of any changes in the cytoplasmic chloride concentration (FIG. 8A).

Moreover, it can be observed that for the same amount of V-ATPase molecules per lysosome, the cytoplasmic chloride has more role than the amount of CLC7 molecules per lysosome in the physiological accommodation of dose-dependent lysosomal accumulation of CFZ-HCl (FIGS. 7 and 8). More specifically, in the presence of maximum V-ATPase inhibition (97% in the presence of 1× CFZ-HCl accumulation and 68.3% in the presence of 10× CFZ-HCl accumulation), the role of the amount of CLC7 molecules per lysosome was seen at almost ˜100% CLC7 inhibition in the accommodation of both 1× and 10× CFZ-HCl in the lysosome, while the effect of cytoplasmic chloride was seen at >40% inhibition and >93.3% in the accommodation of 1× and 10× CFZ-HCl, respectively, in the lysosome (FIGS. 7 and 8).

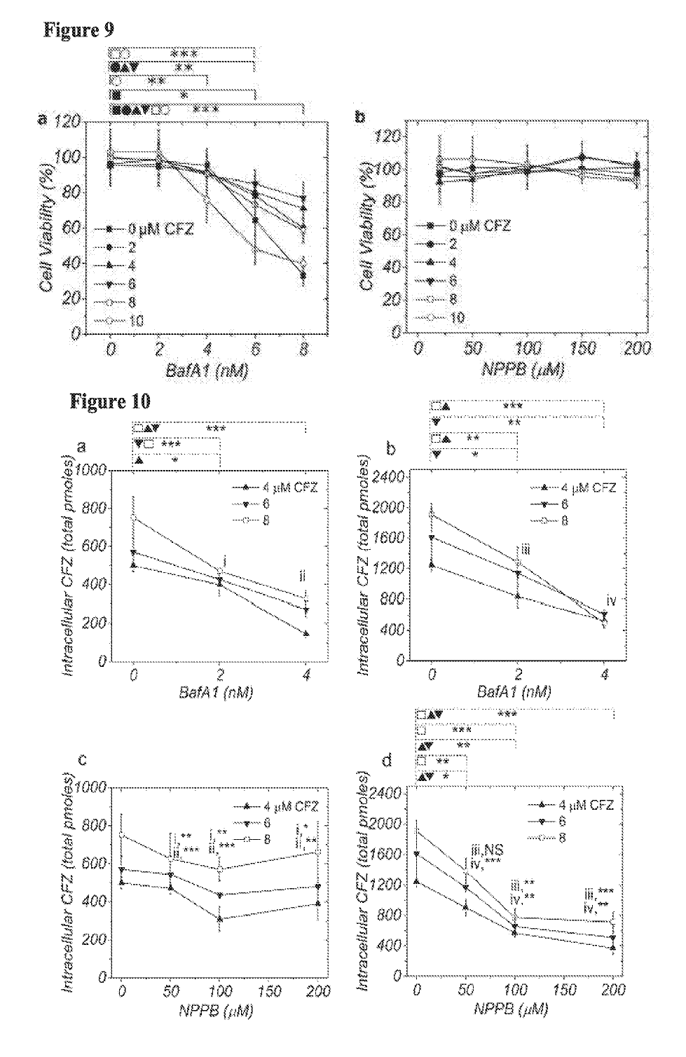

In vitro analysis: Cell viability. To determine the effects of inhibiting proton or chloride transport on CFZ accumulation, cell viability studies using a standard XTT assay were performed to obtain optimal drug exposure conditions (FIG. 9). The incubation of CFZ or NPPB alone had no significant effect on cell viability. However, cell viability dropped significantly when exposed to BafAl alone at 6 nM (65±25%, n=6, p<0.05) and 8 nM (33±6%, n=6, p<0.001). When exposed to NPPB prior to varying concentrations of CFZ, cell viability remained high at >92% for all tested concentrations of NPPB. This is in agreement with simulation result where <99.9% CLC7 inhibition resulted in >93.1% cell viability in the presence of drug accumulation at the rate of 0.01 picomoles/cell/day. Moreover, when exposed to up to 4 nM BafAlprior to up to 8 μM CFZ, cell viability remained a healthy >91%. This is in agreement with simulation results where up to 66.7% V-ATPase inhibition resulted in >87.4% cell viability in the presence of drug accumulation at the rate of 0.01 picomoles/cell/day. However, at BafA1=4 nM and CFZ=10 μM, viability dropped to 76±13%, n=6, p<0.01). Moreover, at BafA1=6 & 8 nM, cell viability decreased to between 48-85% and 33-77% at all concentrations of CFZ. This indicates that the relationship between extracellular CFZ concentration and BafA1 concentration mediated inhibition of V-ATPase activity in affecting cell viability. Accordingly, further analyses for CFZ incubation and accumulation were performed at CFZ=4, 6 and 8μM with BafA1=2 and 4 nM and NPPB=50, 100 and 200 μM.

In vitro analysis (CFZ accumulation). To confirm if accumulation of CFZ was affected in the presence of proton and chloride inhibitors, absorbance spectroscopy was used to determine intracellular drug mass under predetermined conditions that maintained >90% cell viability. Moreover, the quantitative comparison of the experimental drug uptake measurements with the simulation results indicated that in the absence of any inhibitor, the extracellular CFZ concentration that was associated with drug accumulation at a rate approximately equal to 0.01 picomol/cell/day (1×) was 8 μM. Thus, from here onwards, comparison of the wet lab experimental drug uptake measurements with the simulation results in the presence as well as absence of inhibitors are specifically done for wet lab experimental conditions with 8 μM extracellular CFZ only. Furthermore, the following comparisons are reported as a percentage (%) of CFZ accumulation measured in uninhibited cells under the various tested CFZ concentrations and 8 μM CFZ concentration, in the wet lab experimental and simulation studies, respectively.

In the presence of 2 nM BafAl, CFZ accumulation dropped to 80±13% (n=6, p<0.01) at 4 hours and 67±12% (n=6, p<0.01) at 24 hours post CFZ incubation relative to uninhibited cells at an initial concentration of CFZ=4 μM (FIG. 10a). Similarly, CFZ accumulation reduced to 75±11% (4 hours, n=6, p<0.001) and 71±19% (24 hours, n=6, p<0.05) at initial CFZ=6 μM & 63±9% (4 hours, n=6, p<0.001) and 67±12% (24 hours, n=6, p<0.01) at initial CFZ=8 μM, (FIG. 10b) which is in agreement with the simulation result where up to 50% V-ATPase inhibition resulted in 50.3% drug uptake relative to uninhibited or physiological baseline lysosomal parametric conditions.

When cells were exposed to BafA1=4 nM, CFZ accumulation was further reduced relative to the uninhibited cells—29±4% (4 hours, n=6, p<0.001) and 42±6% (24 hours, n=6, p<0.001) at initial CFZ=4 μM, to 47±9% (4 hours, n=6, p<0.001) and 38±9% (24 hours, n=6, p<0.01) at initial CFZ=6 μM and to 43±8% (4 hours, n=6, p<0.001) and 26±4% (24 hours, n=6, p<0.001) at initial CFZ=8 μM, (FIGS. 10a and b) which is in agreement with the simulation result where up to 66.7% V-ATPase inhibition resulted in 33.1% drug uptake relative to uninhibited or physiological baseline lysosomal parametric conditions.

When cells were pre-incubated with various concentrations of NPPB and exposed to CFZ for 4 hours at varying concentrations, no significant change was measured in the accumulation of CFZ in inhibited cells relative to uninhibited cells (FIG. 10c). However, at 24 hours, significant reduction was measured in NPPB pre-treated cells—73±10% (NPPB=50 μM, n=6, p<0.05), 46±5% (NPPB=100 μM, n=6, p<0.01) and 30±7% (NPPB=200 μM, n=6, p<0.001) at CFZ=4 μM; 73±17% (NPPB=50 μM, n=6, p<0.05), 41±12% (NPPB=100 μM, n=6, p<0.01) and 32±12% (NPPB=200 μM, n=6, p<0.001) at CFZ=6 μM & 72±10% (NPPB=50 μM, n=6, p<0.01), 41±7% (NPPB=100 μM, n=6, p<0.001) (FIG. 10d)—which is in agreement with the simulation result where 97.5% CLC7 inhibition resulted in 43.7% drug uptake relative to uninhibited or physiological baseline lysosomal parametric conditions—and 37±7% (NPPB=200 μM, n=6, p<0.001) at CFZ=8 μM (FIG. 10d)—which is also in agreement with the simulation result where 98.8% resulted in 27.1% drug uptake relative to uninhibited or physiological baseline lysosomal parametric conditions.

### EXAMPLE 2

### Macrophage Depletion Reveals an Active Role of the Immune System in Determining the in Vivo Disposition of an Orally-Bioavailable Drug

### Materials and Methods

### Clofazimine Administration to Mice

Mice (4 week old, male C57B16) were purchased from the Jackson Laboratory (Bar Harbor, Me.) and acclimatized for 1 week in a specific-pathogen-free animal facility. Clofazimine (CFZ) (C8895; Sigma, St. Louis, Mo.) was dissolved in sesame oil (Shirakiku, Japan) to achieve a concentration of 3 mg/ml, which was mixed with Powdered Lab Diet 5001 (PMI International, Inc., St. Louis, Mo.) to produce a 0.03% drug to powdered feed mix, and orally administered ad libitum for 4 weeks. A corresponding amount of sesame oil was mixed with chow for vehicle treatment (control). Mice were euthanized via carbon dioxide asphyxiation and exsanguination. Animal care was provided by the University of Michigan's Unit for Laboratory Animal Medicine (ULAM).

### Liposome Administration

In order to deplete tissue macrophages, mice were treated with liposomes containing either 7 mg/mL clodronate or phosphate-buffered saline (PBS) (FormuMax Scientific Inc., Sunnyvale, Calif.) for up to six weeks. Liposomes were injected IP to eliminate macrophages of the liver, spleen, and peritoneal cavity. Mice were initially treated with 200 μL of liposomes, followed by 100 μL injections twice per week to ensure continual depletion of macrophages.

### Macrophage Depletion and CFZ Administration

Mice were fed CFZ or a control diet continuously for a four week period. Following two weeks of feeding, liposome treatment began for two weeks. After completing four weeks of feeding and two weeks of liposome treatment, the mice were sacrificed and tissues were collected.

### Peritoneal Macrophage Isolation

Following euthanasia, a small incision was made in the lower abdomen. The peritoneal cavity was then flushed 10 mL of ice cold PBS containing 5% FBS (Sigma). The peritoneal lavage was centrifuged for 10 min at 400×g, 4° C,and then resuspended in DMEM media (Life Technologies). The cells were then plated onto 4 or 8 chamber coverglass (#1.5, Lab-Tek II, Nunc, Rochester, N.Y.) for imaging. The cells were allowed to attach overnight and then washed with media.

### Alveolar Macrophage Isolation

The trachea was surgically exposed and cannulated with an 18G needle and the lungs were lavaged by instilling DPBS containing 0.5 mM EDTA (Sigma) in 1 ml aliquots for a total of 6 ml. Approximately 90% of the bronchoalveolar lavage (BAL) was retrieved. BAL was then centrifuged for 10 min at 400×g, 4° C., resuspended in RPMI 1640 media (Life Technologies) and the cells were pooled together. The cells were then plated onto 4 or 8 chamber coverglass (#1.5, Lab-Tek II, Nunc, Rochester, N.Y.) for imaging studies. The cells were allowed to attach overnight and then washed with media, enabling the isolation of alveolar macrophages by adherence.

### Sample Preparation for Microscopy

Cryosectioning was carried out using a Leica 3050S cryostat. Samples were sectioned to 5 μm. In preparation for cryosectioning, portions of the organ were removed, immediately submerged in OCT (Tissue-Tek catalog no. 4583; Sakura), and frozen (−80° C.). Immunohistochemistry of F4/80 (Abcam, 1:500 dilution) was performed using Alexa-Fluor 488 (Abcam, 1:500 dilution).

### Biochemical Analysis of CFZ in Tissues

The concentration of CFZ in organs was determined spectrophotometrically. After four weeks of CFZ- or vehicle-diet treatment, mice were euthanized via CO2 asphyxiation, and blood and organs were collected. Tissue (20-30 mg) was homogenized in 500 μL of RIPA buffer, and 350 μL of homogenate was removed drug was extracted with three passes of 1 mL of xylenes. The drug was then extracted from the xylene with three 1 mL passes of 9M sulfuric acid. The concentration of CFZ present in the tissue was then determined using a plate reader at wavelength 450 nm.

Isolation of CLDIs from Mouse Spleen

At 8 weeks post drug feeding, mice were euthanized by carbon dioxide asphyxiation and exsanguination, and spleens were harvested and cut open to prepare tissue homogenate in phosphate-buffered saline (PBS). The tissue was diced and homogenized manually, and collected in PBS. The homogenate was then centrifuged (100×g for 1 minute) to remove large cellular debris. A solution of 10% sucrose in PBS was added to the acquired supernatant and the mixture was centrifuged (100×g). The resulting supernatant was centrifuged (21,000×g for 1 min) to pellet drug inclusions which were then resuspended in 2 ml of 10% sucrose. CLDIs were further purified using a 3-layer discontinuous gradient (50%, 30% and 10% sucrose in PBS) centrifugation method (3200×g for 30 min, no brakes). The CFZ content of the isolated CLDIs was determined using a plate reader at wavelength 450 nm following dissolution in 9M sulfuric acid followed by comparison with calibrated CFZ standards.

CLDI Injection into Peritoneal Cavity

Mice were injected intraperitoneally with 200 μL of either clodronate or PBS liposomes to deplete peritoneal macrophage population. After 48 hours, mice were injected intraperitoneally with 0.250 mg of CLDIs in 500 μL PBS. At the designated time points, mice were euthanized by carbon dioxide asphyxiation and exsanguination, and a peritoneal lavage was performed to collect cells and injected CLDIs.

### Biochemical Analysis of CFZ in Peritoneal Cavity

After collecting the peritoneal lavage, cells were centrifuged (400×g for 10 minutes) and counted. The cells were centrifuged once more, and the pellet was resuspended in 1 mL of DI water. The drug was extracted from the water with three passes of 1 mL xylenes, and the drug was then extracted from the xylenes using three passes of 1 mL of 9M sulfuric acid. The concentration and total mass of CFZ present in the peritoneal cavity was determined using a plate reader at wavelength 450 nm.

### Results

### Macrophage Depletion Impacts Body Weight and Temperature of Drug Treated Animals

During the course of the experiment, the body weight and temperature of the mice were measured daily to track the impact of the loss of drug-sequestering cells on the general health of the mouse. Table 4 shows the final average body weight and temperature for each treatment group following the final measurement. For the PBS treated groups, as well as the control-diet fed clodronate group, there was no statistically significant difference in body weight or temperature. However, in the clodronate-treated and CFZ-fed group, mice weighed on average five grams less than their PBS-treated and control-diet fed counterparts, and had a body temperature that was on average a full degree Celsius lower (p<0.05, One-way ANOVA).

TABLE 4
Mouse Body Weight and Temperature

| | Average Body | Average Body |
|---|---|---|
| Treatment | Weight (n = 3-4) | Temperature (n = 3-4) |
| Control + PBS | 26.2 ± 1.2 g | 34.1 ± 0.3° C. |
| Control + Clodronate | 26.6 ± 1.1 g | 34.2 ± 0.4° C. |
| CFZ + PBS | 25.7 ± 0.8 g | 34.3 ± 0.3° C. |
| CFZ + Clodronate | 21.2 ± 1.4 g | 32.9 ± 0.8° C. |

### Clodronate Depletion Impacts Peritoneal Macrophages, but not Alveolar

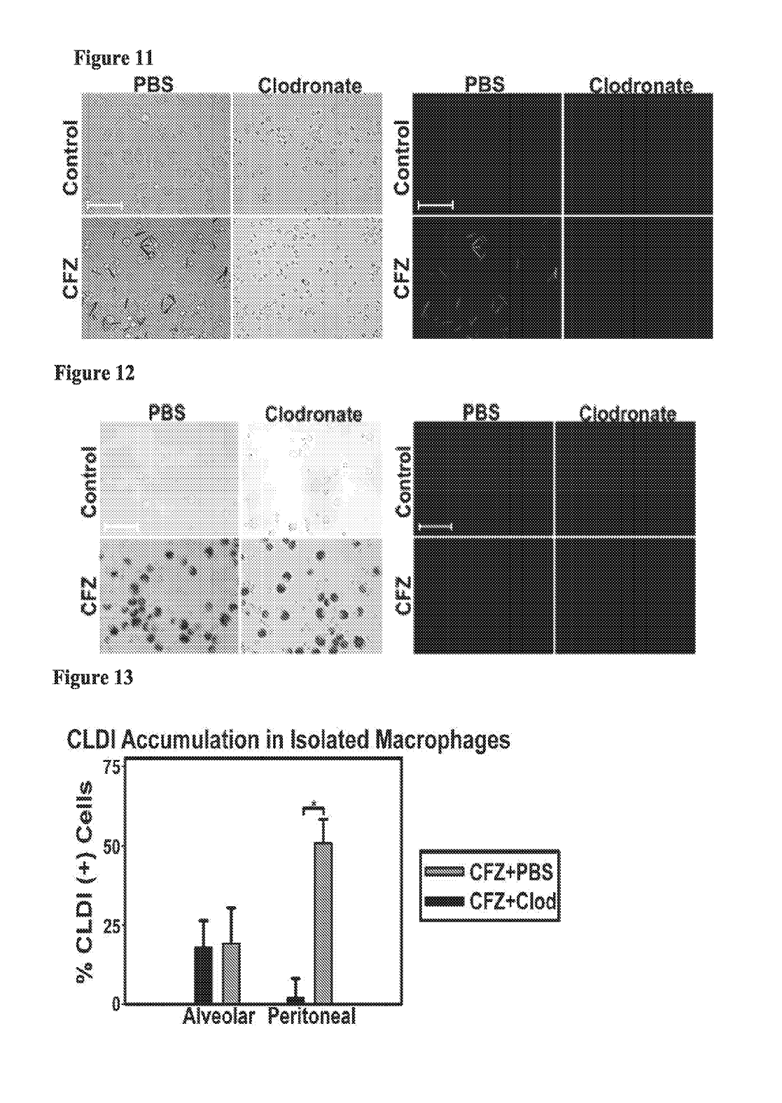

Due to the route of administration, macrophages of the peritoneal cavity are significantly reduced by treatment with clodronate. As a result of the death of these cells, it is hypothesized that CLDIs will not remain stable, leading to an overall reduction of intracellular crystals in the peritoneal cavity. The reduction in macrophages can be seen in FIG. 11, which compares the peritoneal exudate from each treatment group, and is quantified in Table 5, which includes the average cell counts obtained following a peritoneal lavage. In the PBS-treated group, peritoneal cells readily attach to the surface of the cover glass and spread out, and can be easily distinguished from the other cells found in the cavity, such as B-cells. When mice are injected with clodronate, there is a significant reduction in the presence of macrophages in the peritoneal cavity, as shown in FIG. 11.

TABLE 5
Cell counts obtained from peritoneal lavage of mice

| Treatment | Peritoneal lavage cell count (n = 3-4 per group) |
|---|---|
| Control + PBS | 2.4 × 10⁷ ± 2.1 × 10⁷ |
| Control + Clodronate | 1.5 × 10⁶ ± 9.6 × 10⁵ |
| CFZ + PBS | 7.7 × 10 [symbol] ± 1.9 × 10⁶ |
| CFZ + Clodronate | 1.1 × 10 [symbol] ± 4.1 × 10⁵ |

[symbol]

indicates data missing or illegible when filed

In the PBS-CFZ fed group, approximately 50% of the macrophages present contain at least one intracellular CLDI, consistent with previous reports. The loss of macrophages in the peritoneal cavity resulted in a significant reduction of CLDI containing cells (FIG. 13), indicating that their stabilization may be macrophage-specific, rather than cell-specific.

Macrophages of the lung, or alveolar macrophages, have also been implicated in the disposition of CFZ and in the accumulation of CLDIs. In order to determine if the clodronate was impacting CLDI accumulation within the lung, a similar analysis was performed on cells collected following a broncho-alveolar lavage (BAL). In both the clodronate and PBS treated groups, similar numbers of macrophages were collected. In both the PBS and clodronate treated, CFZ fed mice, similar percentages of CLDI containing cells were detected, indicating that clodronate treatment did not impact macrophages of the lung (FIG. 13).

Clodronate Treatment Lowers Drug Accumulation in both Liver and Spleen

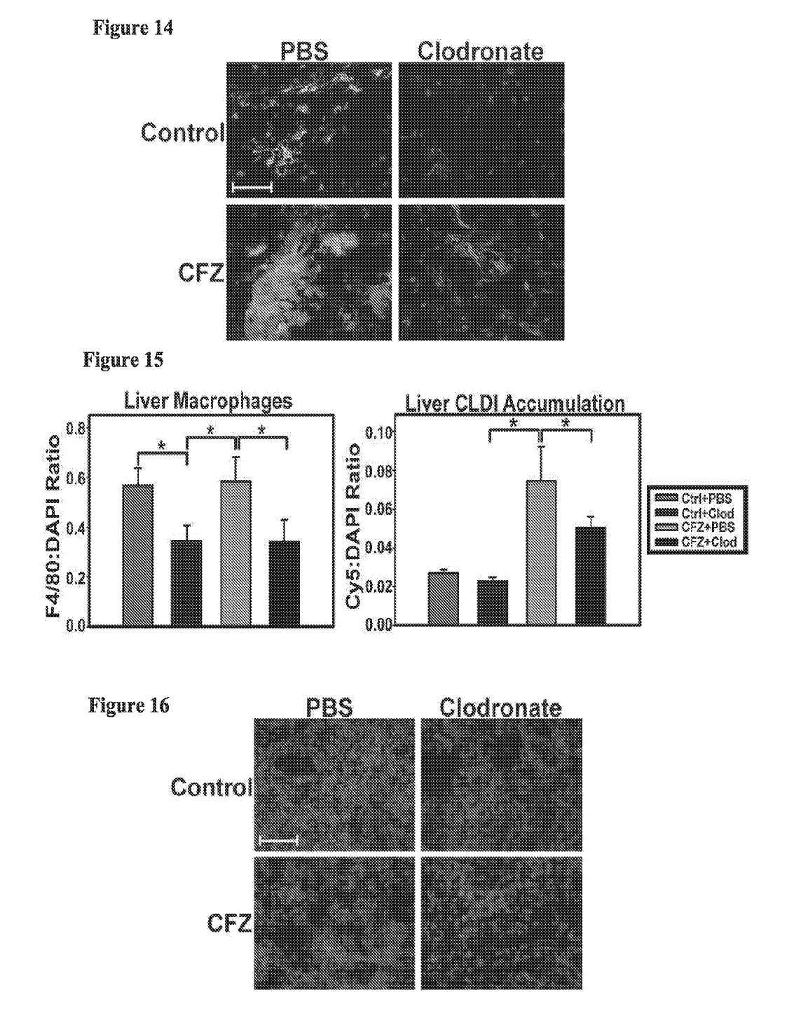

Both the liver and spleen accumulate high amounts of crystal-like drug inclusions (CLDIs) with resident tissue macrophages throughout CFZ therapy. In order to quantify the reduction in macrophages and drug accumulation in the liver and spleen, immunohistochemical staining for the macrophage marker F4/80 was used in conjunction with the intrinsic Cy5 fluorescence of CLDIs. In both the PBS treated groups, F4/80 positive Kupffer cells are numerous and readily detectable. In the CFZ treated group, one can see that the bright red CLDIs tend to be found within groups of these cells as well, indicating that their stability is dependent on their action (FIG. 14). Quantitative cytometric analysis of liver cryosections revealed a 42±15% reduction in macrophages as a result of clodronate treatment (p<0.001, Two-tailed T-test) and a 32±6% reduction in the prevalence of CLDIs (p=0.015, Two-Tailed T-Test) (FIG. 15).

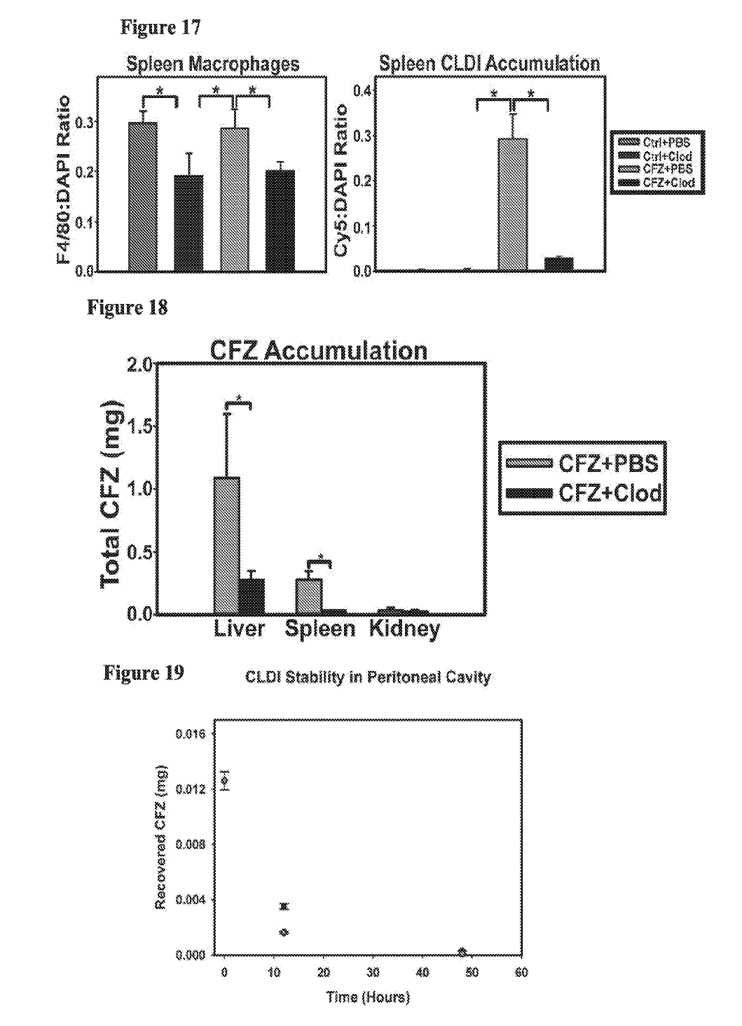

Similar to the liver, the spleen also contains a large population of macrophages, known as red-pulp macrophages, which accumulate significant amounts of CFZ during treatment. In the PBS treated spleen, red pulp macrophages are readily detectable. Similar to what is seen in the liver, clodronate treatment results in a reduction in the population of these cells (FIG. 17). In the spleen, there was a 33±6% reduction in total red pulp macrophages (p=0.001, Two-Tailed T-Test). Due to the loss of drug-sequestering red-pulp macrophages, there was a 90±2% reduction in CLDI accumulation in the spleen (p<0.001, Two-Tailed T-Test) (FIG. 18).

Because clofazimine can exist in the tissue as both an physiologically insoluble crystal and as a soluble free base, the total amount of drug sequestered within the organ was then determined. In the liver of clodronate-treated mice, there was a 75±7% reduction in the total amount of drug found in the organ (p=0.047, Two-Tailed T-Test). Additionally, in the spleen, there was an 88±7% reduction in drug (p=0.006, Two-Tailed T-Test). In an organ which was not affected by clodronate treatment, the kidney, there was no difference in total amount of drug found (p=0.77, Two-Tailed T-Test) (FIG. 18).

Macrophages Impart Stability to Injected CLDIs

Long-term treatment with liposomal clodronate or PBS may impact lysosomal physiology by altering pH, which may have an effect on the ability of CFZ to accumulate within these cells. In order to overcome this possibility, an experiment was performed to determine the impact macrophage loss has on CLDI stability. Mice were injected I.P. with 200 μL of liposomal clodronate or PBS, and 48 hours later, were treated with 0.250 mg of isolated CLDIs in 500 μL of PBS. Peritoneal lavages were performed at each time point to determine internalization of injected CLDIs, and to measure the amount of drug within the peritoneal cavity. In the macrophage-depleted mice, there is a significant reduction in the total recoverable CFZ relative to that of the PBS treated group (FIG. 19).

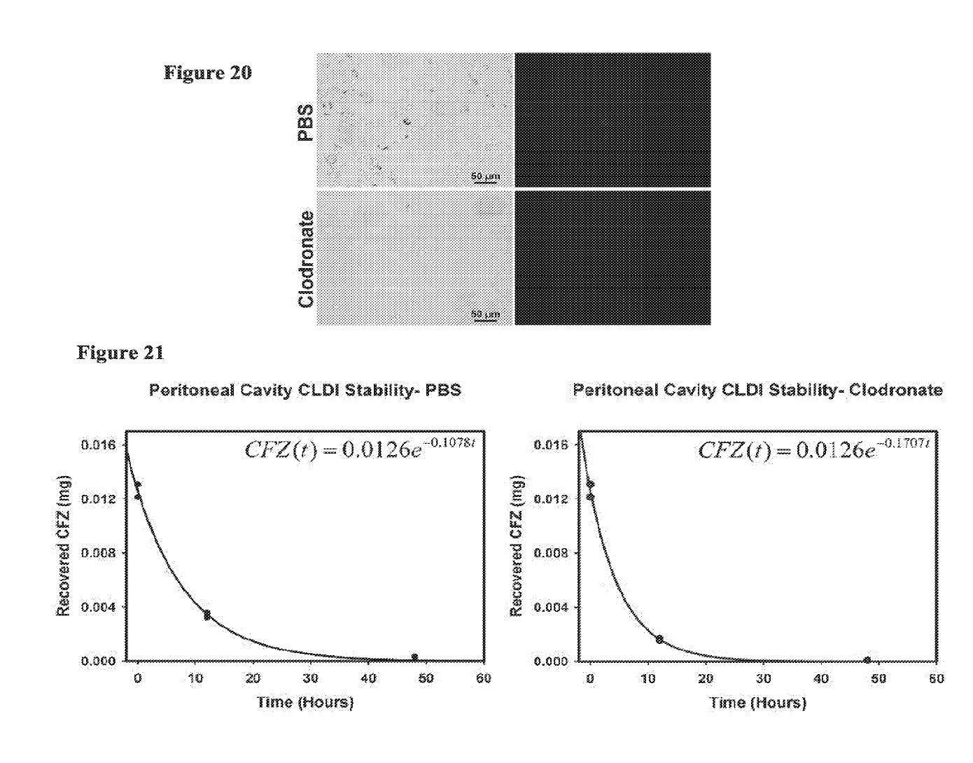

Microscopic analysis of the peritoneal lavage following 48 hours reveals that, in both treatment groups, CLDIs are internalized by macrophages of the peritoneal cavity (FIG. 20). However, the reduction in the population of macrophages due to the clodronate leads to a lower amount of drug being internalized, as measured in FIG. 19. As a result of this, the extracellular crystals are destabilized and dissolve. Using the mass of CFZ that was recovered, an exponential regression analysis was performed to determine the half-life of injected CLDIs that are stabilized by macrophages versus those which are not, revealing a significant increase in the degradation constant k, as well as a significant reduction in the half-life (FIG. 21).

In conclusion, chemical depletion of drug-sequestering macrophages resulted in a concomitant reduction both the accumulation of physiologically insoluble aggregates of drug, or CLDIs, within the liver, spleen, and peritoneal cavity, as well as a reduction in the total amount of drug within the liver and spleen. This provides evidence that the immune system can play a role in the disposition of an orally-bioavailable drug. Specifically, these experiments show that macrophages actively sequester drug, and that without these cells, crystal-like drug inclusions are not able to accumulate. Additionally, without macrophages present in the peritoneal cavity, injected biocrystals dissolve and become destabilized at a significantly faster rate than when these cells are present, indicating that the stabilization of CFZ biocrystals is a macrophage-dependent phenomenon.

### EXAMPLE 3

### Methods

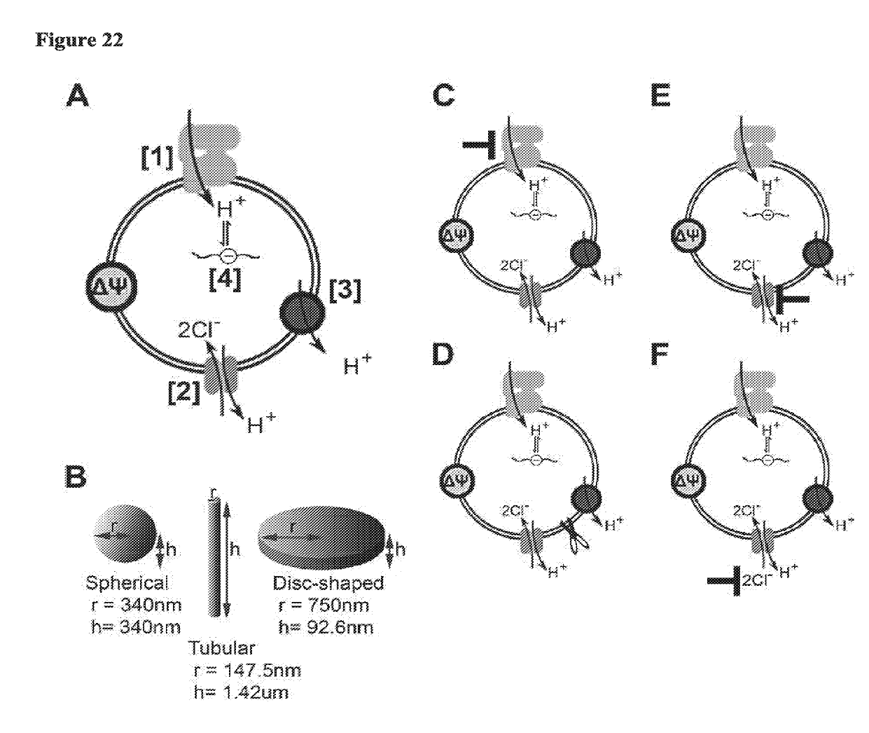

The established, systems-based mechanistic model of lysosomal ion transport (Graves AR, et al., Nature. 2008;453(7196):788-92; Ishida Y, et al., J Gen Physiol. 2013;141(6):705-20) was used to capture the physiological consequences of drug-induced lysosomal stress (FIG. 22). The transportation of ions to and from the lysosome induces charge (ΔQ) and membrane potential difference (ΔΨ) between the lysosome and the cytoplasm. By definition, membrane potential difference, which simply is referred hereon as membrane potential, across a given membrane is the difference between the potential of one side of the membrane and that of the other side of the membrane (the lysosome and the cytoplasm in this case). It can be calculated using the following relationship between the charge of a given ion(s) and capacitance, assuming that the lipid bilayer of lysosomal membrane can serve as a parallel plate capacitor:

$$ΔΨ = \frac{Δ Q}{{C}^{′}} \qquad ( 1 )$$

Where (C′) is the specific bilayer capacitance of the lysosomal membrane per unit area of the lysosomal surface area, which represents the measure of the net charge transported (ΔQ), in units of Coulomb, from one side of the membrane to the other resulting in membrane potential (ΔΨ) across the membrane, in units of mV. For biological membranes, (C′) has been experimentally approximated to 1 μF/cm² (Fricke H. J Gen Physiol. 1925;9(2):137-52; Fenwick E M, et al., J Physiol. 1982;331:599-635).

Charge on a mole of a monovalent ion is represented by Faraday's constant (F), which equals 96485 Coulomb/mol. Moreover, depending on the charge and valence (Zi) of the monovalent ion “i”, (F) will be (+) or (−), where the earlier is for a cation and the latter for an anion. Thus, using (F) one can obtain the charge of the ions in the cytoplasm and the lysosome from the cytoplasmic and lysosomal ion concentration, respectively, in units of Coulomb.

$$Δ Q = F * ( {\sum }_{i} {{Z}_{i} [ i ]}_{C} {V}_{C} - {\sum }_{i} {{Z}_{i} [ i ]}_{L} {V}_{L} ) \qquad ( 2 )$$

With [i_{C}] and [i_{L}] being the net concentration in the cytoplasm and lysosome in units of Molar, respectively. Both terms are multiplied by their respective compartmental volume to convert the units of molar to mol. In both compartments, the net concentration comprises of permeable and impermeable ions.

Furthermore, a physiological sign convention that the membrane potential is more positive or negative if there is more cation or anion, respectively, in the internal compartment (lysosome), than in the external compartment (cytoplasm) was adopted. Thus, equation 2 is re-written as:

ΔQ=F*(ΣᵢZᵢ[t]_{L}V_{L}−ΣᵢZᵢ[t]_{C}V_{C}) (3)

More specifically, the following relationship is obtained to define the ion content in the cytoplasmic compartment:

$${\sum }_{i} {[ i ]}_{C} {V}_{C} = {\sum }_{i} {( {[ i ]}_{C} {V}_{C} )}_{f} - {\sum }_{i} {( {[ i ]}_{C} {V}_{C} )}_{o} \qquad ( 4 )$$

Where the subscript (f) denotes the final value of the permeable ions in units of mole, and the subscript (o) denotes the initial value of the impermeable ions in units of mole. However, because the cytoplasmic volume is comparatively big, it is assumed that the concentration of cytoplasmic ions remains more or less constant. Thus, Σᵢ([i]_{C}V_{C})ₒ=0

Similar to equation 4, the following relationship for the case of lysosomal ion content is used:

$${\sum }_{i} {[ i ]}_{L} {V}_{L} = {\sum }_{i} {( {[ i ]}_{L} {V}_{L} )}_{f} - {\sum }_{i} {( {[ i ]}_{L} {V}_{L} )}_{o} \qquad ( 5 )$$

Thus, by substituting the terms in equation 3 with those in equations 4 and 5, one obtains:

$$Δ Q = F * ( {( {\sum }_{i} {{Z}_{i} [ i ]}_{L} {V}_{L} )}_{f} - {\sum }_{i} {{Z}_{i} ( {[ i ]}_{L} {V}_{L} )}_{o} ) \qquad ( 6 )$$

In addition, because the lysosomal volume is treated as a parameter, equation 6 can be re-written as:

$$Δ Q = {FV}_{L} * ( {( {\sum }_{i} {{Z}_{i} [ i ]}_{L} )}_{f} - {\sum }_{i} {{Z}_{i} ( {[ i ]}_{L} )}_{o} ) \qquad ( 7 )$$

Moreover, for the purpose of distinguishing notations, Σᵢ([i]_{L})ₒ) is replaced by (B). This term is known as Donnan particles, which is represented in units of molar. Thus, equation 7 is re-written as:

$$Δ Q = {FV}_{L} * ( {( {\sum }_{i} {{Z}_{i} [ i ]}_{L} )}_{f} - B ) \qquad ( 8 )$$

Thus, by plugging equation 8 into equation 1, membrane potential is equated as;

$$ΔΨ = \frac{{FV}_{L}}{{C}^{′}} * ( {( {\sum }_{i} {{Z}_{i} [ i ]}_{L} )}_{f} - B ) \qquad ( 9 )$$

As previously mentioned, because the capacitance is a measurement per unit area of the lysosomal surface, equation 9 is multiplied by the total lysosomal surface area (S), in units of cm2, to obtain total capacitance per lysosome, hence the total membrane potential.

$$ΔΨ = \frac{{FV}_{L}}{{C}^{′} S} * ( {( {\sum }_{i} {{Z}_{i} [ i ]}_{L} )}_{f} - B ) \qquad ( 10 )$$

Furthermore, the final luminal ion concentrations [i_{L}]_{f} can be classified into cations and anions:

$$ΔΨ = \frac{{FV}_{L}}{{C}^{′} S} * [ {( {\sum }_{i} {{Z}_{i} [ cations ]}_{i} )}_{f} + {\sum }_{i} {{Z}_{i} [ anions ]}_{i} ) - B ] \qquad ( 11 )$$

In addition, the Donnan particles (B), in units of Molar, are explicitly defined using the initial lysosomal contents, including the net change in the intrinsic surface potential:

$$B = {[ {H}^{+} ]}_{L , initial} + {[ {K}^{+} ]}_{L , initial} + {[ {Na}^{+} ]}_{L , initial} - {[ {Cl}^{-} ]}_{L , initial} - \frac{CS}{{FV}_{L}} * \{ ( {Ψ}_{in} - {Ψ}_{out} ) + {Ψ}_{initial} \} \qquad ( 12 )$$

With the subscript (initial) denoting the fixed initial luminal content, (Ψᵢₙ) and (Ψₒᵤₜ) being the intrinsic surface potentials for inner and outer leaflets in units of mV, respectively, of the lysosomal membrane, as estimated in the literature (Ishida et al., 2013, supra), (Ψᵢₙᵢₜᵢₐₗ) being the initial membrane potential, which is set to 0 mV for the purpose of maintaining initial electroneutrality.

Substituting Equation 12 into Equation 11 gives:

$$ΔΨ = \frac{{FV}_{L}}{{C}^{′} S} * \{ {[ {H}^{+} ]}_{L , final} - {[ {Cl}^{-} ]}_{L , final} - {[ {H}^{+} ]}_{L , initial} + {[ {Cl}^{-} ]}_{L , initial} \} + {Ψ}_{in} - {Ψ}_{out} \} \qquad ( 13 )$$

With the subscript (final) denoting the luminal contents at a given time “t”.

Ion Transportation

Briefly, in this model, V-ATPase is an electrogenic proton pump which lowers the lysosomal pH by working against a proton motive force build-up that arises from membrane potential and proton concentration gradient. To capture this phenomenon, the rate of proton influx (J_{HVATP}) by a single V-ATPase molecule per lysosome per second was incorporated to the model as a call-in function based on the instantaneous transmembrane pH gradient (ΔpH) and membrane potential (ΔΨ), Equation 14. This rate was multiplied by the total number of active V-ATPase molecules in a lysosome (NVATP) (Graves et al., 2008, supra; Ishida et al., 2013, supra) to obtain the total number of protons inserted into the lysosome in units of molecules per second (Hpump), Equation 14.

Hₚᵤₘₚ=N_{VATP}*J_{H}_{_{VATP}} (ΔpH, ΔΨ) (14)

Following proton-pumping by the V-ATPase, membrane potential increases in the absence of some other mechanism that dissipates the increment. Physiologically, membrane potential is dissipated by the efflux of cations from the lysosomes, or the influx of anions. In the latter case, CLC7 plays a dominant role (Graves et al., 2008, supra). Accordingly, the model represents the rate of proton removal per second (J_{CLH}_{_}_{CLC7}), as well-elaborated in previously published models (Ishida et al., 2013, supra), by a single CLC7 molecule per lysosome as a function of chloride concentration gradient dictated by cytoplasmic and lysosomal chloride concentrations (ClC and ClL, respectively), and electrical gradient dictated by change in membrane potential (ΔΨ), Equation 15. This rate was multiplied by the total number of CLC7 molecules per lysosome (NClC7) (Ishida et al., 2013, supra) in order to obtain the total amount of protons removed in units of molecules per second by CLC7 (HClC7) ,Equation 15.

H_{ClC7}=N_{ClC7}*J_{CLH}_{_{ClC7}}(Δph, Cl_{L}, Cl_{C}, ΔΨ) (15)

In addition to CLC7, membrane potential dissipation is also facilitated by the process of proton transportation from the lysosome to the cytoplasm. To capture the passive diffusion of protons across the semi-permeable lysosomal membrane, the Goldman-Hodgkin-Katz (GHK) flux equation (Weiss TF. Cellular Biophysics: Transport: MIT Press; 1996) was used. GHK flux equation was derived to model the flux of an ion “i” (jᵢ) dictated by both chemical (dC/dX) and electrical potential (dT/dX) gradients across a biological membrane:

$${j}_{i} = - {Z}_{i} {u}_{i} RT * ( \frac{dC}{dX} + {Z}_{i} C \frac{F}{RT} \frac{dΨ}{dX} ) \qquad ( 16 )$$

Where (Z) is charge and valence of the ion “i”, (C) is the ion concentration, (R) is gas constant, (T) is absolute temperature, 0 Kelvin, and (μᵢ) is the electro-kinetic ion mobility and is further related to the diffusion coefficient (Dᵢ) of the ion by the following Einstein relation:

$${u}_{i} = {D}_{i} * \frac{{Z}_{i} F}{RT} \qquad ( 17 )$$

Assuming constant electric field across a homogenous membrane with thickness of (l), the above equation is integrated at steady state to give the flux of ion “i” across the membrane using boundary conditions where at x=0, the ion concentration=Cₒ (ion concentration before transportation at one side of the membrane) and at x=(l), the ion concentration=Cl (ion concentration following transportation to the other side of the membrane):

$${j}_{i} = \frac{{u}_{i}}{l} * ΔΨ * \frac{{C}_{o} - ( {C}_{1} * {e}^{- ZY} )}{1 - {e}^{- ZY}} \qquad ( 18 )$$

Where (Y) is the membrane potential difference (ΔΨ) normalized by (RT/F) for cells at room temperature, 25° C., which equals 25.69 mV (53):

$$Y = \frac{ΔΨ F}{RT} \qquad ( 19 )$$

Thus, substituting terms in equation 18 by the ones in equations 17, one obtains:

$${j}_{i} = \frac{{D}_{i} {Z}_{i} F}{RTl} * ΔΨ * \frac{{C}_{o} - ( {C}_{1} * {e}^{- ZY} )}{1 - {e}^{- ZY}} \qquad ( 20 )$$

Moreover, equation 20 can be further simplified using the relationship in equation 19:

$${j}_{i} = \frac{{D}_{i} {Z}_{i} Y}{l} * \frac{{C}_{o} - ( {C}_{1} * {e}^{- ZY} )}{1 - {e}^{- ZY}} \qquad ( 21 )$$

Furthermore, the diffusion coefficient can be written in terms of the permeability of the ion across the membrane using the following equation:

$${P}_{i} = \frac{{K}_{i} {D}_{i}}{l} \qquad ( 22 )$$

Where K₁ is the water-membrane partition coefficient of the ion “i” and measures the solubility of the ion in lipids. This term is set to 1 for either a co-ion or a counterion since the pore size of an ion membrane transporter is generally large and the partition coefficient of an ion in a pore approaches 1 as the pore size increases (Buyukdagli S, et al., Phys Rev E Stat Nonlin Soft Matter Phys. 2010;81(4 Pt 1):041601).

Thus, by plugging equation 22 into equation 21,

$${j}_{i} = {P}_{i} YZ * \frac{{C}_{o} - ( {C}_{1} * {e}^{- ZY} )}{1 - {e}^{- ZY}} \qquad ( 23 )$$

Because equation 23 gives the ion flux over a single area in units of molar per second, the rate is multiplied by the total lysosomal surface area (S) to determine the total ion flux, as follows:

$${j}_{i} = {SP}_{i} YZ * \frac{{C}_{o} - ( {C}_{1} * {e}^{- ZY} )}{1 - {e}^{- ZY}} \qquad ( 24 )$$

Accordingly, the total amount of free proton that can be transported from the lysosome to the cytoplasm (Hₗₑₐₖ) in units of molar per second (which is further converted into molecules per second by multiplying by Avogardo's number (Nₐᵥ)) follows the derivation of equation 24 and thus can be written as:

$${H}_{leak} = ( {SP}_{{H}^{+}} YZ * \frac{{10}^{- p {H}_{L}} - ( {10}^{- p {H}_{C}} * {e}^{- ZY} )}{1 - {e}^{- ZY}} ) * {N}_{av} \qquad ( 25 )$$

With (pH⁺) being the proton permeability in units of cm/s, (pH_{L}) is the lysosomal luminal pH in units of pH units used to calculate the free lysosomal proton (10^{-pHL}) in units of Molar, (pH_{C}) is the cytoplasmic pH in units of pH units used to calculate the free cytoplasmic proton (10^{-pHC}) in units of Molar, Z, S, and Y are the same terms as previously described.

Surface Concentration

Using an equation derived from the previously defined GHK flux equation, the concentrations of the transported ions are further modified based on the surface membrane potential exposed to either the cytoplasm or the lysosomal compartment (Hille B. Ionic Channels of Excitable Membranes. 2nd Edition. Sunderland, Mass. : Sinauer Associates, Inc.; 1992). In the case where net current flow is zero, the GHK equation that defines the membrane potential, which in this case is also defined as Nernst potential (ΔΨi) of a given ion “i” is presented as:

$$ΔΨ = {ΔΨ}_{i} = \frac{- ZRT}{F} ln \frac{{C}_{i , 0}}{{C}_{i , 1}} \qquad ( 26 )$$

With Cᵢ being the concentration of the ion “i” in units of molar and the position of the ion are denoted by the subscripts 0 and 1.

Thus, by inserting the intrinsic internal leaflet potential (Ψin) in units of mV into equation 26, one can calculate the concentration of a given ion at the membrane surface facing the lysosomal compartment according to the following relationship:

$${ΔΨ}_{i , in} = \frac{- ZRT}{F} ln \frac{{C}_{i , in}}{{C}_{i , L}} \qquad ( 27 )$$

With (C_{i,in}) being the internal concentration of a given ion at the membrane surface facing the lysosomal compartment (inner leaflet) in units of molar and (C_{i,L}) is the concentration of the same ion in the non-membrane region of the lysosomal compartment in units of molar.

Similarly, by inserting intrinsic external or outer leaflet potential (Ψₒᵤₜ) in units of mV into equation 26, one can calculate the concentration of a given ion at the membrane surface facing the cytoplasmic compartment according to the following relationship:

$${ΔΨ}_{i , out} = \frac{- ZRT}{F} ln \frac{{C}_{i , out}}{{C}_{i , C}} \qquad ( 28 )$$

With (C_{i,out}) being the external concentration of a given ion at the membrane surface facing the cytoplasmic compartment (outer leaflet) in units of molar and (C_{i,c}) is the concentration of the same ion in the non-membrane region of the cytoplasmic compartment in units of molar.

Governing Equations

The aforementioned ion flux expressions are used to generate a kinetic model to monitor the time-dependent changes in lysosomal proton and chloride molecules per second as described by Equations 29 and 30:

$$\begin{matrix}\frac{{dH}^{+}}{dt} = {H}_{pump} - {H}_{ClC 7} - {H}_{leak} & ( 29 ) \\ \frac{{dCl}^{-}}{dt} = 2 * {N}_{ClC} * {J}_{Cl , {H}_{{ClC}_{7}}} ( Δ pH , {Cl}_{L} , {Cl}_{C} , ΔΨ ) & ( 30 )\end{matrix}$$

The coefficient “2” in Equation 30 indicates that CLC7 inserts 2 Cl ions for every proton it removes (in 2:1 stoichiometric ratio), which was used in published model (Ishida et al, 2013, supra) based on analysis of experimental studies (Graves et al., 2008, supra).

One can further define the time-dependent change in pH by converting the total amount of lysosomal proton in units of molecules per second (as represented in equation 29) into units of molar per second by dividing the terms in equation 29 by lysosomal volume and Avogadro's number. Moreover, the molar per second unit is converted into units of pH unit per second by dividing the entire term by the buffering capacity (β), which itself is represented in units of molar per pH unit :

$$\frac{d pH}{dt} = \frac{( - {H}_{pump} + {H}_{ClC 7} + {H}_{leak} )}{V * {N}_{av} * β} \qquad ( 31 )$$

The lysosomal lumen buffering capacity of the Donnan particles entraps protons and thus dictates lysosomal pH. It is experimentally measured by introducing a strong acid or base (in units of Molar) that can induce change of 1 pH unit. Thus, the physiological accumulation of weakly basic compounds, such as cationic amphiphilic and lysomotropic drugs, has an insignificant effect on the buffering capacity, as the amount of proton they sequester from the lysosome is fully released upon the hydration of the protonated drug, hence maintaining mass balance mediated physiological lysosomal pH. For simplicity purpose, although the buffering capacity varies with pH, it was set to a constant according to literature values (Ishida et al., 2013, supra; Gekle M, Silbernagl S. Pflugers Arch. 1995;429(3):452-4).

Model Parameterization

The model has 22 parameters: 6 were adjustable and the remaining 16 were fixed. Fixed parameters were set to published values, which give rise to physiological lysosomal function (Ishida et al., 2013, supra; Grabe M, Oster G. J Gen Physiol. 2001;117(4):329-44) and are referred hereon as “baseline input values”. These parameters include lysosomal radius of 340 nm with volume and surface area corresponding to a spherical lysosomal vesicle (obtained from electron microscopy data (Van Dyke R W. Am J Physiol. 1993;265(4 Pt 1):C901-17)), and the associated organellar ions and ion transporters, which include 300 V-ATPase molecules per lysosome (estimated from microscope data analysis and wet lab experimental data fitting (Ishida et al., supra; Gambale F, et al., Eur Biophys J Biophy. 1994;22(6):399-403; Heuser J, et al., J Cell Biol. 1993;121(6):1311-27), membrane proton permeability of 6×10-5 cm/s (estimated from wet lab experimental data fitting (49)), cytoplasmic chloride concentration of 10 mM (Sonawane N D, et al., Journal of Biological Chemistry. 2002;277(7):5506-13; Alberts B J, et al., Moelcular Biology of the Cell. Fifth edition: Garland Science, New York; 2008. 1601 p.), and initial lysosomal ion concentrations estimated to equal that of extracellular ions. A lysosome with the entire model parameter values set to baseline input values (e.g., when lysosomal parameters are treated as fixed parameters) is referred to as an “unperturbed lysosome”.

Adjustable parameters are those that were varied from their respective baseline input values in order to simulate the lysosomal stressors. Therefore, lysosomes consisting of one or multiple of these parameters are referred hereon as “perturbed lysosomes”. Moreover, the lysosomal ion stressors reported here are associated with stressors inducing variations in lysosomal membrane proton permeability, cytoplasmic chloride concentration, and V-ATPase and CLC7 molecules per lysosome, whereas the lysosomal morphology stressors are associated with stressors inducing variations in lysosomal surface area and volume. By random selection, some of these parameters were varied using the modeling software's, which is known as Berekely Madonna (BM), parametric plot feature where the initial and final values of the chosen parameter are inserted in specific arithmetic or geometric interval. Alternatively, parameters that were not varied using BM's parametric plot feature were manually varied in arbitrarily chosen intervals. This distinctive method of parametric variation are described below.

TABLE 6
Model Parameters

| | | Baseline Input | Range of Input | |
|---|---|---|---|---|
| Symbol | Description | Value | Value | Units |
| pH_{C} | Cytosolic pH | 7.2 | Fixed | pH unit |
| pH_{L} | Luminal pH | 7.4 | Fixed | pH unit |
| [Cl⁻]_{C} | Cytosolic | 10 | 1 × 10⁻⁵-10 | mM |
| | chloride | | | |
| | concentration | | | |
| [Cl⁻]_{L} | Luminal chloride | 110 | Fixed | mM |
| | concentration | | | |
| [Na⁺]_{L} | Luminal sodium | 145 | Fixed | mM |
| | concentration | | | |
| [K⁺]_{L} | Luminal | 5 | Fixed | mM |
| | potassium | | | |
| | concentration | | | |
| [H⁺]_{L} | Luminal proton | 0 | Fixed | mM |
| | concentration | | | |
| P_{H}⁺ | Membrane | 6 × 10⁻⁵ | 1.38 × 10⁻⁷-6 | cm/s |
| | proton | | | |
| | permeability | | | |
| V | Lysosomal | 1.65 × 10⁻¹⁶ | 2.88 × 10⁻¹⁷-1.65 × | L |
| | volume | | 10⁻¹⁶ | |
| S | Lysosomal | 1.45 × 10⁻⁸ | 1.45 × 10⁻⁸-6.28 × | cm² |
| | surface area | | 10⁻⁸ | |
| C′ | Specific bilayer | 1 | Fixed | μFarad/cm² |
| | capacitance | | | |
| β | Buffering | 40 | Fixed | mM/pH |
| | capacity | | | |
| N_{VATP} | V-ATPase | 300 | 1 × 10⁻⁴ -1.3 ×10⁵ | |
| | number | | | |
| N_{ClC7} | CLC7 number | 5000 | 1 × 10⁻⁴ -5000 | |
| Ψₒᵤₜ* | Outer surface | −50 | Fixed | mV |
| | potential | | | |
| Ψᵢₙ* | Inner surface | 0 | Fixed | mV |
| | potential | | | |
| CLC_Cl | CLC7 Cl⁻ | 2 | Fixed | |
| | stoichiometry | | | |
| CLC_H | CLC7 H⁺ | 1 | Fixed | |
| | stoichiometry | | | |
| R | Gas constant | 8.314 | Fixed | J · K⁻¹ · mol⁻¹ |
| T | Absolute | 0 | Fixed | Kelvin |
| | temperature | | | |
| F | Faraday's | 96485 | Fixed | J/volt |
| | constant | | | |
| Nₐᵥ | Avogadro's | 6.02 × 10²³ | Fixed | molecules/mol |
| | number | | | |

Baseline input values are literature values (Van Dyke R W. Am J Physiol. 1993;265(4 Pt 1):C901-17; Gambale et al, 1994, supra; Heuser et al, 1993, supra; Sonawane et al, 2003, supra; Alberts et al., 2008, supra) representing physiological lysosomes and are in agreement with previously published model (Ishida et al., 2013, supra; Grabe et al., 2001, supra).

* estimated intrinsic surface potentials for inner (Ψin) and outer (Ψout) leaflets of the lysosomal membrane accounted for when modeling membrane transporter mediated dynamic lysosomal and cytoplasmic ion concentrations at the surface (Grabe et al., 2001, supra).

Simulating Drug Induced Changes in Lysosomal Morphology

To study how drug-induced changes in lysosomal morphology affect ion homeostasis, simulations were performed in lysosomes of different surface areas and volumes. First, lysosomes were modeled as perfect spheres. Assuming there are approximately around 100 lysosomes in a cell, which occupy 1% of cellular volume, the volume of a single lysosome was set to 1.65×10⁻¹⁶ L. For a spherical vesicle, this volume corresponds to a lysosomal radius of 0.34 um and a surface area of 1.45×10+cm².

For comparison, tubular lysosomes (represented in FIG. 22B) possessing different radii in the range 40 nm to 270 nm and heights in the range 585 nm to 5.73 um were modeled using a range of volumes 2.88×10⁻¹⁷ L to 1.34×10⁻¹⁶ L. The dimensional relationship between the tubular radius and height, at constant lysosomal surface area of 1.45×10⁻⁸ cm² (equivalent to the surface area of a spherical lysosome of 0.34 um in radius), was calculated using cylindrical equation (V=πr²h, where r is radius and h is height). These morphologies are consistent with measurements by other publications (Swanson J, et al., J Cell Biol. 1987;104(5):1217-22; Knapp PE, et al., J Cell Sci. 1990;95 (Pt 3):433-9).

Next, to mimic disc-shaped lysosomes as have been reported in different cell types under other biologically relevant conditions (Agarwal R, et al., Proc Natl Acad Sci U S A. 2013;110(43):17247-52; Doshi N, et al., Proc Natl Acad Sci U S A. 2009;106(51):21495-9; Mitragotri S, et al., Nat Mater. 2009;8(1):15-23), lysosomal radius in the range 422 nm to 10 um and height in the range 0.5 nm to 294.9 nm were modeled using a rage of lysosomal surface area 1.90×10⁻⁸ cm² to 6.28×10⁻⁶ cm². The dimensional relationship between the disc-shaped lysosomal radius and height, at constant lysosomal volume of 1.65×10⁻¹⁶L (equivalent to the volume of a spherical lysosome of 0.34 um in radius, as previously mentioned in the study of spherical lysosomes), was calculated using cylindrical equation (S=2πr²+2πrh, where r is radius and h is height).

Simulating Stress Tolerance Following Drug Induced Changes in Lysosomal Morphology

To understand how lysosomes possessing different structural and functional characteristics may respond differently to drugs, the number of V-ATPase and membrane proton permeability were individually varied. These two particular lysosomal parameters are referred to “stress tolerance inducers”. Thus, the number of V-ATPase molecules per lysosome was increased from physiological baseline input value of 300 to 1.3×10⁵ in arbitrarily chosen intervals, while proton permeability was decreased from physiological baseline input value of 6×10⁻⁵ cm/s to 1.38×10⁻⁷ cm/s in arbitrarily chosen intervals. These ranges of input values for both V-ATPase number and proton permeability allowed us to quantitatively compare and contrast the relationship of lysosomal surface area to the number of V-ATPase molecules and membrane proton permeability per lysosome. For example, different fold increments in lysosomal surface area (say, 20- or 400-fold) were chosen and in one simulation at a time set the corresponding lysosomal surface area as an input. So, in the case of a lysosome with a 20-fold lysosomal surface area expansion, its surface area was set to 2.9×10⁻⁷ cm². Then, to study the effect of the number of V-ATPase molecules on this expanded lysosome, parametric simulation was performed for the V-ATPase number ranging 0 to 6000, which corresponds to 0 to 20-fold increment in V-ATPase number per lysosome.

Simulating Drug Induced Lysosomal Stress

Parametric simulations were run to study the effects of the following four drug-induced stresses on lysosomal physiology: V-ATPase inhibition, CLC7 inhibition, lysosomal membrane permeabilization, and decreased membrane potential dissipation based on lower cytoplasmic chloride concentration (FIG. 22).

More specifically, membrane proton permeability was varied from physiological baseline input value of 6×10⁻⁵ cm/s to 6 cm/s in geometric intervals of 1.78, the number of V-ATPase molecules per lysosome was manually varied from 0 to physiological value of 300 in arbitrarily chosen intervals, the number of CLC7 molecules per lysosome was varied using parametric feature of BM from 0 to physiological value of 5000 in geometric intervals of 2.09, and the cytoplasmic chloride concentration was manually varied from 0 to physiological value of 10 mM in arbitrarily chosen intervals. For cytoplasmic chloride concentration, and V-ATPase and CLC7 numbers the individual ranges of input values were chosen to represent the corresponding inhibition range from 0% to 100%, where 0% is no change from respective physiological input value; hence no stress is induced, whereas 100% is maximum change from respective physiological input value; hence maximum inhibition related stress is induced. The inhibition range for a given lysosomal parameter was calculated by comparing each of the individual input values from the aforementioned given range (Adjusted Input Value) to the respective physiological input value, which as previously mentioned it also referred to as “baseline input value” as follows:

$$\% Inhibition = \frac{Baseline Input Value - Adjusted Input Value}{Baseline Input Value} * 100 \% \qquad ( 32 )$$

Simulating individual lysosomal ion stressors. The effects of the lysosomal ion stressors were individually studied in spherical and different sized tubular lysosomes by performing parametric simulation of the four parameters mentioned in the previous subsection (using the same ranges of input values with corresponding intervals) while setting the lysosomal volume and surface area input values to correspond to either a spherical or tubular lysosomal geometry, as indicated in the earlier subsection. For example, in the case of introducing CLC7 inhibitor to a spherical lysosome, lysosomal volume and surface area were set to 1.65×10⁻¹⁶ L and 1.45×10⁻⁸ cm², respectively. Then the CLC7 number was varied from 0 to 5000 in geometric intervals of 2.09, as previously indicated. Similar approach was applied when studying stressor effect in tubular lysosomes except the input value for lysosomal volume was varied from 2.88×10⁻¹⁷ L to 1.65×10⁻¹⁶ L in arbitrarily chosen intervals, while fixing lysosomal surface area at 1.45×10⁻⁸ cm².

Simulating combinations of lysosomal ion stressors. Moreover, the effects of the various combinations of the aforementioned lysosomal ion stressors were studied in spherical, tubular, and disc-shaped lysosomes. The stressor combinations included V-ATPase inhibition-CLC7 inhibition, V-ATPase inhibition-Cytoplasmic Cl depletion, V-ATPase inhibition-membrane proton permeabilization, CLC7 inhibition-Cytoplasmic Cl depletion, CLC7 inhibition-membrane proton permeabilization, Cytoplasmic Cl depletion-membrane proton permeabilization. The ranges of values, with the associated specific or arbitrarily chosen intervals for each parameter mentioned in the previous sections were applied here as well. For example, to simulate the administration of V-ATPase inhibition-CLC7 inhibition to a spherical lysosome, lysosomal volume and surface area input values were set to 1.65×10⁻¹⁶ L and

1.45×10⁻⁸ cm², respectively to adjust the lysosomal geometry as a sphere. Then the CLC7 number was varied from 0 to 5000 in geometric interval of 2.09. Simultaneously, the V-ATPase number was—one simulation at a time—manually set to an arbitrarily chosen value within the range 0 to 300.

Calculating the Effects of Lysosomal Stressors and Tolerance on Lysosomal Physiology

The set of differential equations elaborated earlier was solved by numerical integration in Berkeley Madonna (BM) using Rosenbrock stiff solver as numerical integrator. Time-plot simulations were performed to obtain final lysosomal pH, Cl and membrane potential readout values for an unperturbed lysosome, where all of the parameters are set to physiological values. These variables were specifically chosen as they are the most dynamic and direct indicators of lysosomal physiology in the model. Thus, these values (pH=4.53, total Cl=224.6 mM, and total membrane potential=0.79 mV) are referred to as “baseline readout values” as they are associated with the unperturbed physiological lysosome.

Similarly, when performing each of the aforementioned parametric lysosomal stress and stress tolerance simulations, final lysosomal pH, Cl, and membrane potential variables were chosen as readouts as a function of either a specific lysosomal stressor or stress tolerance inducer in order to generate two-dimensional data. Then, the 2D dataset was saved as a Notepad file and exported to Excel for further analysis. The final lysosomal pH, Cl, and membrane potential values were subtracted from their respective physiological baseline readout values mentioned earlier. This was performed in order to determine the net effect of the lysosomal stressor or the stress tolerance inducer on lysosomal physiology based on the changes in lysosomal pH, Cl and membrane potential.

In addition, the attainment of steady-state readout values was checked by increasing simulation runtime (>24 hr). Furthermore, mass balance was checked and attained for conditions where the thermodynamic limit of V-ATPase proton pump (up to 4.6 pH unit gradient or Gibb's free energy of ˜63 kBT associated with saturated hydrolysis of 3 ATPs (66)) was maintained.

Generating 3 Dimensional Data

Two dimensional data mentioned in the previous section was obtained while—one simulation at a time—manually varying the input value of an additional lysosomal parameter to a specific value within the assigned range. Individual 2D datasets associated with a specific input value of the additional lysosomal parameter were saved as a Notepad file. Then, all of the datasets were collectively exported to Excel and categorized in an individual matrix format for each of the pH, Cl, and membrane potential datasets. To delineate the additional lysosomal parameter in the matrix, which define the 3rd dimension, either the row or column (whichever is not associated with the range of values for the other lysosomal parameter that makes up the 2D dataset) of the matrix dataset was assigned with the corresponding range of values of the additional parameter. Then, either the final lysosomal pH, Cl, or membrane potential values in the matrix dataset were subtracted from the respective physiological baseline readout value mentioned earlier. This was performed in order to determine specific mechanisms of the net effect of the combination of the lysosomal stressors on lysosomal physiology based on the changes in the lysosomal pH, Cl and membrane potential. The individual changes in lysosomal pH, Cl and membrane potential values were used (Z-axis) along with two lysosomal stressors (X and Y axes) to generate a 3D surface plot using SigmaPlot®.

### Results

Using a mechanism-based mathematical model of lysosomal ion transport, computational simulations were performed to reveal how drug-induced variations in one or more ion transport mechanism influence lysosomal physiology, as captured by lysosomal pH, Cl, and membrane potential. To facilitate interpretation of these results, lysosomal ion stressors and lysosomal morphology stressors were considered separately. The earlier directly perturb lysosomal ion transportation, while the latter directly perturb lysosomal morphology.

As elaborated in the following subsections, the effects of the aforementioned lysosomal stressors on lysosomal physiology were considered in the context of 1) biologically-determined variations in lysosomal morphology, 2) drug induced changes in lysosomal volume and surface area, 3) changes in lysosomal volume and surface area as may happen during endocytosis or exocytosis.

Modeling the Effects of Individual Alteration of Lysosomal Proton and Chloride Transportation on Lysosomal Physiology

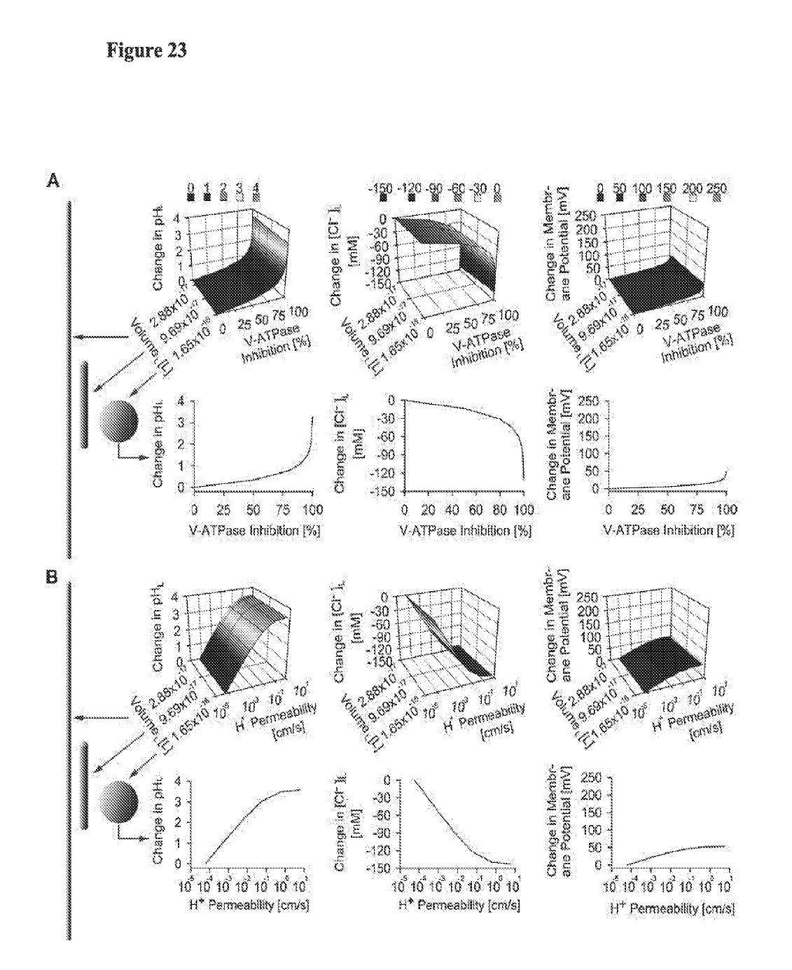

First, the effects of stress inducers that perturb lysosomal ion transporters (V-ATPase, CLC7, and membrane proton permeability) and ion content (cytoplasmic chloride) were studied in the context of a spherical lysosome (FIG. 23A). Among the stressors modeled, maximum reduction in the number of V-ATPase molecule per lysosome induced significant physiological perturbation in a spherical lysosome, as evidenced by the following changes: 3.55 pH unit increment in lysosomal pH and 142.2 mM reduction in lysosomal Cl accumulation from their respective baseline values of 4.53 pH unit and 224.6 mM of an unperturbed lysosome (FIG. 23A). A similar physiological perturbation was observed following the modeling of the effect of increasing the lysosomal membrane proton permeability on lysosomal ion homeostasis (FIG. 23B). This indicates that lysosomal stressors that directly affect lysosomal proton level by perturbing either proton influx or efflux have similar effect not only on lysosomal pH, but also on lysosomal chloride homeostasis. To the contrary, CLC7 and cytoplasmic Cl stressors induced comparatively less perturbation to the overall lysosomal physiology as they only affected lysosomal pH and membrane potential when chloride transport or concentration were completely abolished.

To understand how the same lysosomal ion stressors affect lysosomal ion homeostasis of different lysosomal morphologies, the lysosomal ion stressors were each varied along with lysosomal volume stressors that simultaneously reduced lysosomal radius and volume from an unperturbed lysosomal radius of 340 nm and a corresponding volume of 1.65×10⁻¹⁶ L, to 40 nm and 2.88×10⁻¹⁷ L, respectively. These dimensional changes resulted in tubular lysosomes, which are known to be mainly prevalent before and during the initiation of material uptake (Knapp et al., 1990, supra). Accordingly, they are generally narrower and more elongated than spherical lysosomes (Swanson et al., 1987, supra). In spite of the lysosomal radius and volume differences, tubular lysosomes have the same surface area as spherical lysosomes. This assumption is reasonable because balance in cellular membrane material is maintained as a result of the regulation of membrane trafficking upon tubular lysosome mediated exocytosis of endocytosed material from the subcellular spherical lysosome to the plasma membrane. Thus, based on the results, the lysosomal ion stressors showed to have similar effects on the physiology of tubular lysosome as that of spherical lysosome, reflected by the very similar changes in lysosomal pH, chloride, and membrane potential (FIG. 23).

Modeling the Effects of Lysosomal Expansion on Lysosomal Ion Homeostasis

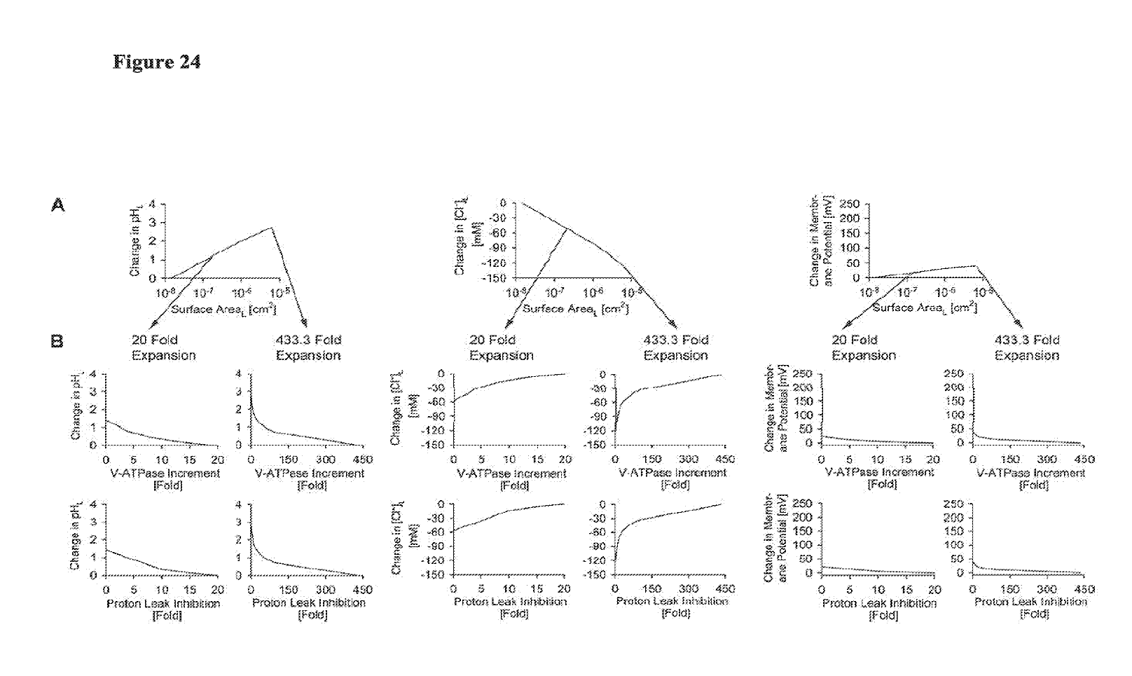

Next, the effect of lysosomal swelling on lysosomal physiology was modeled by simultaneously increasing lysosomal radius and surface area while maintaining a fixed lysosomal volume of a spherical lysosomal morphology, which equals 1.65×10⁻¹⁶ L. Starting from a non-perturbed spherical lysosome, which is 340 nm in radius with a corresponding surface area of 1.45×10⁻⁸ cm², the lysosomal radius and the corresponding surface area were expanded up to 10 um and 6.28×10⁻⁶ cm² (433.3 fold change), respectively. Such lysosomal swelling led to a 2.74 pH units increment in lysosomal pH, 125.6 mM reduction in lysosomal Cl accumulation, and 39.9mV increment in lysosomal membrane potential (FIG. 24A). Thus, lysosomal expansion compromised the maintenance of physiological lysosomal ion homeostasis, similar to that of V-ATPase inhibition and membrane proton permeabilization in tubular and spherical lysosomes (FIG. 23).

Identifying the Mechanisms of Lysosomal Stress Tolerance in Response to Lysosomal welling

To understand the mechanism by which cells can withstand stress generated from simultaneous lysosomal radius and surface area expansion, key parameters that mediate lysosomal stress tolerance (referred to as “lysosomal stress tolerance inducers”) were identified by varying V-ATPase numbers and membrane proton permeability. For this purpose, V-ATPase number and membrane proton permeability were individually increased and decreased, respectively, 0 to 20 and 0 to 433.3 fold from their respective baseline input values. This was performed in order to maintain a constant number of proton influx and efflux mediated by V-ATPase and membrane proton permeability, respectively, for a given lysosomal surface area. IT was observed that physiological ion homeostasis was fully restored when either the number of V-ATPase molecules or the membrane proton permeability was kept proportional to the lysosomal surface area expansion at a fixed lysosomal volume of 1.65×10⁻¹⁶ L (FIG. 24B).

Modeling the Effects of Altering Multiple Lysosomal Ion Transport Pathways on Spherical, Tubular and Disc-Shaped Lysosomal Physiology

Next, the mechanism by which various combinations of lysosomal ion stressors exert their effects on the physiology of lysosomes with distinct morphology was investigated. Different combinations of various ranges of lysosomal parameters to represent V-ATPase-CLC7 stressors, V-ATPase-cytoplasmic chloride stressors, V-ATPase -membrane proton permeability stressors, and CLC7-cytoplasmic chloride stressors were used. These stressor combinations were simulated to study their effects on the physiology of spherical, tubular, and disc-shaped lysosomes.

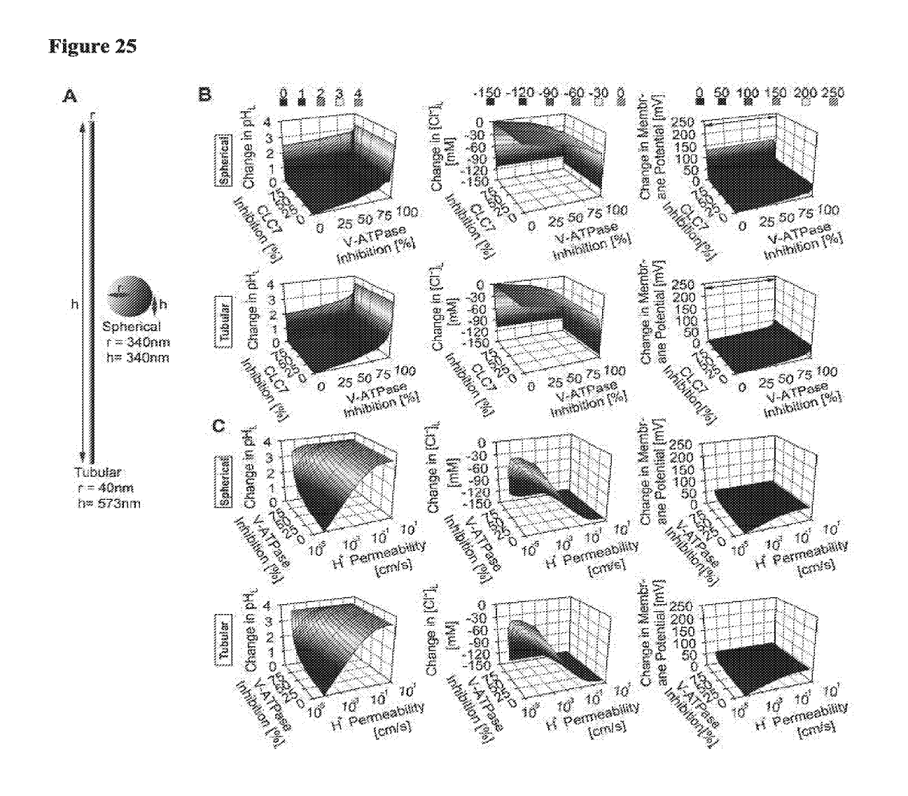

For all of the lysosomal morphologies, maximum alteration of either CLC7 or cytoplasmic chloride parameters in combination with alteration in either V-ATPase number or membrane proton permeability generally induced perturbation of lysosomal physiology due to the build-up of membrane potential by up to >250 mV (FIGS. 25B, 26 and 27).

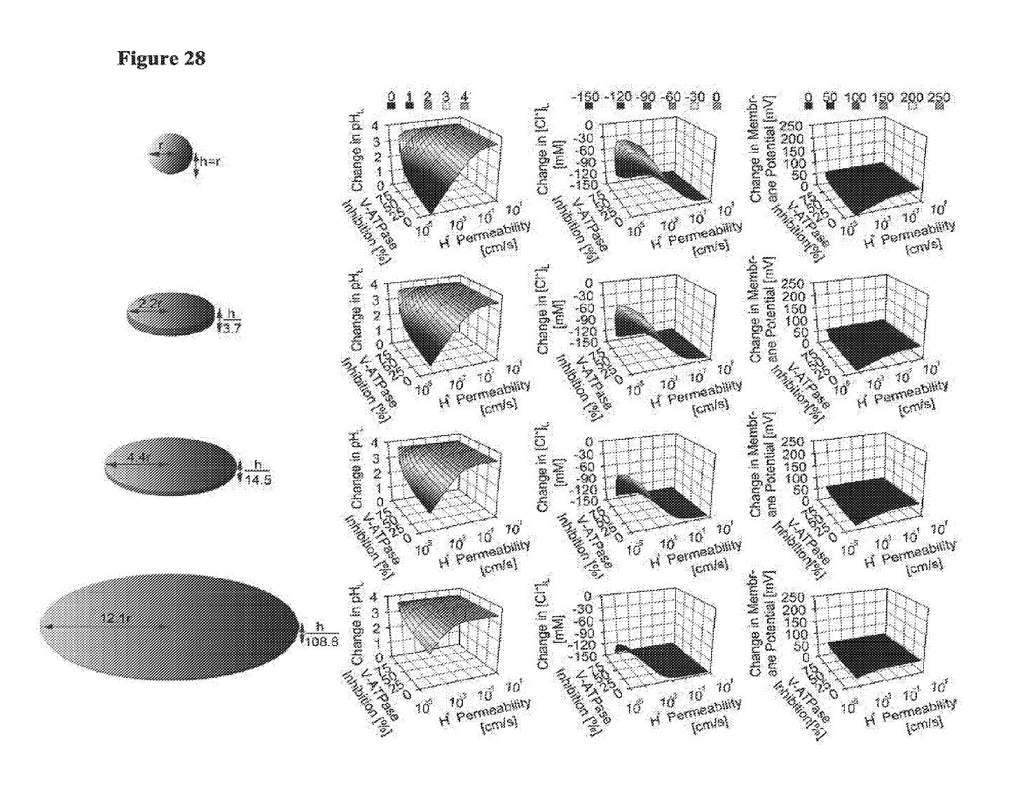

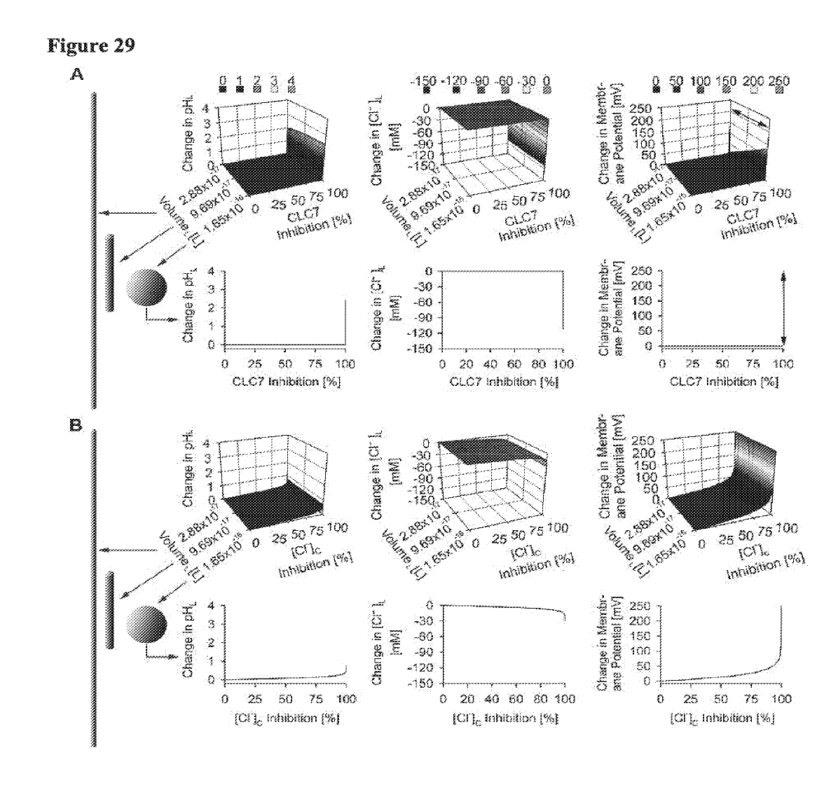

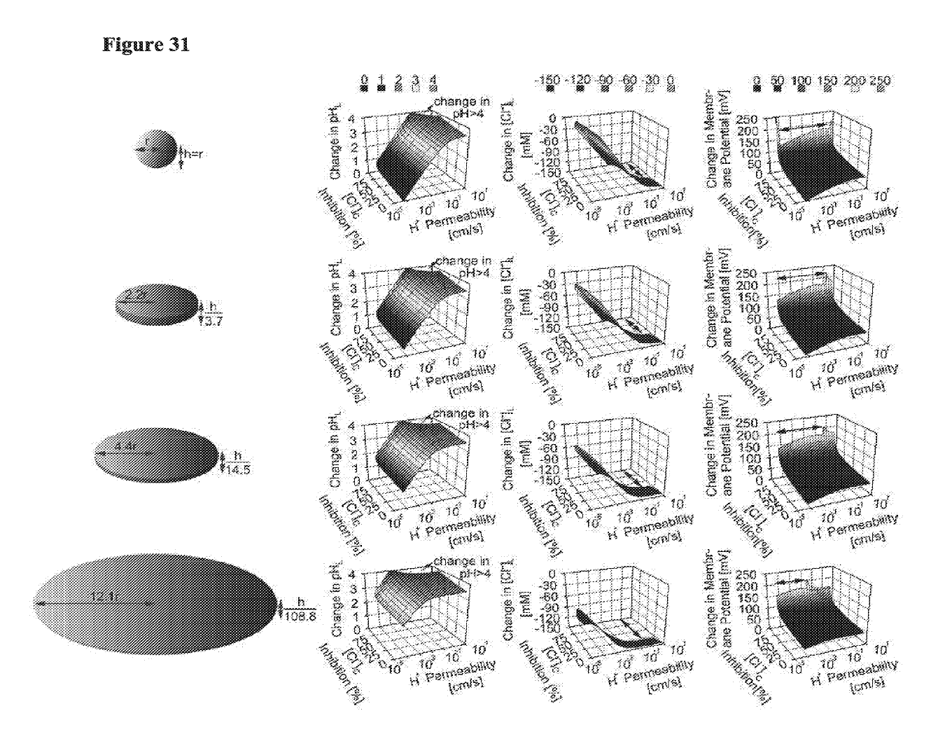

Although such increment in membrane potential is associated with the increment in lysosomal pH and reduction in lysosomal chloride accumulation, greater perturbations in both variables (>4 pH unit increment in lysosomal pH and >150 mM reduction in lysosomal chloride accumulation) were observed when at least either the V-ATPase number (FIGS. 25B, 25C, 26B, 27 and 28) or membrane proton permeability (FIGS. 25C, 28, 30 and 31) were maximally altered from their respective baseline values.

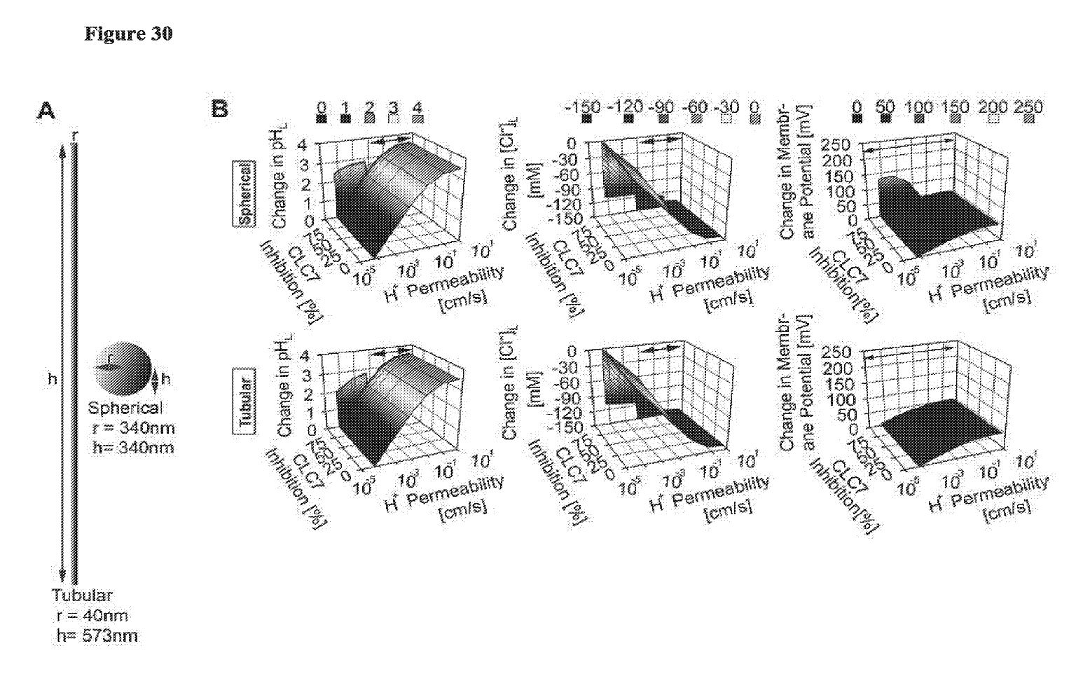

Following the simultaneous reduction of both V-ATPase and CLC7 numbers, up to 142.2 mM reduction of lysosomal chloride accumulation was observed to the extent of chloride efflux, in spherical as well as tubular lysosomes (FIG. 25B). Beyond the perturbation of lysosomal chloride level, up to 3.54 pH unit increment in lysosomal pH was also observed. Moreover, similar perturbation of lysosomal physiology was observed when the effect of the simultaneous alteration of membrane proton permeability and CLC7 number was simulated (FIG. 30). This indicates that the presence of lysosomal ion stressors, such as V-ATPase number and membrane proton permeability stressors which directly affect proton homeostasis, alone or in combination with lysosomal ion stressors, such as cytoplasmic Cl and CLC7 number stressors, which directly affect chloride homeostasis, perturb lysosomal physiology in a very similar manner.

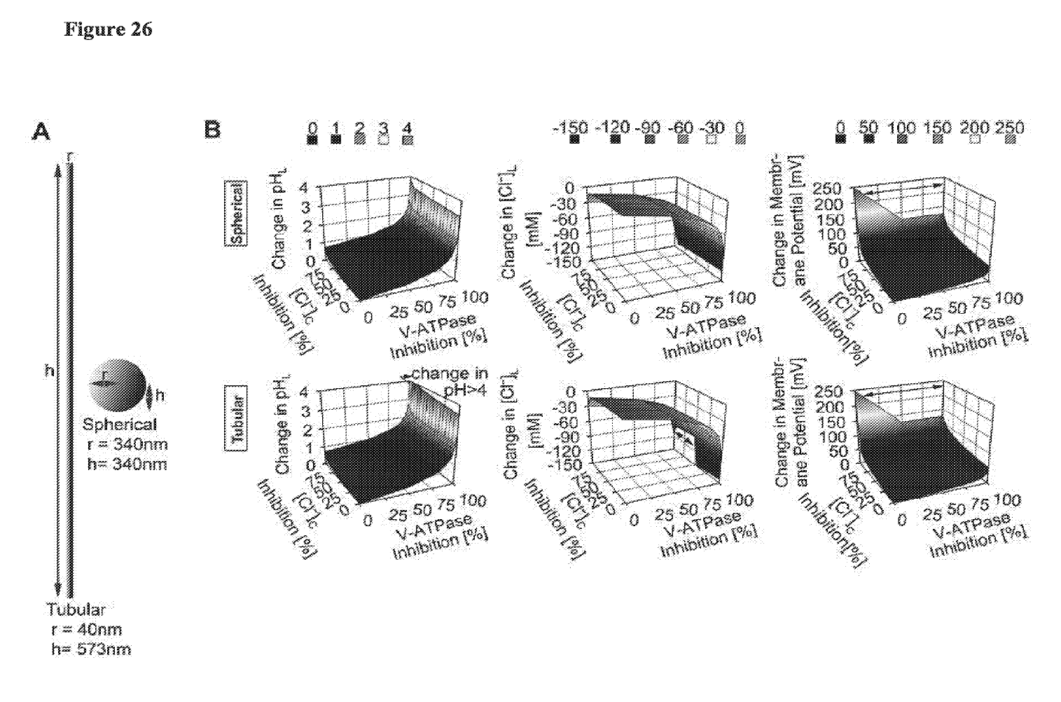

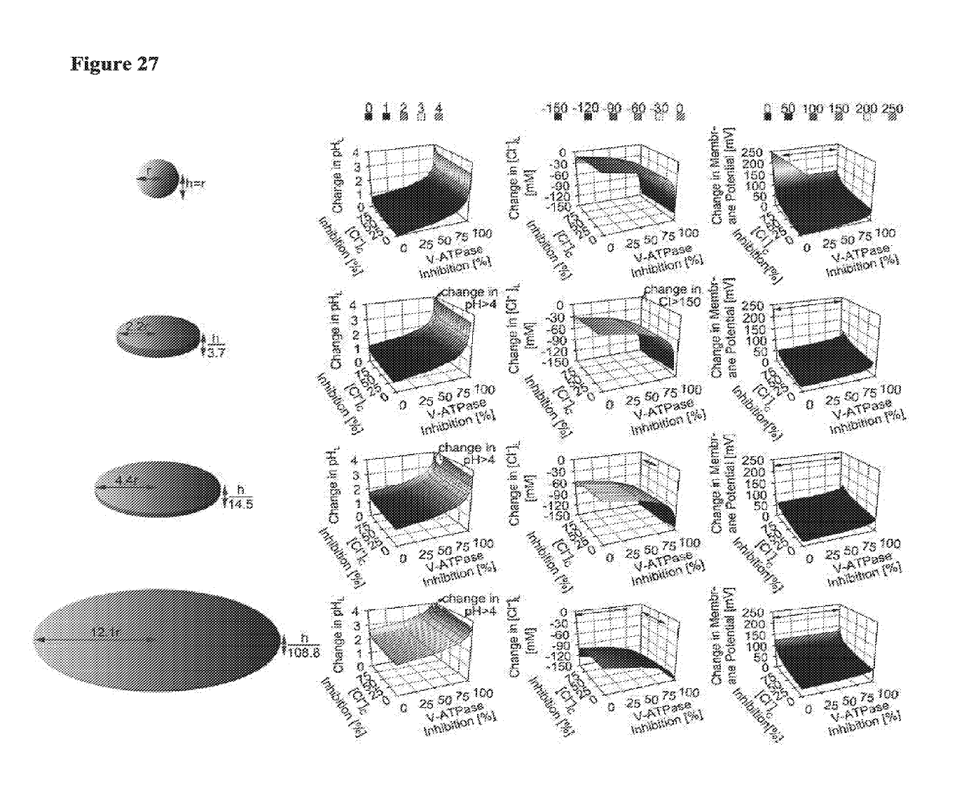

Regardless of lysosomal morphology, perturbations of lysosomal pH, Cl and membrane potential were generally greatest when either V-ATPase number or membrane proton permeability was simultaneously lowered or increased, respectively, along with the lowering of cytoplasmic chloride concentration from baseline values (FIGS. 26, 27, and 31). More specifically, the perturbations were greater in tubular (FIG. 26) and disc-shaped lysosomes with radial expansion >2 fold (FIG. 27) than in spherical lysosome. This phenomenon was characterized by >4 pH unit increment in lysosomal pH, >150 mM reduction in lysosomal chloride accumulation, and >250 mV increment in membrane potential (FIGS. 26 and 27). When comparing whether V-ATPase number, membrane proton permeability, or cytoplasmic chloride level affected the aforementioned perturbation the most, it is evident that either the lowering of V-ATPase number or the increment of proton permeability similarly exerted more significant effect on lysosomal physiology (including the reduction in lysosomal chloride accumulation) than that of the lowering of cytoplasmic chloride concentration. This further corroborates the finding regarding the most significant role of net lysosomal proton flux on the regulation of lysosomal physiology.

This example describes a mathematical modeling approach to explore the effect of various lysosomal ion and morphology stress inducers on lysosomal physiology. The complex dynamics of perturbation of lysosomal physiology in the presence of individual as well as combination of lysosomal stressors were characterized. The stressors reflect the cell-type dependent heterogeneous lysosomal size and shape distribution, as well as lysosomal membrane protein (such as V-ATPase and CLC7) expression levels and ion contents (Saftig P, et al., Nat Rev Mol Cell Biol. 2009;10(9):623-35). By altering any combination of these parameters, variation in the net proton flux with respect to lysosomal surface area was identified as the key parameter affecting lysosomal stress response.

All publications and patents mentioned in the present application are herein incorporated by reference. Various modification and variation of the described methods and compositions of the disclosure will be apparent to those skilled in the art without departing from the scope and spirit of the disclosure. Although the disclosure has been described in connection with specific preferred embodiments, it should be understood that the disclosure as claimed should not be unduly limited to such specific embodiments. Indeed, various modifications of the described modes for carrying out the disclosure that are obvious to those skilled in the relevant fields are intended to be within the scope of the following claims.

1. A composition comprising a compound comprising a physiologically insoluble hydrochloride salt of a weakly basic molecule.

2. The composition of claim 1, wherein said compound has a form selected from the group consisting of an aggregate, an inclusion, a particulate, and a crystal.

3. The composition of claim 1, wherein said compound is stable in a lysosome.

4. The composition of claim 1, wherein said compound has a pH max higher than the pH of a lysosome.

5. The composition of claim 1, wherein said composition is membrane-impermeant.

6. The composition of claim 1, wherein said compound further comprises an organic or inorganic coformer, counterion, solvent or excipient molecule.

7. The composition of claim 2, wherein said crystal is selected from the group consisting of a crystal with a 1:2 ratio of drug molecules to chloride ions and a crystal with a 1:1:2 ratio of drug molecules to chloride ions to MeOH.

8. The composition of claim 7, wherein the density of said crystals is between 1.35-1.5 g/ml.

9. The composition of claim 1, wherein said small pharmaceutical agent is clofazimine.

10. The composition of claim 1, wherein said composition further comprises a lipid.

11. The composition of claim 10, wherein said pharmaceutical agent is encapsulated by a liposome comprising said lipid.

12. The composition of claim 10, wherein said lipid is selected from the group consisting of phosphatidylcholine, cholesterol, phosphatidylethanolamine, phosphatidylglycerol, phosphatidylinositol, phosphatidylserine, sphingomyelin, cardiolipin, dioleoylphosphatidylglycerol (DOPG), diacylphosphatidylcholine, diacylphosphatidylethanolamine, ceramide, sphingomyelin, cephalin, cholesterol, cerebrosides, diacylglycerols, dioleoylphosphatidylcholine (DOPC), dimyristoylphosphatidylcholine (DMPC), and dioleoylphosphatidylserine (DOPS), diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palmitoyloleoyl-phosphatidylethanolamine (POPE) palmitoyloleoylphosphatidylcholine (POPC), egg phosphatidylcholine (EPC), di stearoylphosphatidylcholine (DSPC), dipalmitoylphosphatidylcholine (DPPC), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, dioctadecyldimethyl ammonium bromide, polyethylene glycol (PEG), and PEG modified lipids.

13. The composition of claim 1, wherein said composition further comprises one or more of a non-ionic surfactant, a niosome, a polymer, a protein, and a carbohydrate.

14. The composition of claim 10, wherein said lipid is modified to comprise a targeting agent selected from the group consisting of antibodies, mannose, folate, and transferrin.

15-21. (canceled)

22. A method of treating a disease in a subject, comprising:
administering the composition of claim 1 to a subject diagnosed with a disease.

23. The method of claim 22, wherein said administering reduces or eliminates symptoms of said disease.

24. The method of claim 22, wherein said disease is an inflammatory disease.

25. The method of claim 24, wherein said disease is acute or chronic.

26. The method of claim 22, wherein said disease is selected from the group consisting of asthma, bronchiolitis, bronchiolitis obliterans, chronic obstructive pulmonary disease (COPD), bronchitis, emphysema, hypersensitivity pneumonitis, idiopathic pulmonary fibrosis, pneumoconiosis, silicosis, meningitis, sepsis, malaria, rheumatoid osteoarthritis, psoriasis, acute respiratory disease syndrome, inflammatory bowel disease, multiple sclerosis, joint inflammation, reactive arthritis, hay fever, atherosclerosis, rheumatoid arthritis, bursitis, gouty arthritis, osteoarthritis, polymyalgia rheumatic arthritis, septic arthritis, infectious arthritis, asthma, autoimmune diseases, chronic inflammation, chronic prostatitis, glomerulonephritis, nephritis, inflammatory bowel diseases, pelvic inflammatory disease, reperfusion injury, transplant rejection, vasculitis, myocarditis, colitis, appendicitis, peptic ulcer, gastric ulcer, duodenal ulcer, peritonitis, pancreatitis, ulcerative colitis, seudomembranous colitis, acute colitis, ischemic colitis, diverticulitis, epiglottitis, achalasia, cholangitis, cholecystitits, hepatitis, Crohn's disease, enteritis, Whipple's disease, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, pneumonitits, pneumoultramicroscopic silicovolcanoconiosis, alvealitis, bronchiolitis, pharyngitis, pleurisy, sinusitis, influenza, respiratory syncytial virus infection, HIV infection, hepatitis B virus infection, hepatitis C virus infection, herpes virus infection disseminated bacteremia, Dengue fever, candidiasis, malaria, filariasis, amebiasis, hydatidcysts, burns, dermatitis, dermatomyositis, sunburn, urticaria, Warts, Wheals, vasulitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, adult respiratory distress syndrome, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thyroiditis, systemic lupus erythematosis, Goodpasture's syndrome, Behcet's syndrome, allograft rejection, graft-versus-host disease, Type I diabetes, Type II diabetes, ankylosing spondylitis, Berger's disease, Reiter's syndrome, Hodgkin's disease, ileus, hypertension, irritable bowel syndrome, myocardial infarction, sleeplessness, anxiety, local inflammation, and stent thrombosis.

27. The method of claim 26, wherein said disease is caused by infection by a microorganism selected from the group consisting of Staphylococcus aureus, Streptococcus, Streptococcus pneumonia, Neisseria gonorrhoeae, Mycobacterium tuberculosis, Borrelia burgdorferi, and Haemophilus influenza.

28-31. (canceled)
